(19) United States
(12) Patent Application Publication
(10) Pub. No.: US 2025/0123268 A1
(43) Pub. Date: Apr. 17, 2025

(54) COMPOSITIONS AND METHODS FOR ACCELERATING DEVELOPMENT OF CANCER THERAPEUTICS

(71) Applicant: NetNoids Rx Laboratories, LLC, Houston, TX (US)

(72) Inventors: Bhuvanesh Dave, Pearland, TX (US); Preethi Samuel, Pearland, TX (US)

(73) Assignee: NetNoids Rx Laboratories, LLC, Houston, TX (US)

(21) Appl. No.: 18/917,986

(22) Filed: Oct. 16, 2024

Related U.S. Application Data
(60) Provisional application No. 63/544,566, filed on Oct. 17, 2023.

(51) Int. Cl. G01N 33/50

(52) U.S. Cl. CPC G01N 33/5011

(57) ABSTRACT

Compositions and methods are provided for in vitro culture of human solid tumors as 3-dimensional patient derived tumoroids (PDT) co-cultured with immune cells to recapitulate the cellular architecture of the tumor sample from which they were derived. The construct of cells is supplied with oxygen from a media including oxygen. Disclosed are methods of using the PDTs disclosed herein to discover and validate cancer therapeutics.

### CROSS REFERENCE TO RELATED APPLICATIONS

This application claims the benefit of U.S. Provisional Patent Application No. 63/544,566 filed on Oct. 17, 2023 and entitled “CONSTRUCT OF CELLS AND RELATED SYSTEMS AND METHODS,” the entire contents of which are incorporated by reference herein.

### TECHNICAL FIELD

The disclosure relates to tumoroid-immune cell co-cultures and their use in the investigation of treatment for diseases.

### BACKGROUND

Despite recent progress, the development of targeted cancer therapies is rate limited by a lack of relevant data on drug effectiveness and 95% of known cancers have no targeted treatment. Non-targeted treatment frequently results in increased recurrence, decreased quality of life, and increased mortality. For example, existing options include (1) mouse models (human models are needed); (2) 2-dimensional cell culture; and (3) basic organoids (tumor cells do not grow in isolation). This results in high failure rates (96.7%) for drugs at the regulatory approval stage which in turn results in loss of millions of dollars spent across multiple years.

Unfortunately, there are no robust in vitro models for the study of tumors that recapitulates the complex in vivo physical architecture of a tumor. Thus, there is a need for improved methods for preparing organoid/tumoroid-immune cell co-cultures and methods for using these co-cultures in drug screening, particularly a system in which the interaction between disease cells and immune cells is leveraged to investigate an increased array of drugs with high-throughput capability.

### SUMMARY

Discloses are methods, compositions, systems, and kits that solves some of these unmet needs by providing highly translatable pre-clinical human efficacy data that is 85% vs. 30% translatable and in a shorter timeframe (5 months vs. 24 months) than other current methods.

Disclosed is a research tool for drug discovery. Disclosed are Patient Derived Tumoroids (PDTs). Disclosed are Patient Derived Tumoroids (PDTs) for use in for drug discovery and/or developing treatments for cancer. Disclosed are COMPASS (Custom Organoid Modelling Platform for Accurate & Speedy Solutions) platforms. Disclosed are tumoroids/PDTs-immune cell co-cultures comprising, consisting of, or consisting essentially a of tumor immune microenvironment (TME). Disclosed are tumoroids/PDTs-immune cell co-cultures in media comprising, consisting of, or consisting essentially of oxygen. Disclosed are tumoroids/PDTs-immune cell co-cultures in media comprising, consisting of, or consisting essentially of oxygen and PBMC. Disclosed are tumoroids/PDTs-immune cell co-cultures with TME and functional immunocompetence. Disclosed are tumoroids/PDTs-immune cell co-cultures with TME in a media comprising, consisting of, or consisting essentially of physiological oxygenation. Disclosed are tumoroids/PDTs-immune cell co-cultures with TME, functional immunocompetence, in a media comprising, consisting of, or consisting essentially of oxygen. Disclosed are tumoroids/PDTs-immune cell co-cultures comprising, consisting of, or consisting essentially of a tumor immune microenvironment (TME) in media comprising, consisting of, or consisting essentially of oxygen and PBMC.

### Construct of Cells

Disclosed is a construct of cells. Disclosed is a PDT. In some embodiments, the PDTs are applicable for use with any cancer indication.

In some embodiments, the construct of cells is derived from a living object and comprise: a plurality of cells derived from a living object; at least one immune cell; and a culturing matrix comprising, consisting of, or consisting essentially of exogenous laminin. In some embodiments, the construct of cells comprises immune cells. In some embodiments, the at least one immune cell is derived from the living object. In some embodiments, the at least one immune cell is not derived from the living object.

In some embodiments, the construct of cells derived from a living object, comprises: a plurality of cells derived from a living object; and a culturing matrix comprising, consisting of, or consisting essentially of exogenous laminin wherein the construct of cells is supplied with oxygen from a media including oxygen. In some embodiments, the construct of cells comprises S-PDT-V2, S-PDT-V3b and/or S-PDT-V3d cells and a media. In some embodiments, the construct of cells comprises S-PDT-V2, S-PDT-V3b and/or S-PDT-V3d cells and blood substitute. In some embodiments, the at least one immune cell is derived from the living object. In some embodiments, the at least one immune cell is not derived from the living object.

In some embodiments, the construct of cells derived from a living object, comprises: a plurality of cells derived from a living object; at least one immune cell; and a culturing matrix comprising, consisting of, or consisting essentially of exogenous laminin, wherein the construct of cells is supplied with oxygen from a media including oxygen. In some embodiments, the at least one immune cell is derived from the living object. In some embodiments, the at least one immune cell is not derived from the living object.

In some embodiments, the PDT comprises a TME. In some embodiments, the Tumor MicroEnvironment (TME) enables recapitulation of patient-specific intra-tumoral interactions. This in turn enables a fast-track search for novel cancer treatments.

### Amounts of Immune Cells

In some embodiments, the at least one immune cell constitutes about 1% to about 90% of the construct of cells. In some embodiments, the at least one immune cell constitutes about 3% to about 80% of the construct of cells. In some embodiments, the at least one immune cell constitutes about 5% to about 70% of the construct of cells. In some embodiments, the at least one immune cell constitutes about 8% to about 65% of the construct of cells. In some embodiments, the at least one immune cell constitutes about 10% to about 60% of the construct of cells. In some embodiments, the at least one immune cell constitutes about 12% to about 55% of the construct of cells. In some embodiments, the at least one immune cell constitutes about 15% to about 50% of the construct of cells. In some embodiments, the PDT has functional immunocompetence. In some embodiments, the PDT adds the entire immune system. In some embodiments, the PDT comprises, or consists essentially of peripheral blood mononuclear cells. In some embodiments, these peripheral blood mononuclear cells are added at the time of development and growth of the tumoroid/PDT in order to incorporate them into the tumor microenvironment and allow them to be part of the tumoroid/PDT instead of being added for co-culture. In some embodiments, these peripheral blood mononuclear cells are added during co-culture. These immune competent tumoroids/PDT determines the impact of the functionally relevant immune system on therapeutics, and the effect of immune cross-talk on immune modulating drugs as well as standard small molecules and large molecules.

In some embodiments, the PDT system comprises physiological oxygenation. Historically oxygenation in cell culture is directly through diffusion from air in a cell culture incubator. In the human body the oxygen comes in the form of dissolved oxygen in blood by binding to hemoglobin and being releases in tissues by signals from them. This process is replicated in the PDT physiological oxygenation framework where synthetic hemoglobin is added at the base of the PDT in a very viscous liquid which does not mix with media but can circulate oxygen to the tumoroids/PDT in the same manner as the human system using the physiological pump. The tumoroids/PDT can be tested in presence and absence of oxygenation and/or functional immune system. This allows for a first instance of preclinical testing where the human-on-human components can be varied to define different patient situations and test therapeutic options. In some embodiments, drug treatment on the same patient derived tumoroid system under different TME conditions like presence or absence of physiological oxygenation and/or functional immune system produces different results.

### Types of Immune Cells

In some embodiments, the at least one immune cell includes a mononuclear cell. In some embodiments, the at least one immune cell includes a lymphocyte and/or a monocyte. In some embodiments, the at least one immune cell includes a peripheral blood mononuclear cell (PBMC). In some embodiments, the at least one immune cell includes T Cells (CD3+), B cells (CD20), Macrophages (CD68), NK cells (CD16), or a combination thereof. In some embodiments, the at least one immune cell includes a mononuclear cell, lymphocyte, monocyte, PBMC, T Cells (CD3+), B cells (CD20), Macrophages (CD68), NK cells (CD16), or a combination thereof.

### Functional Aspect of the Immune Cell Construct

In some embodiments, the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof. In some embodiments, the information contains spatial information, temporal information, or a combination thereof. In some embodiments, the information is based on at least two different indications from two different immune cells among the at least one immune cells. In some embodiments, the at least one immune cell is tagged with a tagging molecule to indicate the information. In some embodiments, the tagging molecule includes a fluorescent molecule, radioactive isotope molecule, enzyme labels, coloring molecule, immunostaining molecule, biotin-avidin complexes, chemiluminescence, or a combination thereof.

### Static Oxygen Exchange

In some embodiments, the oxygen is supplied externally to the media via a gas-liquid interface. Specifically, air is added to the cells with 5% CO2 for growth. Addition of oxygen using a synthetic hemoglobin allows put tumors in context of physiologically relevant/equivalent oxygen concentrations which cannot are not present in historical cell culture. The growth rate of the tumoroids is slowed down and closer to real tumors in presence of physiologically relevant oxygen making the disclosed PDTs a more relevant tumor model systems. In some embodiments, the system is closed. In some embodiments, the PDT has a slower growth rate than a tumor in cell culture. In some embodiments, the PDT has a faster growth rate than a tumor in cell culture. In some embodiments, gas forming the gas-liquid interface has a lower oxygen concentration than ambient air. In some embodiments, gas forming the gas-liquid interface has a higher oxygen concentration than ambient air. In some embodiments, gas forming the gas-liquid interface includes from about 0% to about 90% of oxygen. In some embodiments, gas forming the gas-liquid interface includes from about 0% to about 80% of oxygen. In some embodiments, gas forming the gas-liquid interface includes from about 0% to about 70% of oxygen. In some embodiments, gas forming the gas-liquid interface includes from about 0% to about 60% of oxygen. In some embodiments, gas forming the gas-liquid interface includes from about 0% to about 50% of oxygen. In some embodiments, gas forming the gas-liquid interface includes from about 0% to about 40% of oxygen. In some embodiments, gas forming the gas-liquid interface includes from about 0% to about 35% of oxygen. In some embodiments, gas forming the gas-liquid interface includes from about 0% to about 30% of oxygen. In some embodiments, gas forming the gas-liquid interface includes from about 0% to about 25% of oxygen. In some embodiments, gas forming the gas-liquid interface includes from about 0% to about 22% of oxygen. In some embodiments, gas forming the gas-liquid interface includes from about 0% to about 20% of oxygen. In some embodiments, the liquid-liquid interface is above the tumoroid, below the tumoroid or bisects the tumoroid. In some embodiments, the liquid-liquid interface is formed by interfacing the growth media and synthetic hemoglobin.

### Blood Substitute (Artificial Blood)

In some embodiments, at least a portion of the media is replaced. In some embodiments, at least a portion of the media is infused into a system containing the construct of cells for a first predetermined period of time. In some embodiments, at least a portion of the media is withdrawn from the system for a second predetermined period of time. In some embodiments, the media includes a blood substitute. In some embodiments, the media includes hemoglobin. In some embodiments, wherein the media includes synthetic hemoglobin. In some embodiments, the media comprises a first media and a second media different from the first media. In some embodiments, the second media includes blood substitute. In some embodiments, the second media includes hemoglobin. In some embodiments, the second media includes artificial hemoglobin. In some embodiments, the density of the first media is lower than the second media. In some embodiments, the first media is in contact with cells of the plurality of cells. In some embodiments, the second media forms a liquid-liquid interface with the first media. In some embodiments, the concentration of oxygen in the second media is from about 0 μM to about 300 μM. In some embodiments, the concentration of oxygen in the second media is from about 0 μM to about 270 μM. In some embodiments, the concentration of oxygen in the second media is from about 0 M to about 250 μM. In some embodiments, the concentration of oxygen in the second media is from about 0 μM to about 240 M. In some embodiments, the concentration of oxygen in the second media is from about 0 μM to about 235 μM. In some embodiments, the concentration of oxygen in the second media is from about 0 μM to about 230 μM. In some embodiments, the concentration of oxygen in the second media is from about 0 μM to about 225 μM. In some embodiments, the concentration of oxygen in the second media is from about 0 μM to about 200 μM. In some embodiments, the concentration of oxygen in the second media is from about 0 μM to about 175 μM. In some embodiments, the concentration of oxygen in the second media is from about 0 μM to about 150 μM. In some embodiments, the concentration of oxygen in the second media is from about 0 M to about 125 μM. In some embodiments, the concentration of oxygen in the second media is from about 0 μM to about 100 μM. In some embodiments, the concentration of oxygen in the second media is from about 0 μM to about 75 μM. In some embodiments, the concentration of oxygen in the second media is from about 0 μM to about 50 μM. In some embodiments, the concentration of oxygen in the second media is from about 0 μM to about 25 μM. In some embodiments, the concentration of oxygen in the second media is from about 25 μM to about 300 μM. In some embodiments, the concentration of oxygen in the second media is from about 25 μM to about 270 μM. In some embodiments, the concentration of oxygen in the second media is from about 25 μM to about 250 μM. In some embodiments, the concentration of oxygen in the second media is from about 25 μM to about 240 μM. In some embodiments, the concentration of oxygen in the second media is from about 25 μM to about 235 μM. In some embodiments, the concentration of oxygen in the second media is from about 25 μM to about 230 μM. In some embodiments, the concentration of oxygen in the second media is from about 25 μM to about 225 μM. In some embodiments, the concentration of oxygen in the second media is from about 25 μM to about 200 μM. In some embodiments, the concentration of oxygen in the second media is from about 25 μM to about 175 μM. In some embodiments, the concentration of oxygen in the second media is from about 25 μM to about 150 μM. In some embodiments, the concentration of oxygen in the second media is from about 25 μM to about 125 μM. In some embodiments, the concentration of oxygen in the second media is from about 25 μM to about 100 μM. In some embodiments, the concentration of oxygen in the second media is from about 25 μM to about 75 μM. In some embodiments, the concentration of oxygen in the second media is from about 25 μM to about 50 M.

In some embodiments, the first media covers the PDT, and the second media does not cover the PDT. In some embodiments, the first media does not cover the PDT and the second media covers the PDT.

### Components of Matrigel™

In some embodiments, the culturing matrix comprises exogenous entactin. In some embodiments, the culturing matrix comprises Matrigel™. In some embodiments, the culturing matrix induces more cell proliferation of the construct of cells within a given period of time. In some embodiments, the culturing matrix induces more mass of the construct of cells within a given period of time. In some embodiments, the culturing matrix induces more volume of the construct of cells within a given period of time. In some embodiments, the construct of cells includes a three-dimensional (3D) structure. In some embodiments, the construct of cells includes a three-dimensional (3D) patient-derived tumoroid (PDTs). In some embodiments, the plurality of cells is derived from a tumor of the living object. In some embodiments, the plurality of cells includes cancer cells. In some embodiments, the plurality of cells includes sarcoma cells, or gallbladder cancer. In some embodiments, the plurality of cells includes Human Uterine Adenosarcoma cells.

### A System for a Construct of Cells

The emergence of cancer organoid technology with the intrinsic advantage of retaining the heterogeneity of original tumors has provided a unique opportunity to improve basic and clinical cancer research. Generating these organoids is cost-effective, straightforward, and is completed within a month. Moreover, they are cultured in microplates suitable for high throughput assays. Yet, there's a notable absence of organoid models that accurately capture the interaction between tumors and immune cells in the tumor microenvironment (TME). Existing models often fall short in replicating the intricate diversity and structure of the TME, especially in enabling the tumoroid-immune cell co-culture of primary tumor epithelium with native infiltrating immune cells without artificial reconstruction.

Disclosed is Custom Organoid Modeling Platform for Accurate and Speedy Solutions (COMPASS). This versatile toolkit of organoid models ensures robust prediction of clinical outcomes through precision preclinical research. By employing human-relevant ex-vivo models, COMPASS is able to accelerate drug discovery with an efficacy-driven approach.

Disclosed are modality-agnostic bio-banked COMPASS models concerning functional immunity and physiological oxygenation. Disclosed are modality-agnostic bio-banked COMPASS models concerning functional immunity and physiological oxygenation in the context of cancer. In some embodiments, the cancer is ovarian, neuroendocrine, gall bladder, breast, colorectal, glioblastoma, gastric, esophageal, sarcoma, bladder, prostate, colorectal, ovarian, pancreatic and/or lung cancer.

In some embodiments, the COMPASS platform is modality agnostic meaning it is used to generate data on any therapeutic modality desirable of testing. In some embodiments, the platform enables testing of immune-based therapeutics. In some embodiments, the platform enables testing of pH indexed therapeutic response data with or without the immune system. Currently, there is a lack of model systems that capture the biology of human tumors. The COMPASS platform is a model system that recapitulates and mimics human tumors. Further there is a lack of models that provide human efficacy data at the preclinical stage. The COMPASS platform provides highly translatable human efficacy data.

In some embodiments, the COMPASS system retains TME close to the original patient tumor. In some embodiments, this system avoids enzymatic separation, or disintegration into single cells and exclusion of stromal components of the tumor to enable the mechanically separated tumor chunks to successfully reconstitute themselves to its original structural and functional form.

In some embodiments, the COMPASS system has qualitatively and/or quantitatively measurable functional immuno-competence. In some embodiments, the built-in functional immuno-competence provides tumor heterogeneity that enables investigation crucial for understanding resistance mechanisms and improving treatment effectiveness to immunotherapies in patients. As such, the COMPASS system is a comprehensive tool to study developmental and functional phases of engineered cell therapies, immune component infiltration and mechanisms of immunomodulation.

In some embodiments, the COMPASS system has physiological oxygenation with or without functional immuno-competence. In some embodiments, the COMPASS system is scaffold-based. In some embodiments, the COMPASS system is not scaffold-based.

In some embodiments, the COMPASS system fast tracks a partners' discovery and validation journey for therapeutic options for cancers. A main objective of the COMPASS system is to enable faster development of cancer therapeutics using a human-on-human cancer model. The end goal is the development of targeted drug therapies for cancers.

Disclosed is a system for a construct of cells, comprising, consisting of, or consisting essentially of a tissue container to grow a construct of cells constructed in the tissue container, wherein the construct comprises: a plurality of cells derived from a living object; at least one immune cell; and a culturing matrix comprising, consisting of, or consisting essentially of exogenous laminin. Disclosed is a system to contain a construct of cells, comprising, consisting of, or consisting essentially of a tissue container to grow a construct of cells constructed in the tissue container, wherein the construct comprises: a plurality of cells derived from a living object; and a culturing matrix comprising, consisting of, or consisting essentially of exogenous laminin, wherein the construct of cells is supplied with oxygen from a media including oxygen. Disclosed is a system for a construct of cells, comprising, consisting of, or consisting essentially of: a tissue container to grow a construct of cells constructed in the tissue container, wherein the construct comprises: a plurality of cells derived from a living object; at least one immune cell; and a culturing matrix comprising, consisting of, or consisting essentially of exogenous laminin, wherein the construct of cells is to be supplied with oxygen from a media including oxygen.

In some embodiments, the at least one immune cell constitutes about 1% to about 90% of the construct of cells. In some embodiments, the at least one immune cell constitutes about 3% to about 80% of the construct of cells. In some embodiments, the at least one immune cell constitutes about 5% to about 70% of the construct of cells. In some embodiments, the at least one immune cell constitutes about 8% to about 65% of the construct of cells. In some embodiments, the at least one immune cell constitutes about 10% to about 60% of the construct of cells. In some embodiments, the at least one immune cell constitutes about 12% to about 55% of the construct of cells. In some embodiments, the at least one immune cell constitutes about 15% to about 50% of the construct of cells. In some embodiments, the at least one immune cell includes a mononuclear cell. In some embodiments, the at least one immune cell includes a lymphocyte or a monocyte. In some embodiments, the at least one immune cell includes a peripheral blood mononuclear cell (PBMC). In some embodiments, the at least one immune cell includes T Cells (CD3+), B cells (CD20), Macrophages (CD68), NK cells (CD16), or a combination thereof. In some embodiments, the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof. In some embodiments, the information contains spatial information, temporal information, or a combination thereof. In some embodiments, the information is based on at least two different indications from two different immune cells among the at least one immune cells. In some embodiments, the at least one immune cell is tagged with a tagging molecule to indicate the information.

In some embodiments, the tagging molecule includes a fluorescent molecule, radioactive isotope molecule, enzyme labels, coloring molecule, immunostaining molecule, biotin-avidin complexes, chemiluminescence, or a combination thereof. In some embodiments, the oxygen is perfused externally to the media via a gas-liquid interface. In some embodiments, gas forming the gas-liquid interface has lower oxygen concentration than ambient air. In some embodiments, gas forming the gas-liquid interface has higher oxygen concentration than ambient air. In some embodiments, gas forming the gas-liquid interface includes from about 0% to about 90% of oxygen. In some embodiments, gas forming the gas-liquid interface includes from about 0% to about 80% of oxygen. In some embodiments, gas forming the gas-liquid interface includes from about 0% to about 70% of oxygen. In some embodiments, gas forming the gas-liquid interface includes from about 0% to about 60% of oxygen. In some embodiments, gas forming the gas-liquid interface includes from about 0% to about 50% of oxygen. In some embodiments, gas forming the gas-liquid interface includes from about 0% to about 40% of oxygen. In some embodiments, gas forming the gas-liquid interface includes from about 0% to about 35% of oxygen. In some embodiments, gas forming the gas-liquid interface includes from about 0% to about 30% of oxygen. In some embodiments, gas forming the gas-liquid interface includes from about 0% to about 25% of oxygen. In some embodiments, gas forming the gas-liquid interface includes from about 0% to about 22% of oxygen. In some embodiments, gas forming the gas-liquid interface includes from about 0% to about 20% of oxygen. In some embodiments, at least a portion of the media in the system is replaced. In some embodiments, at least a portion of the media is infused into a system containing the construct of cells for a first predetermined period of time. In some embodiments, at least a portion of the media is withdrawn from the system for a second predetermined period of time. In some embodiments, the media includes a blood substitute. In some embodiments, the media includes hemoglobin. In some embodiments, the media includes synthetic hemoglobin. In some embodiments, the media comprises a first media and a second media different from the first media. In some embodiments, second media includes blood substitute. In some embodiments, second media includes hemoglobin. In some embodiments, second media includes artificial hemoglobin. In some embodiments, the density of the first media is lower than the second media. In some embodiments, the first media is in contact with cells of the plurality of cells. In some embodiments, the second media forms a liquid-liquid interface with the first media. In some embodiments, the concentration of oxygen in the second media is from about 0 μM to about 300 μM. In some embodiments, the concentration of oxygen in the second media is from about 0 μM to about 270 μM. In some embodiments, the concentration of oxygen in the second media is from about 0 μM to about 250 μM. In some embodiments, the concentration of oxygen in the second media is from about 0 μM to about 240 μM. In some embodiments, concentration of oxygen in the second media is from about 0 μM to about 235 μM. In some embodiments, the concentration of oxygen in the second media is from about 0 μM to about 230 μM. In some embodiments, the concentration of oxygen in the second media is from about 0 μM to about 225 μM. In some embodiments, the concentration of oxygen in the second media is from about 0 μM to about 200 μM. In some embodiments, the concentration of oxygen in the second media is from about 0 μM to about 175 μM. In some embodiments, the concentration of oxygen in the second media is from about 0 μM to about 150 μM. In some embodiments, the concentration of oxygen in the second media is from about 0 μM to about 125 μM. In some embodiments, the concentration of oxygen in the second media is from about 0 μM to about 100 μM. In some embodiments, the concentration of oxygen in the second media is from about 0 μM to about 75 μM. In some embodiments, the concentration of oxygen in the second media is from about 0 M to about 50 μM. In some embodiments, the concentration of oxygen in the second media is from about 0 μM to about 25 μM. In some embodiments, the concentration of oxygen in the second media is from about 25 μM to about 300 μM. In some embodiments, the concentration of oxygen in the second media is from about 25 μM to about 270 μM. In some embodiments, the concentration of oxygen in the second media is from about 25 μM to about 250 μM. In some embodiments, the concentration of oxygen in the second media is from about 25 μM to about 240 μM. In some embodiments, the concentration of oxygen in the second media is from about 25 μM to about 235 μM. In some embodiments, the concentration of oxygen in the second media is from about 25 μM to about 230 μM. In some embodiments, the concentration of oxygen in the second media is from about 25 μM to about 225 μM. In some embodiments, the concentration of oxygen in the second media is from about 25 μM to about 200 μM. In some embodiments, the concentration of oxygen in the second media is from about 25 μM to about 175 μM. In some embodiments, the concentration of oxygen in the second media is from about 25 μM to about 150 μM. In some embodiments, the concentration of oxygen in the second media is from about 25 μM to about 125 μM. In some embodiments, the concentration of oxygen in the second media is from about 25 μM to about 100 μM. In some embodiments, the concentration of oxygen in the second media is from about 25 μM to about 75 μM. In some embodiments, the concentration of oxygen in the second media is from about 25 μM to about 50 μM.

In some embodiments, the culturing matrix further comprises exogenous entactin. In some embodiments, the culturing matrix comprises Matrigel™. In some embodiments, the culturing matrix is to induce more cell proliferation of the construct of cells within a given period of time. In some embodiments, the culturing matrix is to induce more mass of the construct of cells within a given period of time. In some embodiments, the culturing matrix is to induce more volume of the construct of cells within a given period of time. In some embodiments, the construct of cells includes a three-dimensional (3D) structure.

In some embodiments, the construct of cells includes a three-dimensional (3D) patient-derived tumoroid (PDTs). In some embodiments, the plurality of cells is derived from a tumor of the living object. In some embodiments, the plurality of cells includes cancer cells. In some embodiments, the plurality of cells includes sarcoma cells, gallbladder cancer. In some embodiments, the plurality of cells includes Human Uterine Adenosarcoma cells.

In some embodiments, the construct of cells further comprises an inlet to allow a medium carrying oxygen to enter the system to perfuse oxygen to be supplied to the construct of cells. In some embodiments, construct of cells further comprises a well to contain the construct of the cells.

### Methods of Using a Construct of Cells

Disclosed is a method of evaluating a response of a plurality of cells derived from a living object against an external stimulus, the method comprising, consisting of, or consisting essentially of: culturing a construct of cells comprising, consisting of, or consisting essentially of a plurality of cells derived from a living object and at least one immune cell, in a culturing matrix comprising, consisting of, or consisting essentially of exogenous laminin; and subjecting the plurality of cultured cells to an external stimulus.

Disclosed is a method of evaluating a response of a plurality of cells derived from a living object against an external stimulus, the method comprising, consisting of, or consisting essentially of: culturing a construct of cells comprising, consisting of, or consisting essentially of a plurality of cells derived from a living object in a culturing matrix comprising, consisting of, or consisting essentially of exogenous laminin; and subjecting the plurality of cultured cells to an external stimulus. In some embodiments, the plurality of cells is supplied with oxygen from a media including oxygen.

Disclosed is a method of evaluating a response of a plurality of cells derived from a living object against an external stimulus, the method comprising, consisting of, or consisting essentially of: culturing a construct of cells comprising, consisting of, or consisting essentially of a plurality of cells derived from a living object and at least one immune cell, in a culturing matrix comprising, consisting of, or consisting essentially of exogenous laminin; and subjecting the plurality of cultured cells to an external stimulus, wherein the plurality of cells is supplied with oxygen from a media including oxygen.

In some embodiments, the at least one immune cell constitutes about 1% to about 90% of the construct of cells. In some embodiments, the at least one immune cell constitutes about 3% to about 80% of the construct of cells. In some embodiments, the at least one immune cell constitutes about 5% to about 70% of the construct of cells. In some embodiments, the at least one immune cell constitutes about 8% to about 65% of the construct of cells. In some embodiments, the at least one immune cell constitutes about 10% to about 60% of the construct of cells. In some embodiments, the at least one immune cell constitutes about 12% to about 55% of the construct of cells. In some embodiments, the at least one immune cell constitutes about 15% to about 50% of the construct of cells. In some embodiments, the at least one immune cell includes a mononuclear cell. In some embodiments, the at least one immune cell includes a lymphocyte or a monocyte. In some embodiments, the at least one immune cell includes a peripheral blood mononuclear cell (PBMC). In some embodiments, the at least one immune cell includes T Cells (CD3+), B cells (CD20), Macrophages (CD68), NK cells (CD16), or a combination thereof. In some embodiments, the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof.

In some embodiments, the information contains spatial information, temporal information, or a combination thereof. In some embodiments, the information is based on at least two different indications from two different immune cells among the at least one immune cells.

In some embodiments, the at least one immune cell is tagged with a tagging molecule to indicate the information. In some embodiments, the tagging molecule includes a fluorescent molecule, radioactive isotope molecule, enzyme labels, coloring molecule, immunostaining molecule, biotin-avidin complexes, chemiluminescence, or a combination thereof. In some embodiments, the oxygen is supplied externally to the media via a gas-liquid interface. In some embodiments, gas forming the gas-liquid interface has lower oxygen concentration than ambient air. In some embodiments, gas forming the gas-liquid interface has higher oxygen concentration than ambient air. In some embodiments, gas forming the gas-liquid interface includes from about 0% to about 90% of oxygen. In some embodiments, gas forming the gas-liquid interface includes from about 0% to about 80% of oxygen. In some embodiments, gas forming the gas-liquid interface includes from about 0% to about 70% of oxygen. In some embodiments, gas forming the gas-liquid interface includes from about 0% to about 60% of oxygen. In some embodiments, gas forming the gas-liquid interface includes from about 0% to about 50% of oxygen. In some embodiments, gas forming the gas-liquid interface includes from about 0% to about 40% of oxygen. In some embodiments, gas forming the gas-liquid interface includes from about 0% to about 35% of oxygen. In some embodiments, gas forming the gas-liquid interface includes from about 0% to about 30% of oxygen. In some embodiments, gas forming the gas-liquid interface includes from about 0% to about 25% of oxygen. In some embodiments, gas forming the gas-liquid interface includes from about 0% to about 22% of oxygen. In some embodiments, gas forming the gas-liquid interface includes from about 0% to about 20% of oxygen.

In some embodiments, at least a portion of the media is replaced. In some embodiments, at least a portion of the media is infused into a system containing the construct of cells for a first predetermined period of time. In some embodiments, at least a portion of the media is withdrawn from the system for a second predetermined period of time. In some embodiments, the media includes a blood substitute. In some embodiments, the media includes hemoglobin. In some embodiments, the media includes synthetic hemoglobin. In some embodiments, the media comprises a first media and a second media different from the first media. In some embodiments, the second media includes blood substitute. In some embodiments, the second media includes hemoglobin. In some embodiments, the second media includes artificial hemoglobin. In some embodiments, the density of the first media is lower than the second media. In some embodiments, the first media is in contact with cells of the plurality of cells. In some embodiments, the second media forms a liquid-liquid interface with the first media. In some embodiments, the concentration of oxygen in the second media is from about 0 μM to about 300 μM. In some embodiments, the concentration of oxygen in the second media is from about 0 μM to about 270 μM. In some embodiments, the concentration of oxygen in the second media is from about 0 M to about 250 μM. In some embodiments, the concentration of oxygen in the second media is from about 0 μM to about 240 M. In some embodiments, the concentration of oxygen in the second media is from about 0 μM to about 235 μM. In some embodiments, the concentration of oxygen in the second media is from about 0 μM to about 230 μM. In some embodiments, the concentration of oxygen in the second media is from about 0 μM to about 225 μM. In some embodiments, the concentration of oxygen in the second media is from about 0 μM to about 200 μM. In some embodiments, the concentration of oxygen in the second media is from about 0 μM to about 175 μM. In some embodiments, the concentration of oxygen in the second media is from about 0 μM to about 150 μM. In some embodiments, the concentration of oxygen in the second media is from about 0 μM to about 125 μM. In some embodiments, the concentration of oxygen in the second media is from about 0 μM to about 100 μM. In some embodiments, the concentration of oxygen in the second media is from about 0 μM to about 75 μM. In some embodiments, the concentration of oxygen in the second media is from about 0 μM to about 50 μM. In some embodiments, the concentration of oxygen in the second media is from about 0 μM to about 25 μM. In some embodiments, the concentration of oxygen in the second media is from about 25 μM to about 300 μM. In some embodiments, the concentration of oxygen in the second media is from about 25 μM to about 270 μM. In some embodiments, the concentration of oxygen in the second media is from about 25 μM to about 250 μM. In some embodiments, the concentration of oxygen in the second media is from about 25 μM to about 240 μM. In some embodiments, the concentration of oxygen in the second media is from about 25 μM to about 235 μM. In some embodiments, the concentration of oxygen in the second media is from about 25 μM to about 230 μM. In some embodiments, the concentration of oxygen in the second media is from about 25 μM to about 225 μM. In some embodiments, the concentration of oxygen in the second media is from about 25 μM to about 200 μM. In some embodiments, the concentration of oxygen in the second media is from about 25 μM to about 175 μM. In some embodiments, the concentration of oxygen in the second media is from about 25 μM to about 150 μM. In some embodiments, the concentration of oxygen in the second media is from about 25 μM to about 125 μM. In some embodiments, the concentration of oxygen in the second media is from about 25 μM to about 100 μM. In some embodiments, the concentration of oxygen in the second media is from about 25 μM to about 75 μM. In some embodiments, the concentration of oxygen in the second media is from about 25 μM to about 50 μM. In some embodiments, the external stimulus is based on a small molecule therapeutics. In some embodiments, the external stimulus is based on a large molecule therapeutics. In some embodiments, the culturing matrix further comprises exogenous entactin. In some embodiments, the culturing matrix comprises Matrigel™. In some embodiments, the culturing matrix induces more cell proliferation of the construct of cells within a given period of time. In some embodiments, the culturing matrix induces more mass of the construct of cells within a given period of time. In some embodiments, the culturing matrix induces more volume of the construct of cells within a given period of time. In some embodiments, the construct of cells includes a three-dimensional (3D) structure. In some embodiments, the construct of cells includes a three-dimensional (3D) patient-derived tumoroid (PDTs). In some embodiments, the plurality of cells is derived from a tumor of the living object. In some embodiments, the plurality of cells includes cancer cells. In some embodiments, the plurality of cells includes sarcoma cells, gallbladder cancer. In some embodiments, the plurality of cells includes Human Uterine Adenosarcoma cells.

### BRIEF DESCRIPTION OF DRAWINGS

FIGS. 1A and 1B shows data from different combinations of Olaparib and Drug X (candidate agent) at different concentrations against 10 Triple Negative Breast Cancer (TNBC) COMPASS1 tumoroids using COMPASS 1.

FIG. 2 are cell images of tumor volume 14 days post-treatment with Drug X and a variety of combinations of Drug X with other drugs using COMPASS 1 TNBC PDTs using COMPASS 1.

FIG. 3 are cell images of tumor volume using COMPASS 1.

FIG. 4 shows tumor volume reduction post treatment with Drug X, Drug Y, and combinations using COMPASS 1. The Y axis represents the normalized tumor volume fold change

FIG. 5 shows the mechanism of cell death upon treatment using COMPASS 1.

FIG. 6 shows if individual drug treatment or combination therapy directly affected cell proliferation using COMPASS 1. In this figure the Y axis is the Normalized Proliferation Rate.

FIGS. 7A and 7B show RNA Seq analysis of fold changes in significant genes comparing the effect of treatment with tumor vehicle vs. combination treatment using COMPASS 1.

FIG. 7C shows an ingenuity pathway analysis of genes common to T3 and T4 using COMPASS 1.

FIG. 8 shows the response in tumoroids to the addition of PBMCs using COMPASS 2.

FIG. 9 shows that adding PBMCs post-treatment with Drug X showed an increase in percentage of TILs in the COMPASS 2 platform using COMPASS 2.

FIGS. 10A-D shows tumor infiltration data using COMPASS 2.

FIG. 11 shows that using the COMPASS 2 platform, Drug X had increased TIIL incorporation over time.

FIG. 12 shows tumor infiltration data using COMPASS 2.

FIG. 13 shows data for the presence of immune components in immune competent carcinosarcoma tumoroids using COMPASS 2.

FIG. 14 shows data for the presence of immune components in immune competent carcinosarcoma tumoroids using COMPASS 2.

FIG. 15 shows data for percentage if different cell types.

FIG. 16 shows data for FACS-analysis of dissociated GBC-1 organoids co-cultured with PBMC collected at different time points.

FIGS. 17A-D show data for a quantitative live-dead assay using COMPASS 2.

FIGS. 18A-B show data for a FACS Analysis of Lymphocyte and Lymphocyte Subset in GBC-1 using COMPASS 2.

FIGS. 19A-B shows data for a FACS Analysis of Lymphocyte and Lymphocyte Subset in GBC-2 using COMPASS 2.

FIGS. 20A-B shows data for Treg cells co-cultured with PBMCs collected at different time points using COMPASS 2.

FIG. 20C shows data for CTLs co-cultured with PBMC collected at different time points using COMPASS 2.

FIG. 21 show modeling immunotherapy with co-culture of TNBC-1 tumoroids and CAR-T Cells using COMPASS 2.

FIG. 22 shows modeling immunotherapy with co-culture of TNBC-2 and CAR-T Cells using COMPASS 2.

FIG. 23 shows phase contrast images of TNBC-2 PDTs treated with CAR-T-Mock and EGFR for different time periods using COMPASS 2.

FIG. 24A shows a schematic of the various COMPASS 3 platforms.

FIGS. 24B-C shows cell images of static vs dynamic non-immunocompetent COMPASS 3 platforms representing various timepoints.

FIG. 25 shows static vs dynamic non-immunocompetent COMPASS 3 platform mean volume growth profiles.

FIG. 26 shows static vs dynamic immunocompetent COMPASS 3 platforms on day 3 pre-treatment

FIGS. 27A-F shows human uterine adenosarcoma growth profiles across variable COMPASS 3 platforms under variable oxygenation.

FIGS. 28A-L show common cancers (TNBC and GBC) vs established drug regimens treatment profiles across progressing, appropriately-matched COMPASS platforms at Day 3 and Day 10 timepoints. This study shows the differential drug response to each of the COMPASS model subtypes highlighting the functional significance of physiological oxygenation.

FIG. 29: are cell images of the impact of oxygen on tumor volume.

FIG. 30A: shows data of HSP90 Inhibitor was effective in reducing tumor volume using a COMPASS 1 model (without oxygen).

FIG. 30B: shows data of HSP90 Inhibitor was effective in reducing tumor volume using a COMPASS 3 model (with oxygen).

FIGS. 31A-C shows human uterine adenosarcoma (HUAS) vs HSP90i+/−Doxorubicin treatment profiles across progressing compass platforms at Day 3 & 7 Timepoints.

### DETAILED DESCRIPTION

### Definitions

As used herein, certain terms may have the following defined meanings.

As used in the specification and claims, the singular form “a,” “an” and “the” include singular and plural references unless the context clearly dictates otherwise. For example, the term “a cell” includes a single cell as well as a plurality of cells, including mixtures thereof.

“Allogeneic” refers to entities (e.g., cells, tumoroids, co-cultures) derived from different patients. In the case of cells, this can refer to cells derived from a sample from a different patient or healthy control.

“Approximately” or “about”, as used in this application, are equivalent. Any numerals used in this application with or without about/approximately are meant to cover any normal fluctuations appreciated by the person skilled in the art. As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Biologically active” refers to a characteristic of any agent that has activity in a biological system, and particularly in an organism. For instance, an agent that, when administered to an organism, has a biological effect on that organism, is considered to be biologically active.

The terms “cancer” and “cancerous” refer to or describe the physiological condition in mammals that is typically characterized by unregulated cell growth. Included in this definition are benign and malignant cancers. The term “cancer” is not limited to any stage, grade, histo morphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “cell culture” or “culture” means the maintenance of cells in an artificial, in vitro environment. It is to be understood, however, that the term “cell culture” is a generic term and are used to encompass the cultivation not only of individual cells, but also of tissues or organs.

The term “culture system” is used herein to refer to the culture conditions in which the subject explants are grown that promote prolonged tissue expansion with proliferation, multilineage differentiation and recapitulation of cellular and tissue ultrastructure.

“Co-culture” refers to two or more cell types maintained in conditions suitable for their mutual growth. In the context of the disclosure, an “organoid/tumoroid-immune cell co-culture” or “tumoroid-immune cell co-culture” relates to an organoid/tumoroid, as defined elsewhere, in culture with an immune cell type. In some embodiments, cell types in co-culture exhibit a structural, biochemical and/or phenomenological association that they do not exhibit in isolation. In some embodiments, cell types in co-culture mimic the structural, biochemical and/or phenomenological association observed between the cell types in vivo.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

The terms “comprises”, “comprising”, “includes”, “including”, “having” and their conjugates mean “including but not limited to”.

The term “consisting of” means “including and limited to”.

The term “consisting essentially of” means that the composition, method, or structure may include additional ingredients, steps and/or parts, but only if the additional ingredients, steps and/or parts do not materially alter the basic and novel characteristics of the claimed composition, method, or structure.

Compass 1 as used herein means a matrigel-based platform that forms a PDT/tumoroid-immune cell co-culture that comprises epithelium and stroma cells. In some embodiments, Compass 1 is non-scaffold based.

Compass 2 as used herein means a matrigel-based platform in which the PDT/tumoroid-immune cell co-culture comprises PBMCs, epithelium and stroma cells. This system can test functional immunocompetence, immunotherapies and is closer to an actual patient tumor. In some embodiments, Compass 2 is non-scaffold based. In some embodiments, Compass 2 is scaffold based.

Compass 3 as used herein means a matrigel-based platform in which the PDT/tumoroid-immune cell co-culture is co-cultured in a first media liquid-second media liquid interface. A Liquid-liquid interface is formed when the dissolved oxygen from the synthetic hemoglobin containing blood substitute interacts with tumoroid growth media. COMPASS 3 platforms can have various subversions: scaffold-based model: “3a” (−) Oxygenation [Static], (−) immunocompetence; “3b” (−) Oxygenation [Static], (+) Immunocompetence; “3c” (+) Oxygenation [Dynamic], (−) Immunocompetence; and “3d” (+) Oxygenation [Dynamic], (+) Immunocompetence. In some embodiments, Compass 3 is non-scaffold based. In some embodiments, Compass 3 is scaffold based. In some embodiments, Compass 3 is non-scaffold based and the media comprises oxygen. In some embodiments, Compass 3 is scaffold based and the media comprises oxygen. In the Compass 3 platform the PDT/tumoroid-immune cell co-culture can be co-cultured with or without PBMCs. This system allows for physiological flow for oxygen. As such, oxygen levels in the PDT are equivalent what is found in human blood-making it physiologically equivalent/relevant flow of oxygen in the system. Flow rates of the PDT/system are generally equal to the interstitial flow rates of the corresponding organ for advanced modeling of the physiological cell environment. Since the system is organ agnostic the system is designed to have different physiologically equivalence depending on the organ being tested. This system can test functional immunocompetence, immunotherapies, therapies influenced by pH modulation and is closest to an actual patient tumor. In summary, and generally speaking, COMPASS 1 has stroma and epithelium; COMPASS 2 has stroma, epithelium and immune components; and finally, COMPASS 3 has stroma, epithelium, immune components, and oxygenation. By “container” is meant a glass, plastic, or metal vessel that can provide an aseptic environment for culturing cells.

The term “candidate cells” refers to any type of cell that is placed in co-culture with the tissue explants described herein. Candidate cells include without limitations, genetically engineered T cells including without limitation CAR-T cells, dendritic cells, phagocytic cells T cells, B cells, etc. The term “candidate agent” means any oligonucleotide, polynucleotide, siRNA, shRNA, gene, gene product, peptide, antibody, small molecule, or pharmacological compound that is introduced to an explant culture and the cells thereof as described herein to assay for its effect on the explants. As used herein Candidate agent includes candidate cells.

The term “contacting” refers to the placing of candidate cells or candidate agents into the explant culture as described herein. Contacting also encompasses co-culture of candidate cells with tissue explants for at least 1 hour, or more than 2 hrs or more than 4 hrs in culture medium prior to placing the tissue explants in a semi-permeable substrate. Alternatively, contacting refers to injection of candidate cells into the explant, e.g., into the lumen of an explant.

The term “co-cultured” as used herein means the tumoroid-immune cell co-culture of at least one non-immune cell and at least one immune cell.

The term “diagnosis” is used herein to refer to the identification of a molecular or pathological state, disease, or condition, such as the identification of a molecular subtype of breast cancer, prostate cancer, or other type of cancer.

“Dose” refers to a specified quantity of a pharmaceutical agent provided in a single administration. In certain embodiments, a dose is administered in two or more boluses, tablets, or injections. For example, in certain embodiments, where subcutaneous administration is desired, the desired dose requires a volume not easily accommodated by a single injection. In such embodiments, two or more injections are used to achieve the desired dose. In certain embodiments, a dose is administered in two or more injections to minimize injection site reaction in an individual. In certain embodiments, a dose is administered as a slow infusion.

The term “explant” is used herein to mean a piece of tumor tissue and the cells thereof originating from the tumor tissue that is cultured in vitro, for example according to the methods of the invention. The tissue from which the explant is derived is obtained from an individual, i.e., a cancer patient. Methods of interest include patient-specific analysis of anti-tumor immune responses.

The term “functional immunocompetence” means the tumoroid/PDTs ability to resist and control infections. Various methods are used for evaluating immunocompetence which are known to those of skill in the art. As used herein a “functional immunocompetent PDT” and the like comprises a mononuclear cell, a lymphocyte, a monocyte, a peripheral blood mononuclear cell (PBMC), T Cells (CD3+), B cells (CD20), Macrophages (CD68), NK cells (CD16), and/or a combination thereof.

As used herein, the term “immune cell” includes cells that are of hematopoietic origin and that play a role in the immune response. Immune cells include lymphocytes, such as B cells and T cells; natural killer cells; myeloid cells, such as monocytes, macrophages, eosinophils, mast cells, basophils, and granulocytes.

“Immune disease” refers to any disorder of the immune system. Immune diseases typically have a genetic component and include autoimmune diseases (in which the immune system erroneously acts upon ‘self’ components) and immune-mediated diseases (in which the immune system exhibits excessive function).

“Immunotherapy” refers to any medical intervention that induces, suppresses, or enhances the immune system of a patient for the treatment of a disease. In some embodiments, immunotherapies activate a patient's innate and/or adaptive immune responses (e.g., T cells) to more effectively target and remove a pathogen or cure a disease, such as cancer or an immune disease.

“Physiological oxygenation” refers to a cell culture where synthetic hemoglobin is added at the base of the PDT in a very viscous liquid which does not mix with media (forming a liquid-liquid interface) but can circulate oxygen to the tumoroids/PDT in the same manner as the human system using the physiological pump

“Organoid” refers to a cellular structure obtained by expansion of adult (post-embryonic) epithelial stem cells, preferably characterized by Lgr5 expression, and consisting of tissue-specific cell types that self-organize through cell sorting and spatially restricted lineage commitment (e.g., as described in Clevers, Cell. 2016 Jun. 16; 165 (7): 1586-1597, see particularly section called “Organoids derived from adult stem cells” at page 1590 onwards). Herein, the term “organoid” is used to refer to normal (e.g., non-tumor) organoids. Where organoids are described as “disease” organoids, this means that the organoid has a disease phenotype, e.g., typically because the organoid has been derived from one or more epithelial stem cell having a disease phenotype, or in some embodiments, because the organoid has been genetically modified to display particular characteristics of a disease phenotype.

As used herein “patient-derived tumoroid” or PDT as used herein means a tumoroid-immune cell co-cultured with immune cells in media including oxygen (unless context suggests otherwise).

Peripheral blood mononuclear cells (PBMCs) means peripheral blood mononuclear cells (PBMCs) are isolated from peripheral blood and identified as any blood cell with a round nucleus (i.e., lymphocytes, monocytes, natural killer cells (NK cells) or dendritic cells).

A “gas-liquid interface” is the interface to which the cell culture media is put in a cell culture incubator with 95% air and 5% CO2 and the gas and liquid interact with each other to oxygenate the tumors while they grow. In a gas-liquid interface ambient oxygen concentration cannot be controlled/modulated.

An “Liquid-liquid interface” is the interface to which oxygen interacts with the liquid media directly as it is captured by a synthetic hemoglobin and dissolved oxygen is taken up by the liquid media which, in a closed system, feeds media with dissolved oxygen to the tumoroid during growth.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence. The present methods allow prediction of whether a patient will be responsive to a therapy of interest.

“PDT response” means any change to the PDT (positive or negative) which is the result of an external or internal stimulus (such as the candidate agent).

“Patient response” means any change to the patient (positive or negative) which is the result of an external or internal stimulus and is assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesion size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

“Population” refers to a group of entities sharing common traits. In some embodiments, “population” refers to patients sharing a set of relevant clinical traits. Preferably, a “population” may refer to a group of patients sharing the same cancer, and/or being treated with the same agent, and/or susceptible to successful treatment with the same agent. A population may differ in one or more characteristics, including genotype and/or specific agent response characteristics during treatment. A population may also refer to a group of cells, organoids, and/or co-cultures sharing one or more genotypic, phenotypic, or biochemical traits. A “sub-population” refers to a group of entities sharing a greater number of common traits, or a smaller number of dissimilar traits, than a larger population in which the entities of the sub-population are also classified.

“Safe” refers to a treatment for a disease, which has no side-effects or only has side-effects within a tolerable level according to standard clinical practice.

“Side effect” or “deleterious effect” refers to a physiological response attributable to a treatment other than desired effects.

“Screening” refers to the process of either co-culturing candidate cells with or adding candidate agents to the PDT culture described herein and assessing the effect of the candidate cells or candidate agents on the PDT, including without limitation immune cells present in the PDT. The effect is assessed by assessing any convenient parameter, e.g., phenotypic changes, protein expression, mRNA expression, etc.

“Subject” or “patient” or “individual” may refer to a human or any non-human animal (such as any mouse, rat, rabbit, dog, cat, cattle, swine, sheep, horse, or primate). In preferred embodiments, the patient is a mammal, more preferably a human. “Human” may refer to pre- and/or post-natal forms. A subject is a patient, which refers to a human presenting to a medical provider for diagnosis or treatment of a disease. The term “subject” is used herein interchangeably with “individual” or “patient.” A patient is afflicted with or is susceptible to a disease or disorder but may or may not display symptoms of the disease or disorder.

“Suffering from” refers to a patient who has been diagnosed with or displays one or more symptoms of a disease, disorder, and/or condition.

“Susceptible to” refers to a patient who has not been diagnosed with a disease, disorder, and/or condition. In some embodiments, a patient who is susceptible to a disease, disorder, and/or condition may not exhibit symptoms of the disease, disorder, and/or condition. In some embodiments, a patient who is susceptible to a disease, disorder, condition, or event are characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or (5) having undergone, planning to undergo, or requiring a transplant. In some embodiments, a patient who is susceptible to a disease, disorder, and/or condition will develop the disease, disorder, and/or condition. In some embodiments, a patient who is susceptible to a disease, disorder, and/or condition will not develop the disease, disorder, and/or condition.

The term “small molecule” refers to an organic molecule having a molecular weight between 50 Daltons to 2500 Daltons.

By “standard of care” herein is intended the anti-tumor/anti-cancer agent or agents that are routinely used to treat a particular form of cancer.

The terms “therapeutically effective amount” or “effective amount” refer to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it is cytostatic and/or cytotoxic. The effective amount may extend progression free survival (e.g., as measured by Response Evaluation Criteria for Solid Tumors, RECIST, or CA-125 changes), result in an objective response (including a partial response, PR, or complete response, CR), improve survival (including overall survival and progression free survival) and/or improve one or more symptoms of cancer (e.g., as assessed by FOSI). Most preferably, the therapeutically effective amount of the drug is effective to improve progression free survival (PFS) and/or overall survival (OS).

“Treating”, “treat”, “treatment” refers to any method used to partially or completely alleviate, ameliorate, relieve, inhibit, prevent, delay onset of, reduce severity of and/or reduce incidence of one or more symptoms or features of a particular disease, disorder, and/or condition. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, methods and compositions of the invention are useful in attempts to delay development of a disease or disorder. Treatment is administered to a subject who does not exhibit signs of a disease and/or exhibits only early signs of the disease for the purpose of decreasing the risk of developing pathology associated with the disease.

“Tumoroid” refers to an organoid comprising, consisting of, or consisting essentially of cells which exhibit one or more genetic, phenotypic, or biochemical traits classified as cancerous. Herein, the term “tumoroid” encompasses “organoids” derived from cancerous tissue. The term “tumoroid” may also encompass genetically engineered organoids.

“Tumor immune microenvironment” (TME) means a host of immunosuppressive factors that allow the tumor cells to keep the resident T cells in check and to evade anti-tumor immunity. Despite intensive research, current immunotherapy approaches have yielded disappointing results. Recent work suggests that the body can generate an antitumor immune response, yet only marginal improvements in survival have been observed. One reason for the disappointing results is the tumor immune microenvironment. Tumor cells have devised a complex set of mechanisms to evade the immune response. Cancers can mute an immune response through several mechanisms, which include, but are not limited to downregulating the expression of the major histocompatibility complex (MHC), increasing activation of regulatory T cells (Treg), and expressing an immunosuppressive cytokine profile.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

“Ultrastructure” refers to the three-dimensional structure of a cell or tissue observed in vivo. For example, the ultrastructure of a cell is its polarity or its morphology in vivo, Whilst the ultrastructure of a tissue would be the arrangement of different cell types relative to one another within a tissue.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the rang.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

Unless otherwise defined, all technical and/or scientific terms used herein have the same meaning as commonly understood by one of ordinary skill in the art. Although methods and materials similar or equivalent to those described herein can be used in the practice or testing of embodiments disclosed herein, exemplary methods and/or materials are described below. In case of conflict, the patent specification, including definitions, will control. In addition, the materials, methods, and examples are illustrative only and are not intended to be necessarily limiting.

While some embodiments comprise/include the disclosed features and may therefore include additional features not specifically described, other embodiments may be essentially free of or completely free of non-disclosed elements—that is, non-disclosed elements may optionally be essentially omitted or completely omitted.

Before explaining at least one embodiment in detail, it is to be understood that the disclosure is not necessarily limited in its application to the details set forth in the following description or exemplified by the Examples. The disclosure is capable of other embodiments or of being practiced or carried out in various ways.

### Tumoroid Formation

### Cell Types for Tumoroid Formation

In some embodiments, tissue is sourced from commercial vendors or tissue sharing partners, and can be live or cryopreserved, and can include pathology report or treatment profile.

Organoids, tumoroids, and/or co-cultures of the disclosure are suitable for use with any multicellular organism, preferably a multicellular organism susceptible to cancer. In some embodiments, the organoids, tumoroids, and/or co-cultures of the disclosure are mammalian (meaning derived from mammals), such as murine, mammal, primate or human cells, cancers, organoids, tumoroids, and/or co-cultures. In some embodiments, the cells, cancers, organoids, tumoroids, and/or co-cultures of the disclosure are human (meaning derived from humans).

In some embodiments, the organoids/tumoroids and/or organoid/tumoroid-immune cell co-cultures of the disclosure are obtained from epithelial cells. In some embodiments, the organoids/tumoroids and/or organoid/tumoroid-immune cell co-cultures of the disclosure are not obtained from epithelial cells. In some embodiments, the organoids/tumoroids and/or organoid/tumoroid-immune cell co-cultures of the disclosure are obtained from normal (i.e., non-disease) cells or from disease cells (tumoroids). In some embodiments, tumoroids and/or tumoroid-immune cell co-cultures of the disclosure are obtained from tumor epithelial cells. In some embodiments, tumoroids and/or tumoroid-immune cell co-cultures of the disclosure are not obtained from tumor epithelial cells. In some embodiments, the immune cells in the organoid/tumoroid-immune cell co-cultures of the disclosure are obtained from normal (i.e., non-disease) cells.

In some embodiments, tumor cells and/or normal cells include lung cells, liver cells, breast cells, skin cells, intestinal cells, crypt cells, rectal cells, pancreatic cells, endocrine cells, exocrine cells, ductal cells, renal cells, adrenal cells, thyroid cells, pituitary cells, parathyroid cells, prostate cells, stomach cells, oesophageal cells, ovary cells, fallopian tube cells and vaginal cells. In some embodiments, epithelial cells are intestinal cells, for example colorectal cells.

In some embodiments, the tumor cells and/or normal cells are obtained from a sample from a cancer patient. In some embodiments, tumor cells and normal cells are obtained from samples from the same cancer patient. In some embodiments, tumor cells and normal cells are obtained from samples from the same cancer patient, from the same sample. In some embodiments, tumor cells and normal cells are obtained from samples from different patients. Suitable samples for obtaining cells include tissue biopsy, such as ascites from a colorectal or ovarian cancer patient; urine from a kidney cancer patient; or tissue biopsy from resected colon and/or rectum of colorectal cancer patient.

Tumor tissue are obtained by any convenient method, e.g., by biopsy, e.g., during endoscopy, during surgery, by needle, etc., and is typically obtained as aseptically as possible. In some embodiments, organoid and/or tumoroid samples are obtained during surgery from normal mucosa and tumor tissue, for example taken from resected colon, rectum, small intestine and/or ileum of colorectal cancer patients and/or healthy control patients. In some embodiments, the immune cells are derived from peripheral blood taken during surgery. In some embodiments, the immune cells are derived from the tumor biopsy.

In some embodiments, the PDT are, for example, derived from a tissue resection, a tissue biopsy, or a metastatic lesion obtained from a patient suffering from, suspected to suffer from, or diagnosed with cancer. In some embodiments, the sample is a sample of a tissue, a resection or biopsy of a tumor, a known or suspected metastatic cancer lesion or section, or a blood sample, e.g., a peripheral blood sample, known or suspected to comprise circulating cancer cells. In some embodiments, the sample may comprise both cancer cells, i.e., tumor cells, and non-cancerous cells, and, in certain embodiments, comprises both cancerous and non-cancerous cells. In some embodiments, the sample may comprise both cancer cells, i.e., tumor cells, and non-cancerous cells, and, in certain embodiments, comprises both cancerous and non-cancerous cells as well as at least one immune cell. In some embodiments, the PDT of the disclosure comprises engineered T cells.

In some embodiments, any immune cell that is incorporated into a co-culture is suitable for use with methods of the disclosure. Preferred immune cells include one or more cell types selected from the group consisting of intra-epithelial lymphocytes (IELs), tumor infiltrating lymphocytes (TILs), peripheral blood mononuclear cells (PBMCs), peripheral blood lymphocytes (PBLs), T cells, cytotoxic T lymphocytes (CTLs), B cells, NK cells, mononuclear phagocytes, α/β receptor T-cells and γ/δ receptor T cells.

In some embodiments, the immune cells incorporated into the PDT are obtained from established cell lines available in the art (e.g., from ATCC or similar libraries of cell lines). In some embodiments, the immune cells are purified from a sample from a subject from which the tumor sample is taken. In some embodiments, the immune cells are part of the tumor biopsy and are not separately collected.

In some embodiments, the immune cells are obtained from a peripheral blood sample and/or a tissue biopsy. For example, peripheral blood lymphocytes (PBLs) and/or T cells are obtained from a peripheral blood sample; or tumor-infiltrating lymphocytes (TILs) and/or intra-epithelial lymphocytes (IELs) are obtained from the tumor or healthy tissue biopsy, respectively.

In some embodiments, the immune cells suitable for use in methods of the disclosure are allogeneic with the tumoroid and/or organoid. In some embodiments, the immune cells are HLA-matched with the tumoroid and/or organoid, that is, the immune cells are antigenically compatible with the patient from whom the tumoroid and/or organoid are derived. In some embodiments, the immune cells suitable for use in methods of the disclosure are autologous with the tumoroid and/or organoid.

Tumoroids and/or organoids of the disclosure may comprise or consist of autologous cells, i.e., cells obtained from the same patient. For example, the tumoroid are obtained by culturing a tumor cell (e.g., a colorectal cancer cell), whereas the organoid is obtained by culturing a normal (non-tumor) cell from the same tissue in the same patient (e.g., a normal colon cell). This is particularly useful in the context of a reference organoid.

Tumoroids and/or organoids of the disclosure may comprise or consist of allogeneic cells, i.e., cells obtained from a different patient. For example, the tumoroid are obtained by culturing a tumor cell (e.g., a colorectal cancer cell), whereas the organoid is obtained by culturing a normal (non-tumor) cell from the same tissue from a different patient (e.g., a normal colon cell).

In some embodiments, tumoroids and/or organoids are separated into populations sharing one or more genotypes, phenotypes, and/or epigenetic markers.

In some embodiments, the PDT comprises engineered T cells, such as chimeric antigen receptor (CAR)-T cells. Disclosed are methods and co-cultures which are used for testing the suitability of different CAR-T cell types for different tumor phenotypes and tumor microenvironments. Disclosed is a method for streamlining the process of CAR-T cell selection and performance augmentation, with improved scalability and reduced cost compared to existing methods.

In some embodiments, the organoids are prepared by culturing normal epithelial cells in an organoid culture medium. In some embodiments, the tumoroids are prepared by culturing tumor epithelial cells in a tumoroid culture medium. In some embodiments, the tumor cell and immune cell are autologous i.e., from the same patient. In some embodiments, the tumor cell and immune cell are not from the same patient. In some embodiments, the organoid/tumoroid is a three-dimensional cellular structure. In some embodiments, the organoid/tumoroid comprises a lumen surrounded by epithelial cells. In some embodiments, the epithelial cells surrounding the lumen are polarized. The polarization is disrupted in tumoroids.

### Method for Tumoroid Formation

Tumoroid formation begins with a tumor sample. As discussed more fully above.

In some embodiments, upon removal, tumor tissue is immersed in ice-cold buffered solution, e.g., PBS, Ham's F12, MEM, culture medium, etc. In some embodiments, pieces of tissue are minced to a size less than about 1 mm3, and are less than about 0.5 mm3, or less than about 0.1 mm3. In some embodiments, the minced tissue is mixed with a media. In some embodiments, the tumoroid media is supplied externally with oxygen. In some embodiments, the oxygen is supplied externally to the tumoroid generating media via a gas-liquid interface. In some embodiments, gas forming the gas-liquid interface has a lower oxygen concentration than ambient air. In some embodiments, gas forming the gas-liquid interface has a higher oxygen concentration than ambient air. In some embodiments, gas forming the gas-liquid interface includes from about 0% to about 90% of oxygen. In some embodiments, at least a portion of the tumoroid generating media is replaced. In some embodiments, at least a portion of the tumoroid generating media is infused into a system containing the construct of cells for a first predetermined period of time. In some embodiments, at least a portion of the tumoroid generating media is withdrawn from the system for a second predetermined period of time wherein the first period of time and second period of time are the same or are different. In some embodiments, the first period of time is between 1-60 days and the second period of time is between 1-60 days. In some embodiments, the tumoroid generating media includes a blood substitute. In some embodiments, the tumoroid generating media includes hemoglobin. In some embodiments, the tumoroid generating media includes synthetic hemoglobin. In some embodiments, the tumoroid generating media comprises a first tumoroid generating media and a second tumoroid generating media different from the first tumoroid generating media (i.e., the first tumoroid generating media is removed and the second tumoroid generating media is added or the second tumoroid generating media is added to the first tumoroid generating media). In some embodiments, the second tumoroid generating media includes blood substitute. In some embodiments, the second tumoroid generating media includes hemoglobin. In some embodiments, the second tumoroid generating media includes artificial hemoglobin. In some embodiments, the density of the first tumoroid generating media is lower than the second tumoroid generating media. In some embodiments, the density of the first tumoroid generating media is higher than the second tumoroid generating media. In some embodiments, the density of the first tumoroid generating media is the same as the density of the second tumoroid generating media. In some embodiments, the second tumoroid generating media forms a liquid-liquid interface with the first tumoroid generating media. In some embodiments, the concentration of oxygen in the first tumoroid generating media is from about 0 μM to about 300 μM. In some embodiments, the concentration of oxygen in the second tumoroid generating media is from about 0 μM to about 300 μM. In some embodiments, the concentration of oxygen in the first tumoroid generating media is higher than the concentration of oxygen in the second tumoroid generating media. In some embodiments, the concentration of oxygen in the first tumoroid generating media is lower than the concentration of oxygen in the second tumoroid generating media. In some embodiments, the concentration of oxygen in the first tumoroid generating media is the same as the concentration of oxygen in the second tumoroid generating media. In some embodiments, the concentration of oxygen in the first tumoroid generating media is 0 μM and the concentration of oxygen in the second tumoroid generating media is from about 20 μM to about 300 μM. In some embodiments, the concentration of oxygen in the first tumoroid generating media is from about 20 μM to about 300 M and the concentration of oxygen in the second tumoroid generating media is 0 μM. In some embodiments, the concentration of oxygen in the first tumoroid generating media is from about 20 μM to about 300 μM and the concentration of oxygen in the second tumoroid generating media is from about 20 μM to about 300 μM. In some embodiments, the tumoroid is generated without immune cells and then immune cells are added once the tumoroid is formed. In some embodiments, the tumoroid is formed in the presence of immune cells.

In some embodiments, the tumoroid generating media comprises exogenous entactin. In some embodiments, the tumoroid generating media comprises Matrigel™. In some embodiments, the tumoroid generating media induces more cell proliferation of the construct of cells within a given period of time (i.e., about 1-10, 1-20, 1-30, 1-40, 1-50, or 1-100 days). In some embodiments, the tumoroid generating media induces less cell proliferation of the construct of cells within a given period of time (i.e., about 1-10, 1-20, 1-30, 1-40, 1-50, or 1-100 days). In some embodiments, the tumoroid generating media induces more mass of the construct of cells within a given period of time. In some embodiments, the tumoroid generating media induces more volume of the construct of cells within a given period of time. In some embodiments, the tumoroid generating media includes a three-dimensional (3D) structure. In some embodiments, the tissue forms a three-dimensional (3D) patient-derived tumoroid (PDTs). In some embodiments, the tissue derived from the patient includes cancer cells and/or immune cells. In some embodiments, the tissue derived from the patient does not include cancer cells and/or immune cells. In some embodiments, the tissue derived from the patient includes sarcoma cells, gallbladder cancer, and/or Human Uterine Adenosarcoma cells. In some embodiments, the tissue derived from the patient does not include sarcoma cells, gallbladder cancer, and/or Human Uterine Adenosarcoma cells.

The arrangement described above allows oxygen to travel throughout the PDT. In some embodiments, the tumoroid generating media covers the PDT. In some embodiments, the tumoroid generating media does not cover the PDT. In some embodiments, the tumoroid generating media partially covers the PDT. In some embodiments, the tumoroid generating media surrounds covers the PDT. In some embodiments, the first tumoroid generating media is exposed to air. In some embodiments, the second tumoroid generating media is exposed to air. In some embodiments, the PDT is grown at a liquid-liquid interface of the first and second tumoroid generating media. In some embodiments, the PDT is grown at the first tumoroid generating media. In some embodiments, the PDT is grown the second tumoroid generating media. In some embodiments, the PDT is exposed to air. In some embodiments, the PDT is not exposed to air. In some embodiments, the PDT is partially exposed to air.

In some embodiments, organoids/tumoroids are cultured in human organoid medium. In some embodiments, the organoids/tumoroids are passaged every 14-30 days. In some embodiments, the organoids/tumoroids are passaged every 14-30 days by dissociation with 200 units ml-1 collagenase IV (Worthington) at 37° C. for 30 min, followed by three 5-min washes with 100% FBS and replating. Experimental modifications are made by any method known in the art to the tumoroid generating media.

Methods disclosed herein are performed in vivo, ex vivo, in vitro, in situ, ex situ, or any combination thereof. Preferably the methods are performed in vitro.

In some embodiments, the tumor tissue is from a human specimens. In some embodiments, the tumor tissues are obtained from a tissue bank. In some embodiments, the tumor tissues are obtained from a patient.

In some embodiments, tumoroid culture media and organoid culture media are used to prepare organoids and tumoroids for co-culture, for example, by facilitating growth, division (expansion), structural organization, or other development to produce a tumoroid and/or organoid suitable for co-culture. Suitable tumoroid culture media and organoid culture media for different tissues are known in the art (e.g., Clevers, Cell. 2016 Jun. 16; 165 (7): 1586-1597).

### Immune Cell Culture Formation.

Immune cells are derived from a sample and culture in culture media. Immune cell culture medium is used to prepare immune cells for co-culturing, for example, by facilitating growth and division (expansion) and/or differentiation of immune cells to produce a population suitable for co-culture with a tumor cell/tumoroid. In some embodiments, the immune cell culture medium comprises an interleukin. In some embodiments the interleukin is selected from IL-2, IL-7, and IL-15. In some embodiments, the interleukin in the immune cell culture medium is IL-2. In some embodiments the concentration of the interleukin 2000-6000 IU/mL. A preferred concentration of IL-2 in the immune cell culture medium is 50 μM. The immune cell culture medium may further comprise an RPMI medium (e.g., RPMI 1640, Gibco), optionally supplemented with penicillin/streptomycin and/or hepes and/or glutaMAX™ and/or sodium pyruvate and/or serum (e.g., 5% human AB serum, Sigma-Aldrich). In principle, any mammalian basal cell culture medium is used in place of RPMI medium, such as DMEM/12.

### Tumoroid-Immune Cell Co-Culture Formation.

Provided is a method for preparing a tumoroid-immune cell co-culture, wherein the method comprises: culturing patient derived tumor cells and patient derived immune cells in a first tumoroid culture medium that does not comprise oxygen to obtain a tumoroid; removing the first tumoroid culture medium from the tumoroid; resuspending the tumoroid and at least one immune cell in a second tumoroid culture medium comprising, consisting of, or consisting essentially of oxygen thereby forming the tumoroid-immune cell co-culture. In some embodiments, the first tumoroid culture medium comprises oxygen.

Provided is a method for preparing a tumoroid-immune cell co-culture, wherein the method comprises: culturing patient derived tumor cells and patient derived immune cells in a first tumoroid culture medium that does not comprise oxygen to obtain a tumoroid; adding to the tumoroid a second tumoroid culture medium comprising, consisting of, or consisting essentially of oxygen thereby forming the tumoroid-immune cell co-culture.

In some embodiments, the disclosure provides a method for preparing a tumoroid-immune cell co-culture. In some embodiments, tumoroid-immune cell co-culture is cultured in media supplied externally with oxygen. In some embodiments, the oxygen is supplied externally to the media via a gas-liquid interface. In some embodiments, gas forming the gas-liquid interface has a lower oxygen concentration than ambient air. In some embodiments, gas forming the gas-liquid interface has a higher oxygen concentration than ambient air. In some embodiments, gas forming the gas-liquid interface includes from about 0% to about 90% of oxygen. In some embodiments, the media comprises a first media and a second media different from the first media (i.e., the first media is removed, and the second media is added, or the second media is added to the first media). In some embodiments, the density of the first media is lower than the second media. In some embodiments, the density of the first media is higher than the second media. In some embodiments, the density of the first media is the same as the density of the second media. In some embodiments, the second media forms a liquid-liquid interface with the first media. In some embodiments, the concentration of oxygen in the first media is from about 0 μM to about 300 μM. In some embodiments, the concentration of oxygen in the second media is from about 0 μM to about 300 μM. In some embodiments, the concentration of oxygen in the first media is 0 μM and the concentration of oxygen in the second media is from about 20 μM to about 300 M. In some embodiments, the concentration of oxygen in the first media is from about 20 μM to about 300 μM and the concentration of oxygen in the second media is 0 μM. In some embodiments, the concentration of oxygen in the first media is from about 0 μM to about 300 μM and the concentration of oxygen in the second media is from about 0 μM to about 300 μM.

In some embodiments, an immune cell (taken from the patient or from a different source such as a tissue bank) is mixed with the non-immune cells in an in vitro culture. Mixing may comprise sequential layering of immune cells and non-immune cells (such as tumor cells or organoids/tumoroids) to the same container (such as a well in a multi-well plate) or may comprise sequential pipetting of immune cells and non-immune cell (such as tumor cells or organoids/tumoroids) into an oxygen containing media as described herein.

In some embodiments, the method for preparing the tumoroid-immune cell co-culture further comprises one or more of the following preparation steps: preparing the at least one tumor cell/tumoroid by culturing tumor cells in a tumoroid culture medium; and/or preparing the immune cells by culturing the immune cells in an immune cell expansion medium. Then combining the cultured tumoroid cells and immune cells in a tumoroid-immune cell co-culture wherein the tumoroid-immune cell co-culture is supplied with oxygen from a media including oxygen.

In some embodiments, the tumoroid culture medium (which in some cases includes an extracellular matrix) is removed from the at least one tumoroid before mixing the at least one tumoroid with the immune cells. Extracellular matrix is disrupted using commercially available kits, such as Cell Recovery Solution™ (Corning). In some embodiments, collagen is used instead of a matrix. In some embodiments, the tumoroid culture media does not include a matrix (matrix free).

In some embodiments, the tumoroid-immune cell co-culture media is supplied externally with oxygen. In some embodiments, the oxygen is supplied externally to the tumoroid-immune cell co-culture media via a gas-liquid interface. In some embodiments, gas forming the gas-liquid interface has a lower oxygen concentration than ambient air. In some embodiments, gas forming the gas-liquid interface has a higher oxygen concentration than ambient air. In some embodiments, gas forming the gas-liquid interface includes from about 0% to about 90% of oxygen. In some embodiments, at least a portion of the tumoroid-immune cell co-culture media is replaced. In some embodiments, at least a portion of the tumoroid-immune cell co-culture media is infused into a system containing the construct of cells for a first predetermined period of time. In some embodiments, at least a portion of the tumoroid-immune cell co-culture media is withdrawn from the system for a second predetermined period of time wherein the first period of time and second period of time are the same or are different. In some embodiments, the first period of time is between 1-60 days and the second period of time is between 1-60 days. In some embodiments, the tumoroid-immune cell co-culture media includes a blood substitute. In some embodiments, the tumoroid-immune cell co-culture media includes hemoglobin. In some embodiments, the tumoroid-immune cell co-culture media includes synthetic hemoglobin. In some embodiments, the tumoroid-immune cell co-culture media comprises a first tumoroid-immune cell co-culture media and a second tumoroid-immune cell co-culture media different from the first tumoroid-immune cell co-culture media (i.e., the first tumoroid-immune cell co-culture media is removed and the second tumoroid-immune cell co-culture media is added or the second tumoroid-immune cell co-culture media is added to the first tumoroid-immune cell co-culture media). In some embodiments, the second tumoroid-immune cell co-culture media includes blood substitute. In some embodiments, the second tumoroid-immune cell co-culture media includes hemoglobin. In some embodiments, the second tumoroid-immune cell co-culture media includes artificial hemoglobin. In some embodiments, the density of the first tumoroid-immune cell co-culture media is lower than the second tumoroid-immune cell co-culture media. In some embodiments, the density of the first tumoroid-immune cell co-culture media is higher than the second tumoroid-immune cell co-culture media. In some embodiments, the density of the first tumoroid-immune cell co-culture media is the same as the density of the second tumoroid-immune cell co-culture media. In some embodiments, the second tumoroid-immune cell co-culture media forms a liquid-liquid interface with the first tumoroid-immune cell co-culture media. In some embodiments, the concentration of oxygen in the first tumoroid-immune cell co-culture media is from about 0 μM to about 300 μM. In some embodiments, the concentration of oxygen in the second tumoroid-immune cell co-culture media is from about 0 μM to about 300 μM. In some embodiments, the concentration of oxygen in the first tumoroid-immune cell co-culture media is higher than the concentration of oxygen in the second tumoroid-immune cell co-culture media. In some embodiments, the concentration of oxygen in the first tumoroid-immune cell co-culture media is lower than the concentration of oxygen in the second tumoroid-immune cell co-culture media. In some embodiments, the concentration of oxygen in the first tumoroid-immune cell co-culture media is the same as the concentration of oxygen in the second tumoroid-immune cell co-culture media. In some embodiments, the concentration of oxygen in the first tumoroid-immune cell co-culture media is 0 μM and the concentration of oxygen in the second tumoroid-immune cell co-culture media is from about 20 μM to about 300 μM. In some embodiments, the concentration of oxygen in the first tumoroid-immune cell co-culture media is from about 20 μM to about 300 μM and the concentration of oxygen in the second tumoroid-immune cell co-culture media is 0 μM. In some embodiments, the concentration of oxygen in the first tumoroid-immune cell co-culture media is from about 20 μM to about 300 μM and the concentration of oxygen in the second tumoroid-immune cell co-culture media is from about 20 μM to about 300 μM. In some embodiments, the tumoroid is generated without immune cells and then immune cells are added once the tumoroid is formed. In some embodiments, the tumoroid is formed in the presence of immune cells.

In some embodiments, the tumoroid-immune cell co-culture media comprises exogenous entactin. In some embodiments, the tumoroid-immune cell co-culture media comprises Matrigel™ In some embodiments, the tumoroid-immune cell co-culture media induces more cell proliferation of the construct of cells within a given period of time (i.e., about 1-10, 1-20, 1-30, 1-40, 1-50, or 1-100 days). In some embodiments, the tumoroid-immune cell co-culture media induces less cell proliferation of the construct of cells within a given period of time (i.e., about 1-10, 1-20, 1-30, 1-40, 1-50, or 1-100 days). In some embodiments, the tumoroid-immune cell co-culture media induces more mass of the construct of cells within a given period of time. In some embodiments, the tumoroid-immune cell co-culture media induces more volume of the construct of cells within a given period of time. In some embodiments, the tumoroid-immune cell co-culture media includes a three-dimensional (3D) structure. In some embodiments, the tissue forms a three-dimensional (3D) patient-derived tumoroid (PDTs). In some embodiments, the tissue derived from the patient includes cancer cells and/or immune cells. In some embodiments, the tissue derived from the patient does not include cancer cells and/or immune cells. In some embodiments, the tissue derived from the patient includes sarcoma cells, gallbladder cancer, and/or Human Uterine Adenosarcoma cells. In some embodiments, the tissue derived from the patient does not include sarcoma cells, gallbladder cancer, and/or Human Uterine Adenosarcoma cells.

The arrangement described above allows oxygen to travel throughout the PDT-immune cell co-culture. In some embodiments, the tumoroid-immune cell co-culture media covers the PDT-immune cell co-culture. In some embodiments, the tumoroid-immune cell co-culture media does not cover the PDT-immune cell co-culture. In some embodiments, the tumoroid-immune cell co-culture media partially covers the PDT-immune cell co-culture. In some embodiments, the tumoroid-immune cell co-culture media surrounds covers the PDT-immune cell co-culture. In some embodiments, the first tumoroid-immune cell co-culture media is exposed to air. In some embodiments, the second tumoroid-immune cell co-culture media is exposed to air. In some embodiments, the PDT-immune cell co-culture is grown at a liquid-liquid interface of the first and second tumoroid-immune cell co-culture media. In some embodiments, the PDT-immune cell co-culture is grown at the first tumoroid-immune cell co-culture media. In some embodiments, the PDT-immune cell co-culture is grown the second tumoroid-immune cell co-culture media. In some embodiments, the PDT-immune cell co-culture is exposed to air. In some embodiments, the PDT-immune cell co-culture is not exposed to air. In some embodiments, the PDT-immune cell co-culture is partially exposed to air.

In some embodiments, the method further comprises the step of obtaining the immune cells and tumor cells from a patient sample. Methods for isolating immune cells from a sample are known in the art.

The disclosure provides a tumoroid-immune cell co-culture obtained by the above method. The disclosure also provides uses of said tumoroid-immune cell co-culture in drug screening, toxicology screening, research, drug development, and patient treatment.

The tumoroid-immune cell co-culture are ex situ, ex vivo, and/or in vitro. It is preferably in vitro.

In some embodiments, the organoid/tumoroid-immune cell co-cultures are moved into other formats such as multi-wells for screening or in submerged 2D or 3D geometries. In some embodiments, the candidate agent is placed underneath the PDT comprising organoid/tumoroid-immune cell co-cultures and oxygen. In some embodiments, the candidate agent is placed on top of the PDT comprising organoid/tumoroid-immune cell co-cultures and oxygen. In some embodiments, the candidate agent is mixed in with PDT comprising organoid/tumoroid-immune cell co-cultures and oxygen. In some embodiments, the candidate agent is mixed in with the comprising organoid/tumoroid-immune cell co-culture medium. In some embodiments, the candidate agent is provided to explants of the PDT.

The continued growth of the PDT comprising organoid/tumoroid-immune cell co-cultures and oxygen comprising media (with or without candidate cells and/or candidate agent) are confirmed by any convenient method, e.g., phase contrast microscopy, stereomicroscopy, histology, immunohistochemistry, electron microscopy, etc. In some instances, cellular ultrastructure and multi-lineage differentiation are assessed. Ultrastructure of the intestinal explants in culture is determined by performing Hematoxylin-eosin staining, PCNA staining, electron microscopy, and the like using methods known in the art.

In some embodiments, the tumoroid/organoid cell culture media and/or the tumoroid/organoid-immune cell culture media can also be used when culturing the tumoroid/organoid and/or tumoroid/organoid immune cell construct with the candidate agent(s).

### Analysis of Tumoroids

In some embodiments the methods of the disclosure further comprise one or more steps of tumoroid analysis. The analysis of the tumoroids and/or organoids may comprise whole-genome sequencing, mRNA sequencing, peptidome profiling and/or microscopy. In some embodiments, tumoroid analysis is used to ensure that the tumoroids and/or organoids are uniform and/or meet expectation, in a form of information discovery and/or information verification. For example, tumoroid analysis is used to determine mRNA transcription differences between organoids and tumoroids, and whether these differences in mRNA transcription are mirrored in differences in protein expression. The presence of specific antigens on organoids/tumoroids are confirmed, and whether any new antigens develop on tumoroids only. The up-regulation of immuno-inhibitory factors in the tumor microenvironment by tumor cells are investigated.

In some embodiments, the immune cells in the tumoroid-immune cell co-culture are subjected to one or more steps of analysis. For example, the analysis of the immune cells may comprise immunophenotyping and/or T-cell receptor sequencing. In some embodiments, analysis is used to check that CAR-T cells express the necessary receptor to recognize tumor cells. Up-regulation of specific receptors recognizing the tumor are investigated. In a particular embodiment, the methods of the disclosure comprise a step of determining HLA-type of the cells, organoids or tumoroids.

In some embodiments, the tumoroid-immune cell co-cultures are subject to one or more analysis steps. In some embodiments, the tumoroid-immune cell co-culture and/or organoid/tumoroid-immune cell co-culture undergoes imaging analysis, flow cytometric analysis, and/or cytokine secretion analysis. This analysis is used to ensure that the tumoroid-immune cell co-cultures are uniform and/or meet expectation.

### Tumoroid Compositions

Disclosed are organoid-immune cell co-cultures useful for investigations relating to diseases, such as cancer and immune diseases, including the identification of suitable treatments for such diseases. This involves in some embodiments preparing co-cultures of organoids and immune cells, particularly disease organoids (such as tumoroids) and immune cells wherein the construct of cells is supplied with oxygen from a media including oxygen, which is exposed to candidate agents for treating diseases and detecting any changes for identifying suitable candidate agents.

Disclosed are human clinical tumor biopsies propagated as PDT-immune cell co-cultures characterized by the culture of the tumor together with their native stromal and immune cells. Using a single 3-dimensional liquid-liquid interface methodology, human PDT-immune cell co-cultures are formed. In a liquid-liquid interface methodology, blood substitute is a pure synthetic compound that is twice as dense as water. In some embodiments, the liquid-liquid interface methodology, blood substitute is a pure synthetic compound that is 1×, 2×, 3×, 4×, 5×, 6×, 7×, 8×, 9× or 10× as dense as water. In each culture well, the liquid-liquid is formed spontaneously as the culture medium floats atop the dense blood substitute. In a liquid-liquid interface the oxygen interacts with the liquid media directly as it is captured by a synthetic hemoglobin and dissolved oxygen is taken up by the liquid media which in a closed system feeds media with dissolved oxygen to the tumoroid during growth. In some embodiments, the liquid-liquid interface is above the tumoroid, below the tumoroid or bisects the tumoroid. In some embodiments, the liquid-liquid interfaces the tumoroid bisects the tumoroid with 10%, 20%, 30%, 40%, 50%, 60%, 70%, 80%, or 90% of the tumoroid under the liquid-liquid interface. In some embodiments, the liquid-liquid interfaces the tumoroid bisects the tumoroid with 10%, 20%, 30%, 40%, 50%, 60%, 70%, 80%, or 90% of the tumoroid above the liquid-liquid interface. These PDT-immune cell co-cultures accurately recapitulated clinical pathology upon histologic analysis. In some embodiments, the PDT-immune cell co-culture comprise preserved endogenous immune subsets including CD3+, B cells (CD20), Macrophages (CD68), NK cells (CD16), or a combination thereof. In some embodiments, the PDT-immune cell co-culture further comprise tumor-infiltrating lymphocytes (TILs), CD8+, PD1+, T helper, and NK cells amongst the tumor cells. In some embodiments, the PDT-immune cell co-cultures described herein comprise immunotherapeutic PD-1 antibody nivolumab induced T-cell activation and cytolytic activity, indicating successful in vitro recapitulation of immune checkpoint blockade and anti-tumor immunity. These tumor PDT-immune cell co-culture models facilitate the in vitro investigation of human tumor immunity with application to modeling drug candidate screening for development of new treatments and patient immunotherapy responses in a clinical setting.

In some embodiments, the PDT-immune cell co-cultures are derived from primary and/or metastatic tumors. In some embodiments, the PDT-immune cell co-cultures are derived from tissue from surgical resection procedures by plating mechanically dissociated tumor fragments in an oxygen containing media using a liquid-liquid interface culture system. In some embodiments, the tumor fragments are expanded to form tumoroids, which are cultured for extended periods and expanded through passage and secondary plating. In some embodiments, the PDTs display striking recapitulation of histological architecture of the tumors from which they were derived. In some embodiments, the PDTs showed cribriform growth, desmoplasia, and intraluminal necrosis. In some embodiments, the PDTs preserved stroma as characterized by vimentin+ cells. In some embodiments, the PDT-immune cell co-cultures are cryopreserved. In some embodiments, the PDT immune cell co-cultures are cryopreserved. In some embodiments, the PDT-immune cell co-cultures are cryopreserved with or without media. In some embodiments, the PDT-immune cell co-cultures are cryopreserved with or without PBMC. In some embodiments, the PDT-immune cell co-cultures are cryopreserved for 1 hour to 10 years. In some embodiments, the PDT-immune cell co-cultures are cryopreserved without the loss of stromal cells or architecture. In some embodiments, PDTs are re-programed using gene editing techniques. In some embodiments, PDT-immune cell co-cultures are re-programed using gene editing techniques. In some embodiments, PDTs are re-programed using Cas-9 techniques. In some embodiments, re-programed PDTs are grafted into the patient from which they were derived. In some embodiments, PDTs are xenografted into immunocompromised mice to generate transplantable models of cancer progression, and then re-derived as organoid cultures thereafter.

The liquid-liquid interface PDT culture method is cell type agnostic, patient agnostic, tissue site agnostic, and disease agnostic. In some embodiments, tissue used to form a PDT is collected from colon, pancreas, and lung, as well as rarer subtypes such as bile duct and endometrium for which cell lines are not readily available. In some embodiments, the PDTs are generated from a wide range of tumor stages and grades including, but not limited to, stage 1, stage 2, stage 3, and stage 4 cancers. In some embodiments, the PDTs are analyzed by sequencing, Western blotting, ELISA-based detection, Northern blotting, real time PCR, and/or RT PCR.

Also provided is a tumoroid-immune cell co-culture obtainable or obtained by the methods of the disclosure. Also provided is a population of tumoroids obtainable or obtained by methods of the disclosure, i.e., co-cultured with immune cells. Also provided is a tumoroid-immune cell co-culture medium suitable for use in methods of the disclosure. Also provided is a tumoroid or organoid in a medium comprising, consisting of, or consisting essentially of oxygen. Also disclosed are kits comprising, consisting of, or consisting essentially of a tumoroid, or tumoroid-immune cell co-culture of the disclosure.

Disclosed are tumor microenvironment (TME). TMEs are able to accelerate the drug development process by building tumoroids (tumor organoids) on a platform COMPASS (Custom Organoid Model Platform for Accurate and Speedy Solutions). In some embodiments, the tumoroid is derived from live or cryopreserved tumor chunks, mechanically minced, and separated into smaller pieces that are allowed to recapitulate the original tumor architecture by growing the TME intact pieces on Matrigel-based matrix using serum free media. In some embodiments, PDTs mimic the structural and functional integrity of the original source tumor due to the retention of epithelial and stromal cells. In some embodiments, PDTs are built, characterized using RNA seq, and tested across five generations for genetic drift. In some embodiments, the PDTs are bio-banked for rapid-deployment and reproducibility. In some embodiments, the PDTs are allowed to grow to 50-500 mM³ in volume with inbuilt tumor size variations prior to being used for modality agnostic testing to get efficacy, biomarker, combination studies data and other readouts. In some embodiments, immuno-competence is built into the PDTs by adding allogenic PBMCs during the growth process. In some embodiments, immuno-competence is built into the PDTs by adding allogenic PBMCs to the tumor chunk, to the formed tumoroid or both. In some embodiments, the PDTs are used to test a range of immune-based therapeutics. In some embodiments, the tumor immune-competent tumoroids grow in media comprising physiological levels of oxygen. In some embodiments, the tumor immune-competent tumoroids grow in media comprising physiological levels of oxygen and using synthetic hemoglobin. In some embodiments, the tumor immune-competent tumoroids grow in media comprising physiological levels of oxygen, using synthetic hemoglobin and a perfusion system for testing pH indexed therapeutic response data.

### Compass

COMPASS is a TME based platform for a range of molecules, biologics, antibodies, combination therapies and more. In some embodiments, COMPASS is an immunocompetent platform conducive for testing a range of immune targeting therapeutics and/or a dynamic platform with physiological flow of oxygen and/or carbon dioxide allowing longitudinal studies with or without immune components.

The COMPASS models provide modality-agnostic models that provide effective prediction of clinical outcomes during preclinical testing of drug candidates. In some embodiments, the COMPASS model is an immunotherapeutic modality-receptive model, in order to effectively evaluate the capacity of immunotherapies in generating adaptive anti-tumor responses.

In some embodiments, a COMPASS model platform provides translational qualification of the desired physiology and functionality that elicits the expected responses compared to standard or reference modalities.

In some embodiments, the three-dimensional COMPASS platform is designed to provide predictive human efficacy data to enable the recapitulation of patient-specific intra-tumoral interactions to facilitate drug development and/or patient treatment.

Custom Organoid Modelling Platform for Accurate & Speedy Solutions (COMPASS) is a functional assay to assess PDT-immune-cell response to candidate agents within a clinically actionable (14-42 day) time frame. In some embodiments, the clinically actionable time frame is 1-7 days, 7-14 days, 7-21 days, 7-28 days, 7-35 days, 7-42 days, 7-49 days, 7-56 days, 7-63 days, 7-70 days. 7-77 days or 7-85 days. In some embodiments, the clinically actionable time frame is 3-7 days, 3-14 days, 3-21 days, 3-28 days, 3-35 days, 3-42 days, 3-49 days, 3-56 days, 3-63 days, 3-70 days. 3-77 days or 3-85 days. In some embodiments, the clinically actionable time frame is 1-14 days, 14-21 days, 14-28 days, 14-35 days, 14-42 days, 14-49 days, 14-56 days, 14-63 days, 14-70 days. 14-77 days or 14-85 days. In some embodiments, PDT-immune-cell co-cultures are established from a variety of distinct surgically-resected tumor biopsies representing more than one cancer type.

In some embodiments, the COMPASS system is a modality and cancer indication agnostic platform. In some embodiments, the COMPASS system enables researchers to obtain efficacy and biomarker data on any modality of interest.

Disclosed is a COMPASS system comprising, consisting of, or consisting essentially of PDT-immune-cells. Disclosed is a COMPASS system comprising, consisting of, or consisting essentially of PDT-immune-cells on a non-scaffold-based system. Disclosed is a COMPASS system comprising, consisting of, or consisting essentially of PDT-immune-cells on a natural structural-biochemical matrix.

Disclosed is a COMPASS system comprising, consisting of, or consisting essentially of PDT-immune-cells and at least one PBMCs.

Disclosed is a COMPASS system comprising, consisting of, or consisting essentially of PDT-immune-cells on a scaffold and apical oxygenation only. Disclosed is a COMPASS system comprising, consisting of, or consisting essentially of PDT-immune-cells comprising PBMCs, on a scaffold and apical oxygenation only.

Disclosed is a COMPASS system comprising, consisting of, or consisting essentially of PDT-immune-cells on a scaffold, with basal oxygenation and peri-cellular perfusion. Disclosed is a COMPASS system comprising, consisting of, or consisting essentially of PDT-immune-cells comprising PBMCs, on a scaffold and basal oxygenation and peri-cellular perfusion.

In some embodiments, the COMPASS system is TME centric, modality agnostic, assesses efficacy, assesses biomarkers, has physiological oxygen flow, is capable of performing longitudinal studies, is capable of performing combination studies and combinations thereof. In some embodiments, the longitudinal studies are studies that last days, weeks, months, or years. In some embodiments, the longitudinal studies are studies that last 0-21 or 0-42 days. In some embodiments, the longitudinal studies are studies that last 0-5, 0-10, 0-20, 0-30, 0-40, 0-50, 0-60, 0-70, 0-80, 0-90, or 0-100 days. In some embodiments, the system can produce a result/readout in about 4 months compared to 12-24 months with other methods. In some embodiments, the system does not include a process (i.e., produces a readout) that lasts longer than 3, 4, 5, 6, 7, 8, 9, 10, 11, 12, 13, 14, 15, 16, 17, 18, 19, 20, 21, 22, 23 or 24 months. In some embodiments, the system produces a result/readout in less than 1, 2, 3, 4, 5, 6, 7, 8, or 9 months.

In some embodiments, the rate of growing PDT-immune cell from tissue is 80-85% compared to <20% for organoids or tumoroids. In some embodiments, tests done on the PDT-immune cells are applicable to humans 80% of the time compared to <55% with others. In some embodiments, the data from PDT-immune cells is submitted for IND filing.

In some embodiments, the platform and PDT-immune cells are used for assessing drug efficacy, combination therapies, drug repositioning and combinations thereof. In some embodiments, the platform and PDT-immune cells are used to produce improved therapeutics, additive/synergistic data re drug combinations, new indications, biomarker discovery and combinations thereof.

In some embodiments, the system/method/compositions disclosed herein enables the testing of a drug, a small molecule, large molecule, or combinations thereof in combination with standard of care therapies. In some embodiments, the system/method/compositions disclosed herein enables the identification of biomarkers that respond to a therapy. In some embodiments, the system/method/compositions disclosed herein provides mechanistic insights into the drug and/or drug combination. In some embodiments, the system/method/compositions disclosed herein enables identification of a subset common to both responders that were identified to be utilized for developing companion diagnostic biomarkers of response and or patient stratification. In some embodiments, the system/method/compositions disclosed herein helps define the role of the genes that change significantly post treatment and help identify pathways of response or resistance. In some embodiments, the data produces by the system is submitted as part of an IND filing package.

In some embodiments, the system/method/compositions disclosed herein enables the evaluation of Drug X (a candidate agent). In some embodiments, the system/method/compositions disclosed herein show that Drug X increased TIL incorporation over time. In some embodiments, the system/method/compositions disclosed herein data show Drug X in combination with anti-PD1 could drive antigen presentation in PDTs and augment cell killing. In some embodiments, the system/method/compositions disclosed herein show that the effect of Drug X and anti-PD1 inhibitor was additive but not synergistic. In some embodiments, the system/method/compositions disclosed herein show that the effect of Drug X and anti-PD1 inhibitor is synergistic. In some embodiments, the platform produces a result/readout to take Drug X to Phase 2 trials.

In some embodiments, system/method/compositions disclosed herein shows potential efficacy and resistance insights against established drug regimens. In some embodiments, the system/method/compositions disclosed herein provides initial comparative insights for tumor-immune-drug interplays. In some embodiments, the system/method/compositions disclosed herein reveals efficacies and potential conditions for optimal drug efficacy. In some embodiments, the system/method/compositions disclosed herein allows for the re-assessment of previously drugs which have clinical failure. In some embodiments, the system/method/compositions disclosed herein allows for the re-assessment previously clinically successful drugs. In some embodiments, the system/method/compositions disclosed herein allows for the re-assessment of failed pre-clinical drugs. In some embodiments, the system/method/compositions disclosed herein allows for the re-assessment of clinically successful pre-clinical drugs.

In some embodiments, Drug X is an immune enhancer as determined by the system/method/compositions disclosed herein. In some embodiments, Drug X can activate cold tumors as determined by the system/method/compositions disclosed herein. In some embodiments, Drug X can turn a cold tumor hot via increased TIL incorporation as determined by the system/method/compositions disclosed herein.

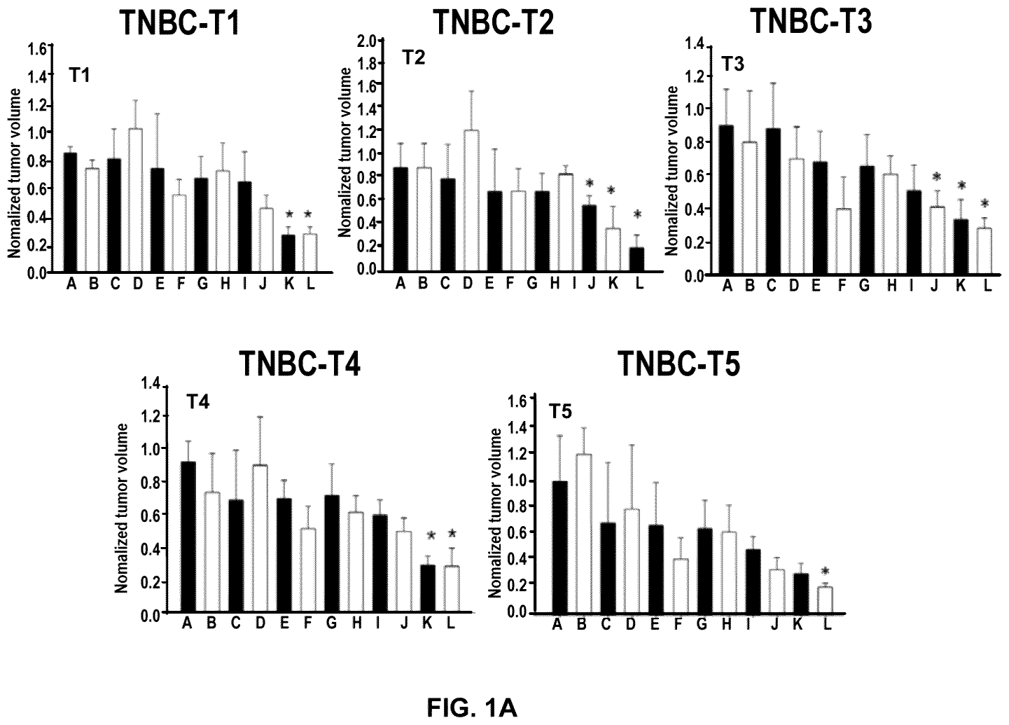

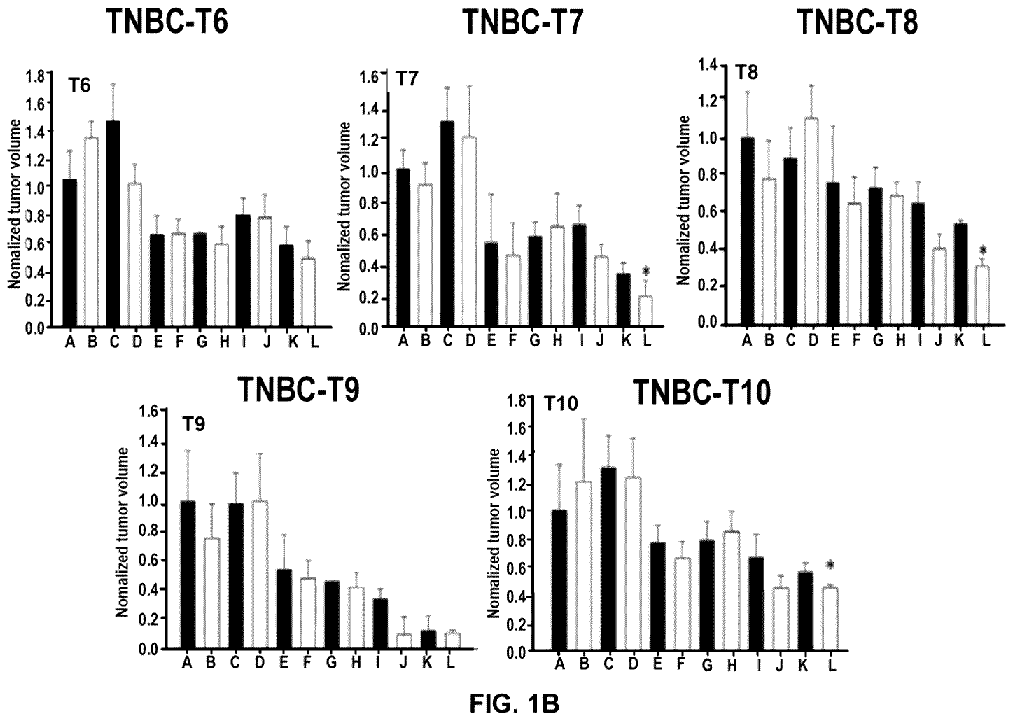

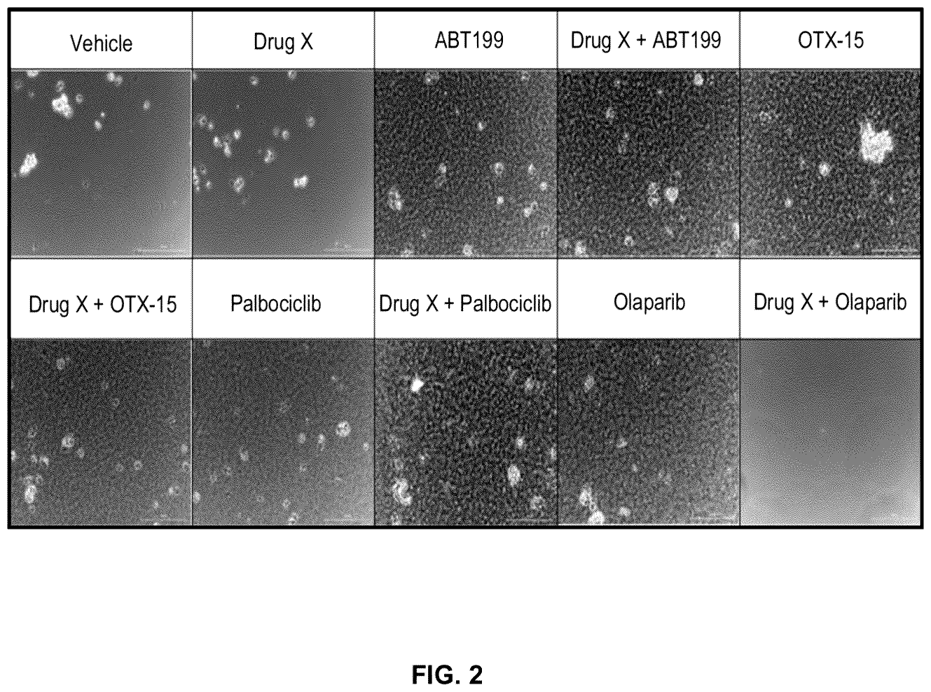

In some embodiments, the PDT-immune cell co-cultures received different combination of Olaparib and Drug X at different concentrations against 10 Triple Negative Breast Cancer (TNBC) COMPASS1 tumoroids (FIGS. 1A and 1B). After 14 days of treatment with Drug X and a variety of combinations of Drug X with other drugs, cell images of tumor volume using COMPASS 1 platform were taken (FIGS. 2-4). For more detail see Example 1.

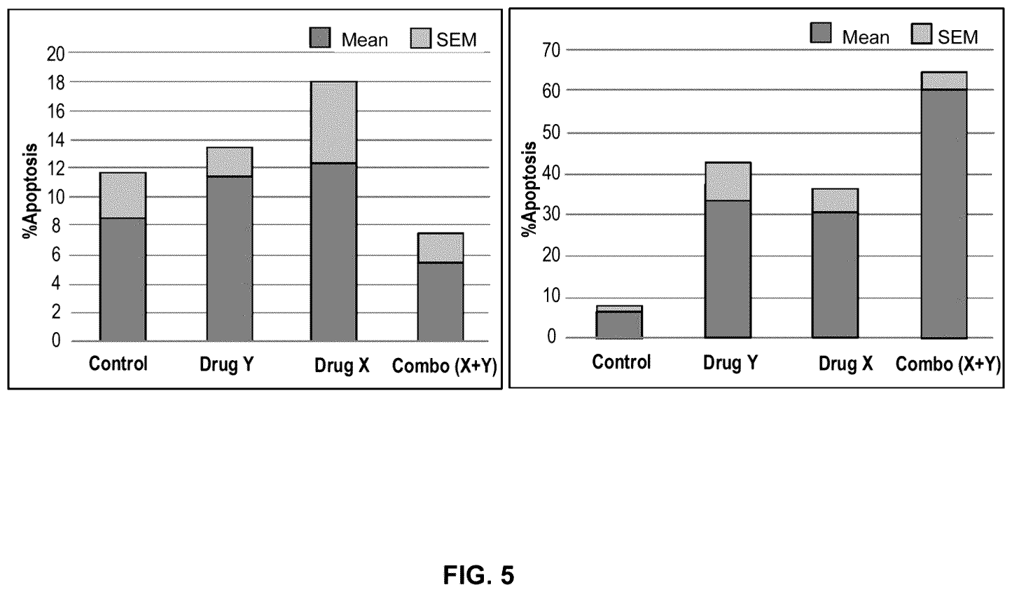

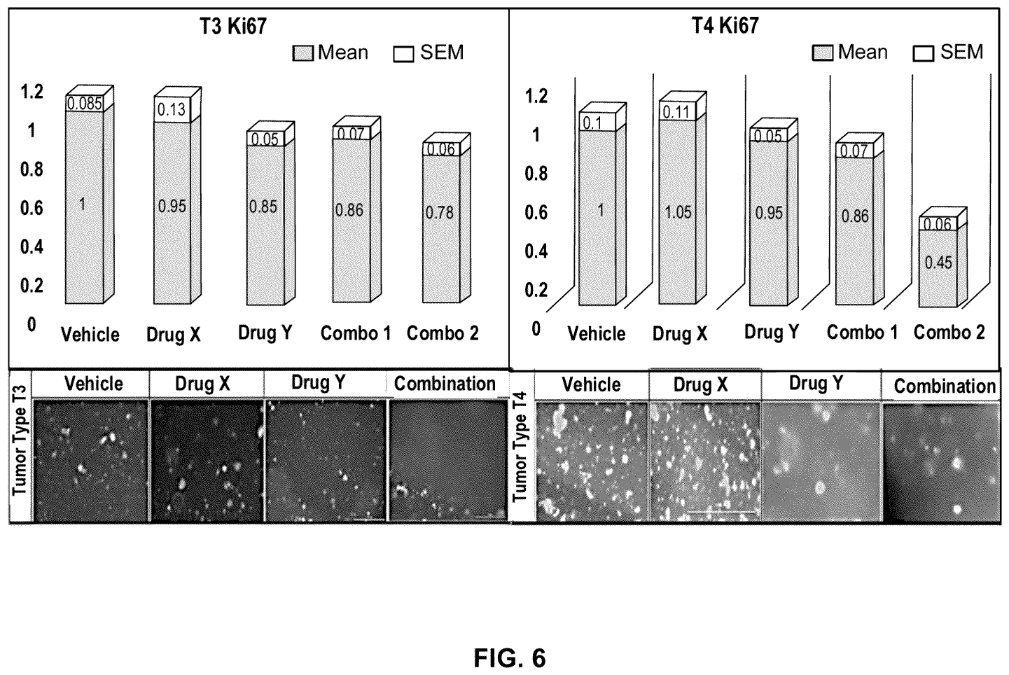

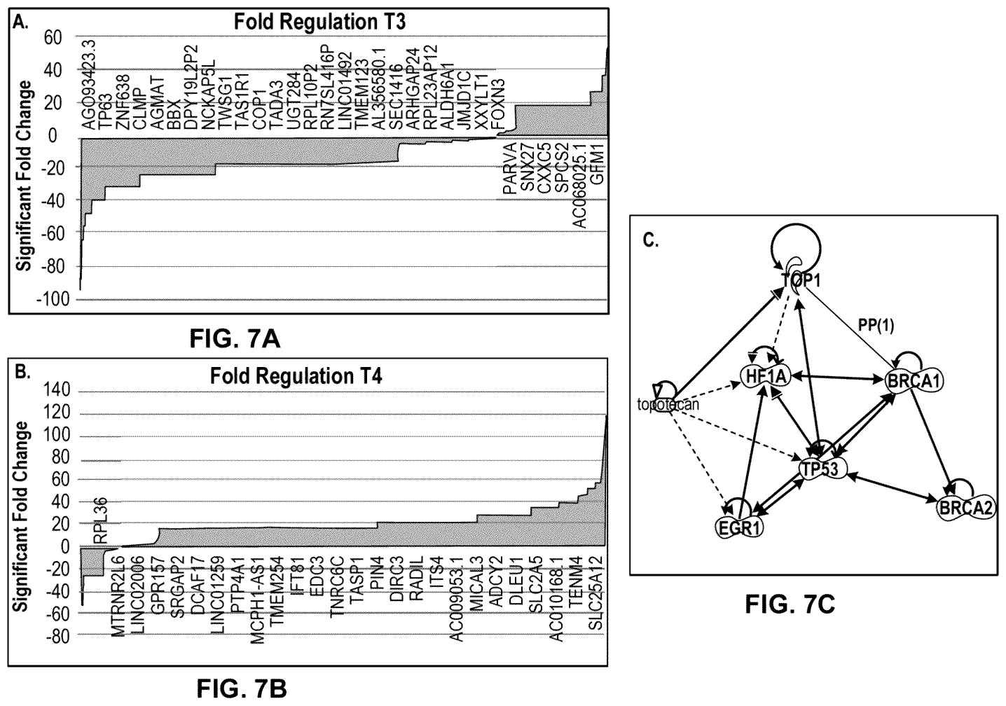

In some embodiments, PDT-immune cell co-cultures received different combination of Drug X, Y, and combinations thereof against TNBC COMPASS1 tumoroids. The Mechanism of cell death (FIG. 5), cell proliferation (FIG. 6), and RNA sequence analysis (FIGS. 7A-C). Effect of drug treatment was observed by comparing the tumor volume changes using imaging analysis of 3 biological replicates. The combination of efficacy and biomarker provided mechanistic insights into the drug combination. Downstream biomarker analysis using RNA seq, followed by pathway analysis, was done. This helped identification of biomarkers of response to and escape from therapy. For more detail see Example 2.

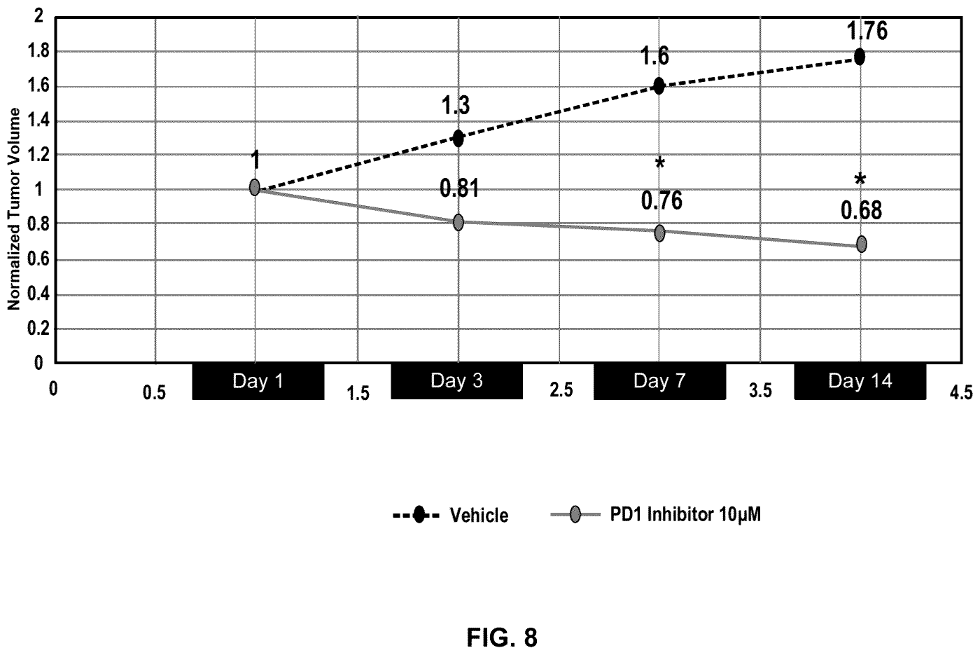

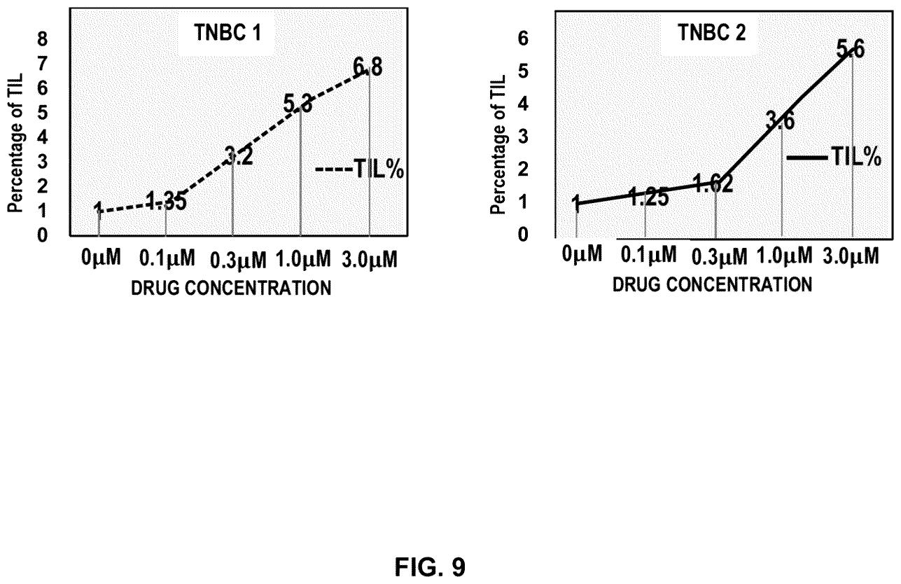

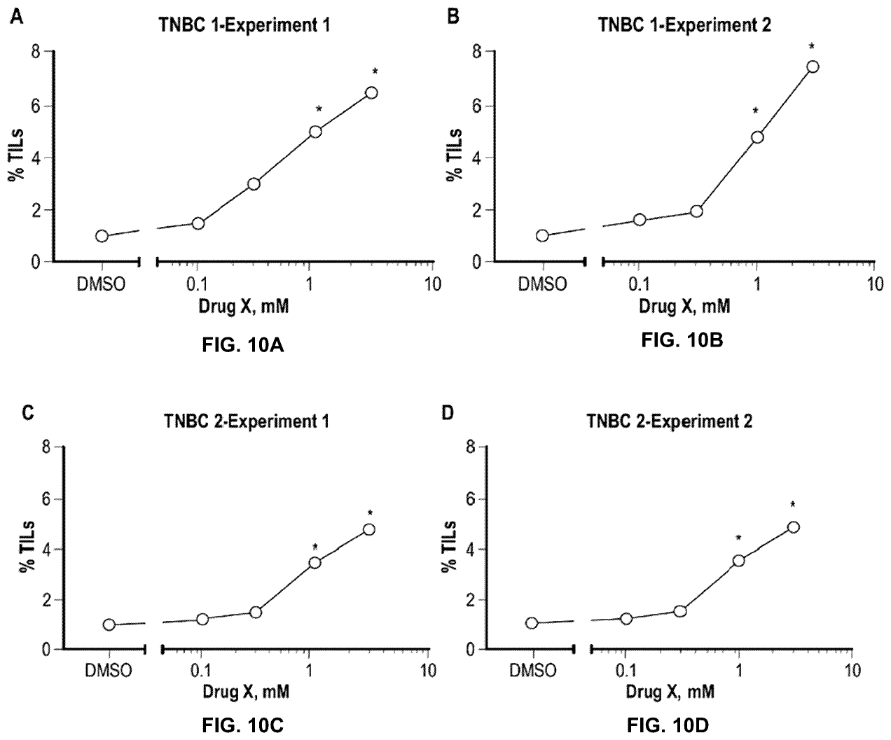

In some embodiments, PDT-immune cell co-cultures received PBMCs (COMPASS 2 platform). PDT cultured with PBMC show an immune checkpoint mediated reduction in tumor volume (FIG. 8) and increase in percentage of TILs (FIG. 9), and increased TIL infiltration into the PDT (FIG. 10) in the COMPASS 2 platform. PDL-1-positive TNBC PDTs in COMPASS 2 model (in presence of PBMCs) demonstrated statically significant response to treatment with an anti-PD1 inhibitor. This result was not observed on the same model unaugmented with PBMCs. Sequential addition of drug X, which increased TIL infiltration, followed by anti-PD1 therapy showed a significant tumor volume reduction-demonstrating the utility of these models in testing combination therapies with checkpoint inhibitors. For more detail see Example 3.

In some embodiments, using the COMPASS 1 tumoroid platform, biomarkers can be identified that respond-to and escape-from therapy when treated—providing mechanistic insights into the drug combination. In some embodiments, using the COMPASS 2, the effect of the combination of efficacy and biomarker studies, involving immunophenotyping, can help develop mechanistic insights on possible changes amongst the interplay of different immune cells and their receptors. In some embodiments, the PDT (using a COMPASS 2 model) demonstrates an immune checkpoint-mediated reduction in tumor volume—an effect not observed on the non-immunocompetent version of the same model, or the expected responses compared to standard or reference modalities. In some embodiments, the oxygenation status (dynamic vs static) (using COMPASS 3) impacts the effect of HSP90i on soft tissue sarcoma. In some embodiments, TIL regulation, CAR-T, NK cells (and their combination therapies) improve antigen presentation and cell killing, ultimately activating cold tumors. In some embodiments, immunotherapy, pH modulation, hypoxia-based therapeutics done in combinations with small molecules, biologics and established immune therapeutic (e.g., anti-PD1 or anti-CTLA4), the role of oxygen and combinations thereof improve analysis of a therapy.

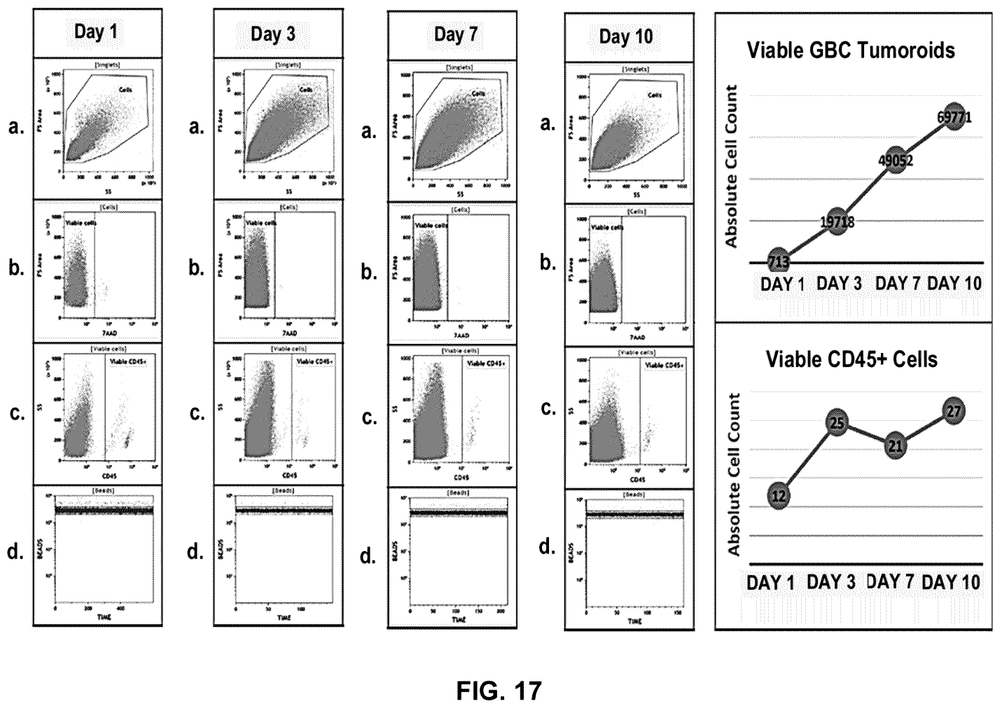

In some embodiments, the PDT comprises immune components. In some embodiments, the immune component is CD3, CD20, CD16 and/or CD68. See FIG. 13 for the presence of immune components in immune competent Carcinosarcoma tumoroids. See FIG. 14 for the presence of immune components in immune competent Gall Bladder Carcinoma tumoroids. In some embodiments, the PDT is assessed for live-dead cells (See FIGS. 17-19). In this study the COMPASS 2 model platform was used to test the immunocompetence of disclosed PDTs and to investigate the infiltration depth, extent, and length of survival for T cells (CD3+, CD8+, CD4+) B cells (CD20), macrophages (CD68, CD68+, CD20+OR CD19+CD16) and NK cells (CD16, CD56+/CD16+) in human uterine carcinosarcoma and GBC. This Immunofluorescence data demonstrated that the COMPASS 2 model platform with intact immune components including T cells, B cells, macrophages, and NK cells has the potential to bridge the gap of standard or reference modalities by recreation of TME features as close as possible to in vivo conditions. Together these results clearly establish the growth conditions of the disclosed PDTs as parameters conducive for growth of tumor, stroma, and immune component in both GBC and Sarcoma Tumoroids. The immune components evaluated in the study showed robust presence on Day 3 and Day 7 post-growth without any replenishment.

Using FACS analysis, the presence of lymphocytes, monocytes, and granulocytes in the COMPASS 2 PDTs were detected. This data demonstrates the presence of T cells (CD3+) and their subsets (CD4+, CD8+) and the viability of both NK cells and B cells up to 7 days in the COMPASS 2 model. Lastly, growth media supported these immune cells in the system from Day 1 through 7 with a reduction by Day 14. In addition to demonstrating the presence and survival of immune cells, it was established that the platform is agnostic to any particular tumor. For more detail see Example 4.

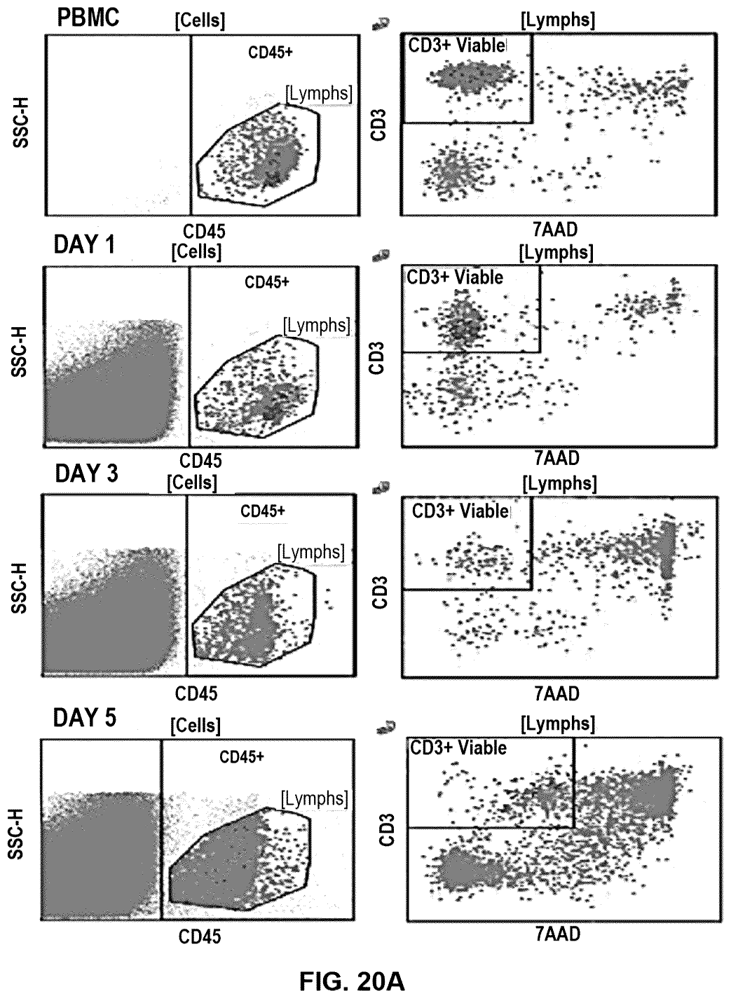

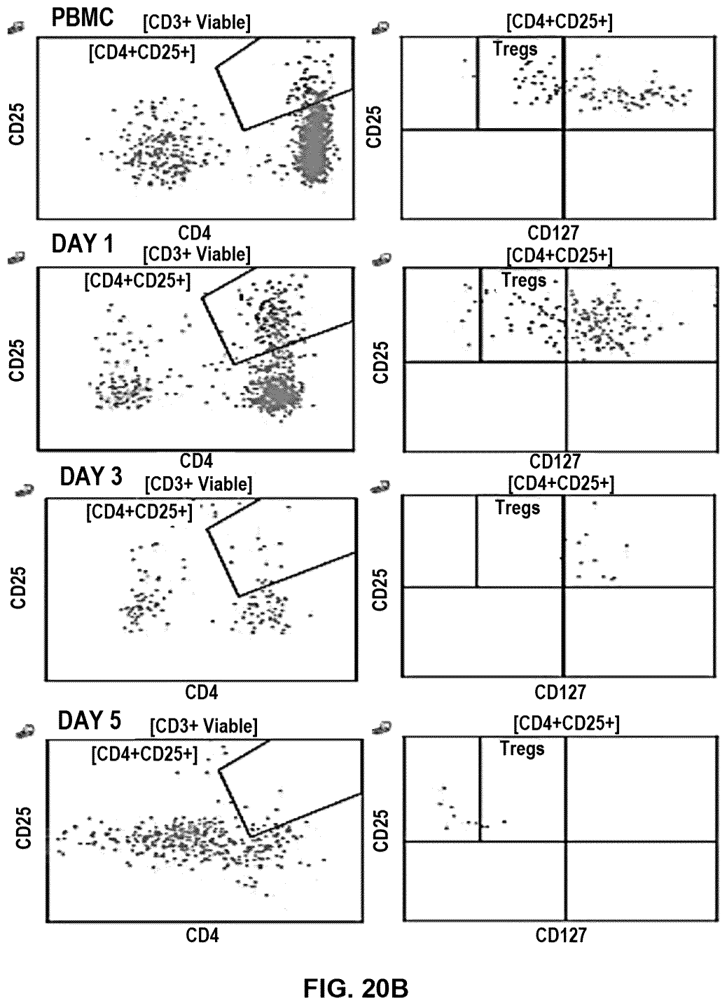

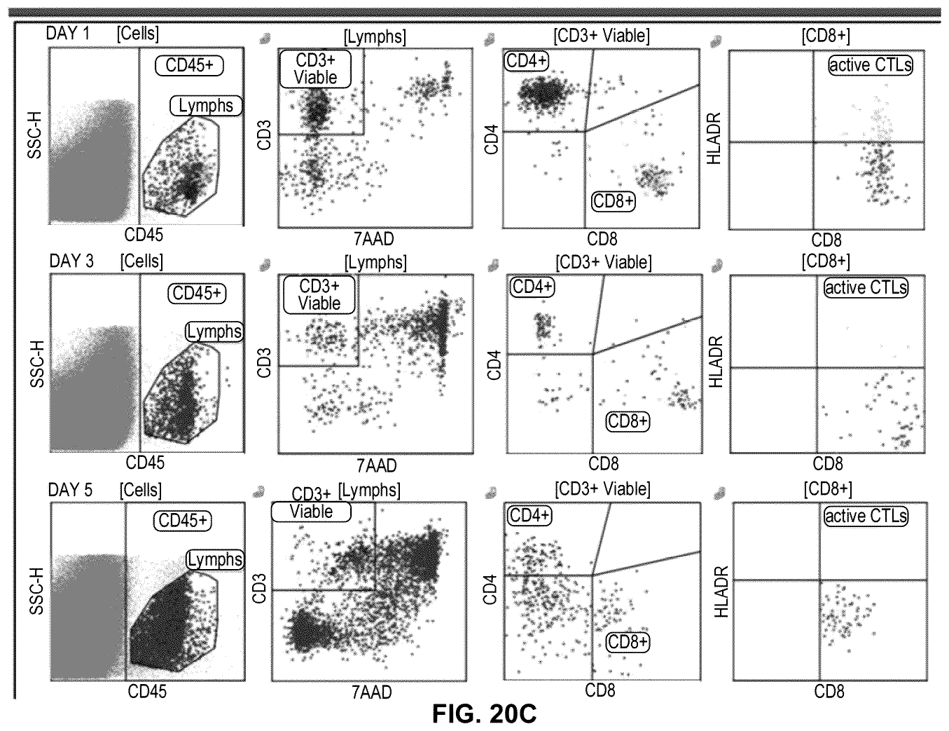

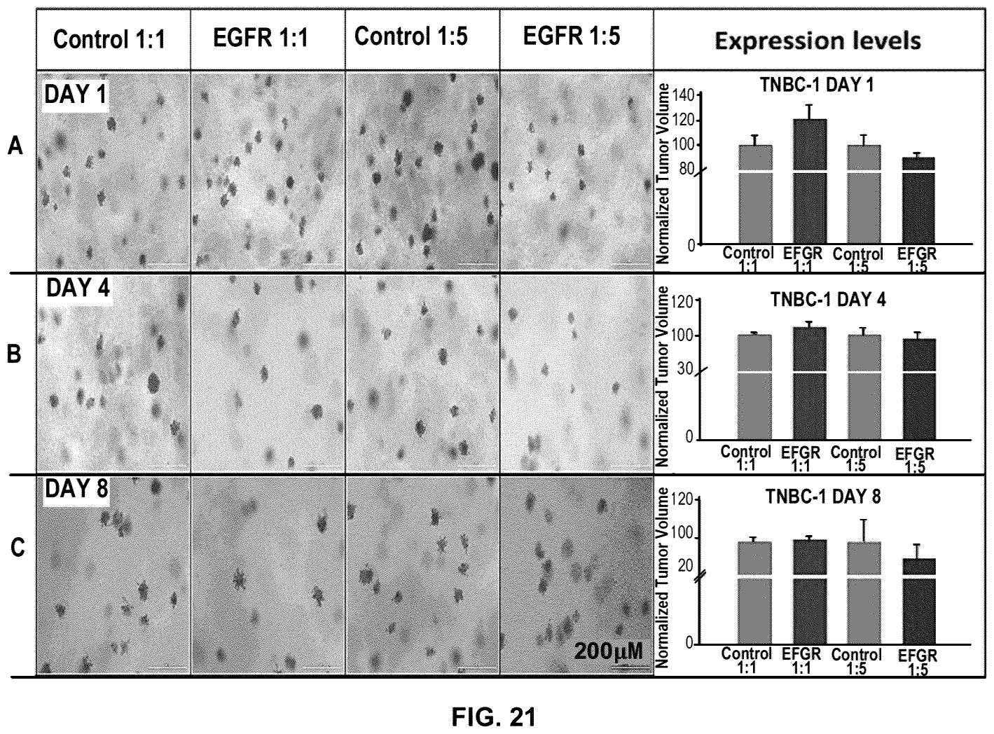

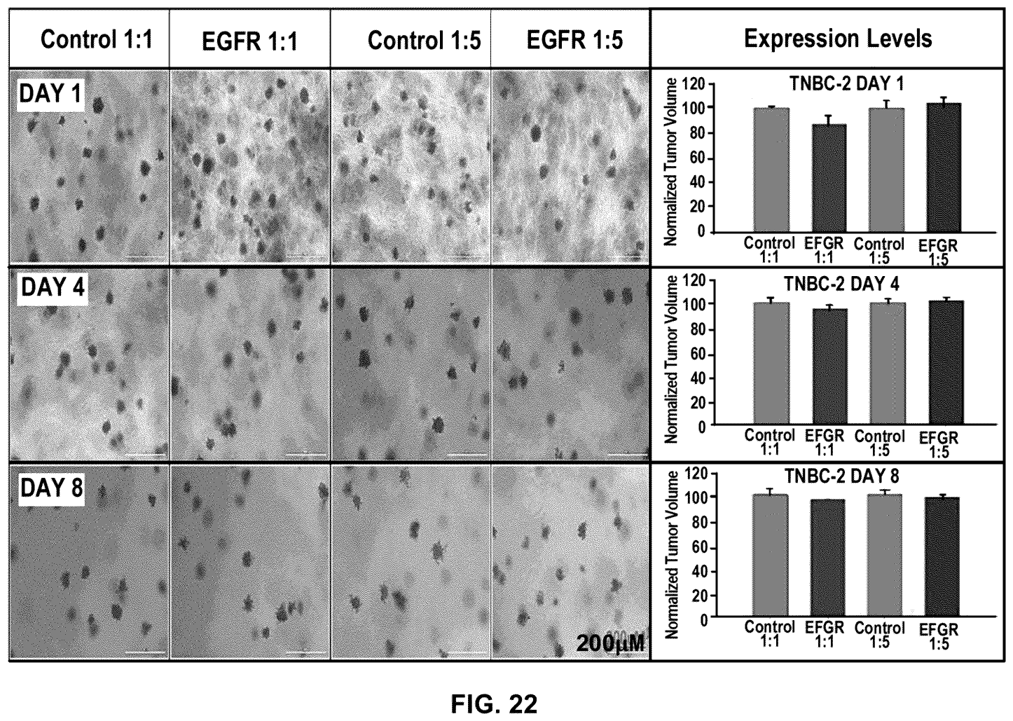

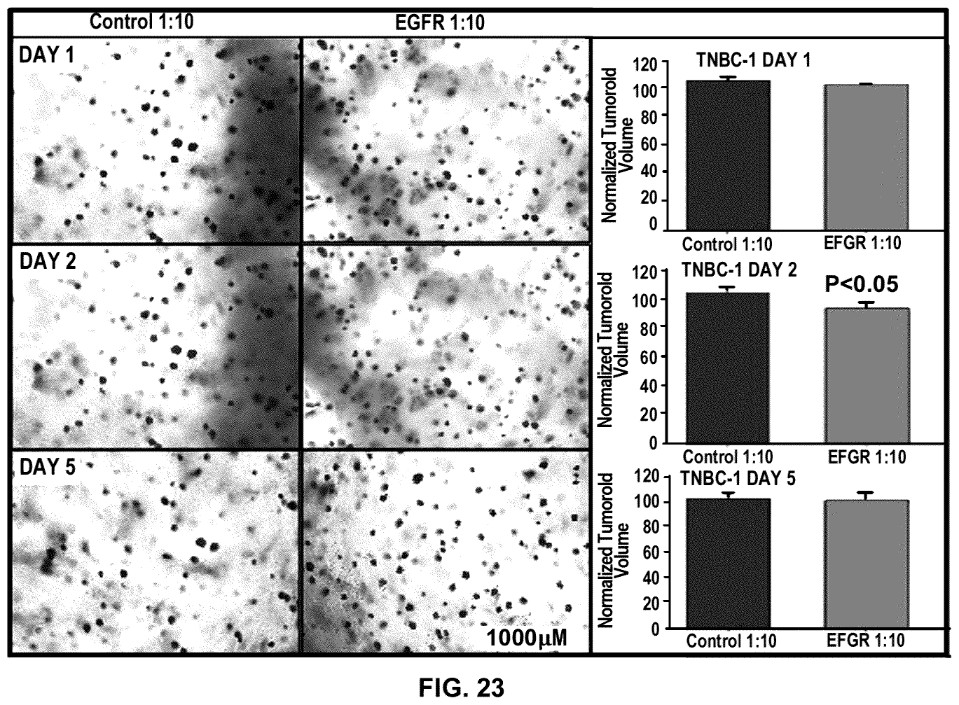

In some embodiments, PDTs comprise Treg cells. In some embodiments, PDTs comprise Treg cells and PBMC (See FIG. 20). In some embodiments, PDTs comprise CTL activation of cells. In some embodiments, PDTs comprise CTL activation of cells and PBMC (See FIG. 21). In some embodiments, PDTs comprise T cells. In some embodiments, PDTs comprise T cells and PBMC (See FIG. 22). In some embodiments, PDTs comprise lymphocyte subsets, such as T cells (CD3+), cytotoxic T lymphocytes (CD8+CD3+), and activated CTLs activation of cells. In some embodiments, PDTs comprise lymphocyte subsets, such as T cells (CD3+), cytotoxic T lymphocytes (CD8+CD3+), and activated CTLs activation of cells and PBMC (See FIG. 23). In some embodiments, PDTs comprise CAR-T cells. In some embodiments, PDTs comprise CAR-T and PBMC (See FIGS. 21-23).

In some embodiments, COMPASS 2/3 is able to mimic TME features closely by supporting tumor, stroma, and immune component growth. In some embodiments, COMPASS 2/3 forms a PDT with developmental and functional stages of CAR-T cells including activation, memory, and/or exhaustion. In some embodiments, COMPASS 2/3 forms a PDT with significant TNBC-1 tumor volume decrease with EGFRvIII-targeted CAR-T cell treatment, and GBC organoid co-cultures with PBMCs showed Treg and CTL expression until Day 3. In some embodiments, COMPASS 2/3 is used for functional testing of CAR-T cells, Tregs, and CTLs. In some embodiments, a COMPASS 2/3 PDT comprises T cell infiltration impacting TME. In some embodiments, the COMPASS 2/3 PDT comprises Tregs and CTLs as potential diagnostic markers for GBC. In some embodiments, the COMPASS 2/3 platform is used for studies that require ex-vivo recapitulation of response to CAR-T therapy, T-cell regulation, infiltration, and/or exhaustion by elucidating the crucial role of immune response in mediating the anti-tumor effects of CAR-T therapy. For more detail see Example 5.

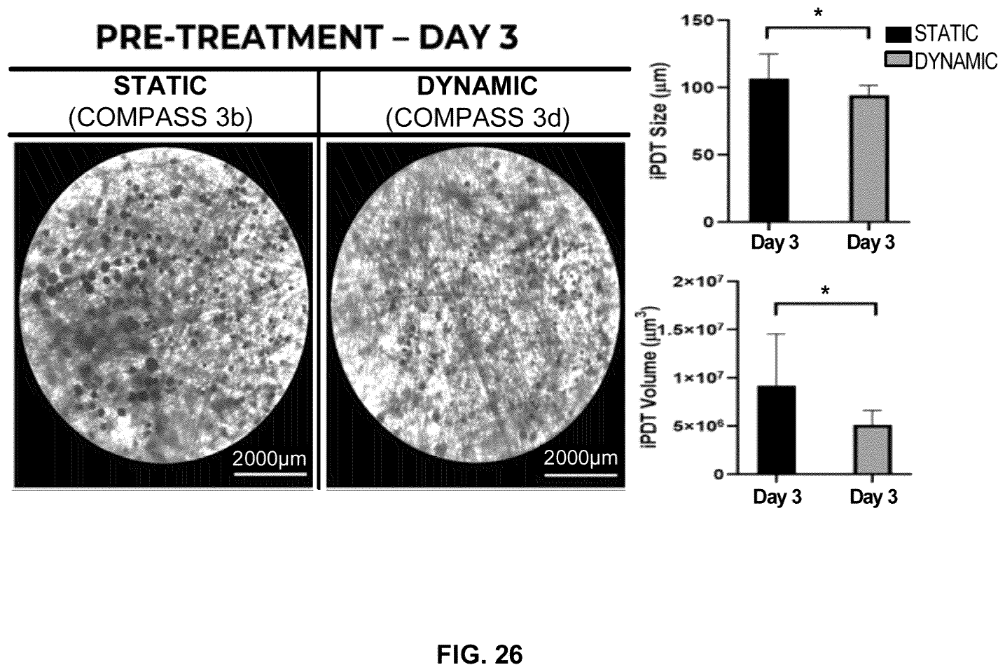

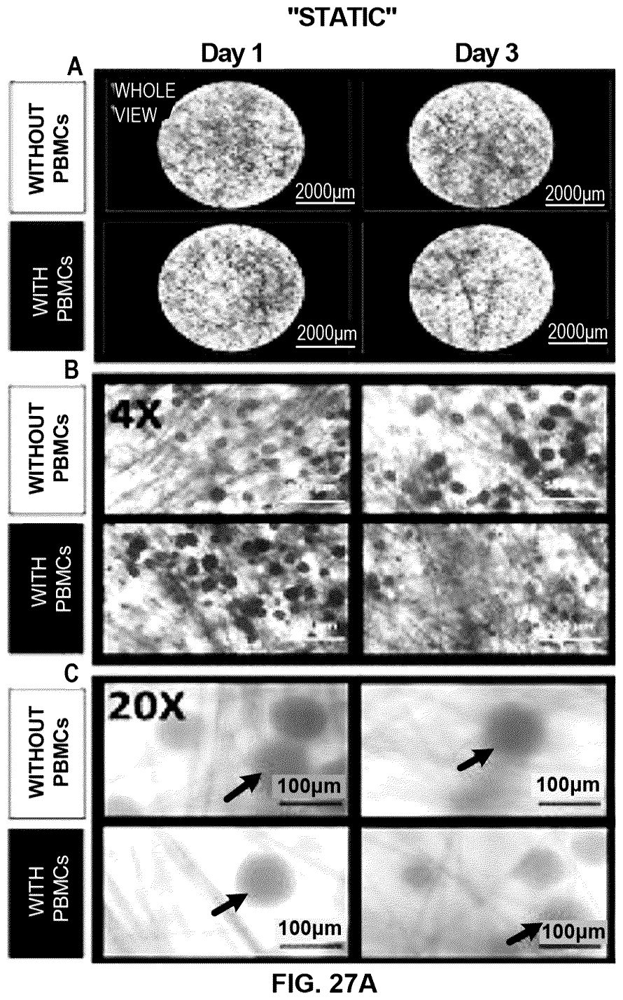

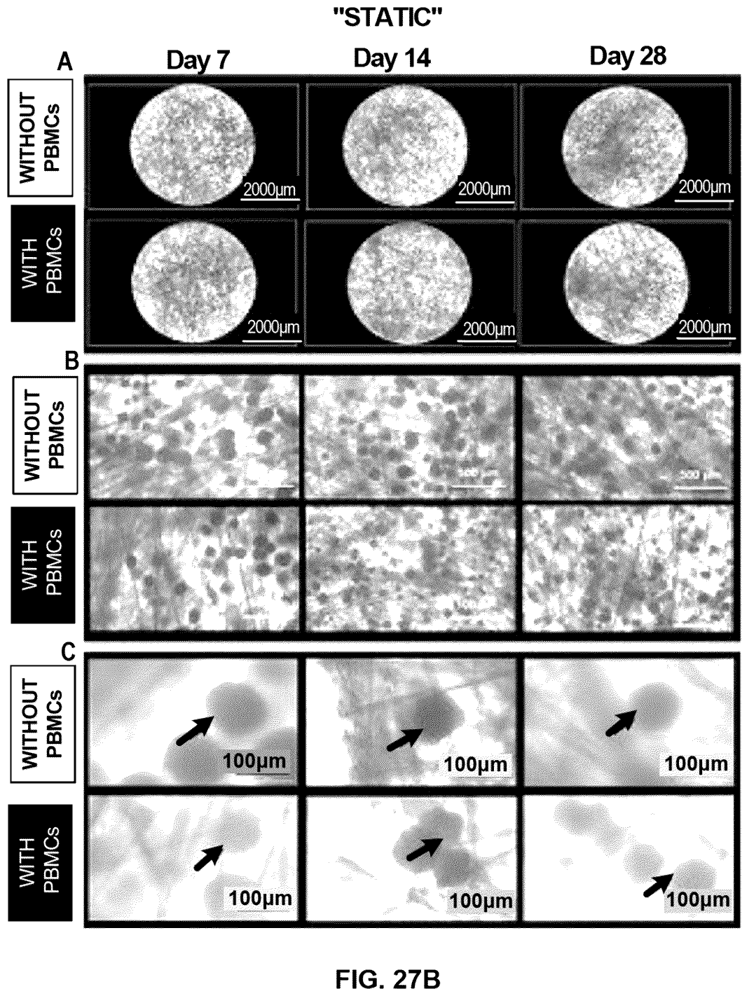

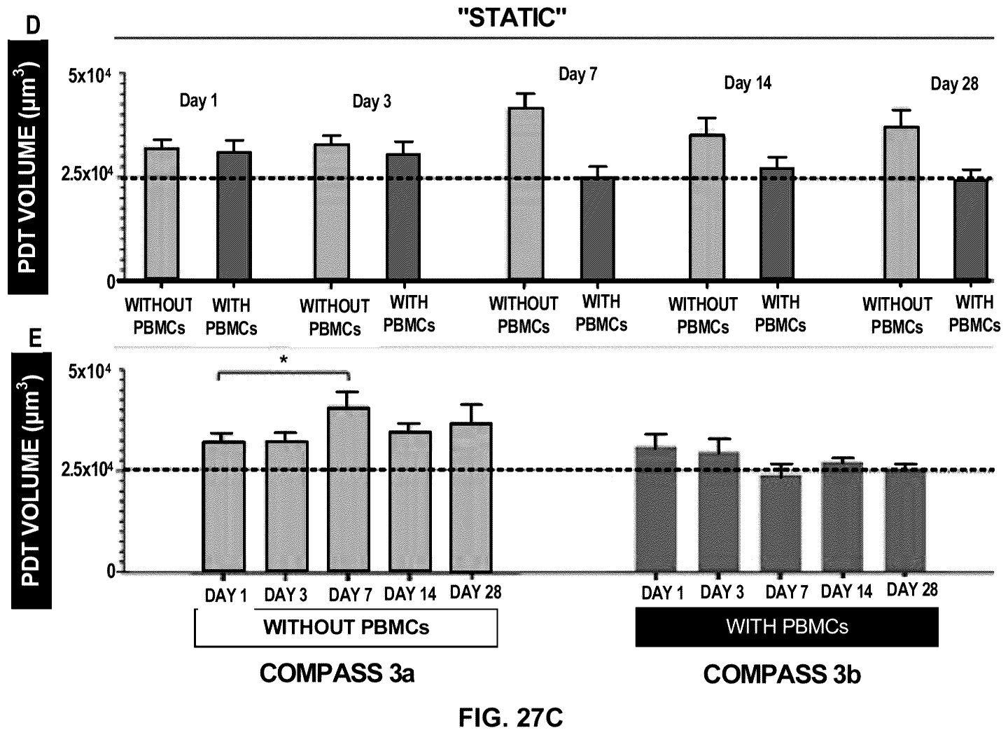

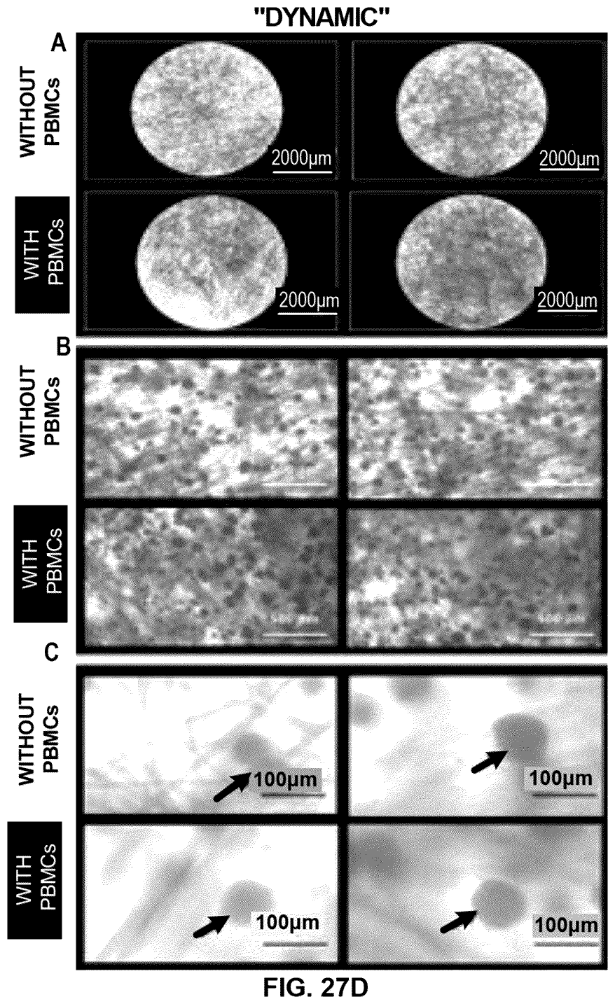

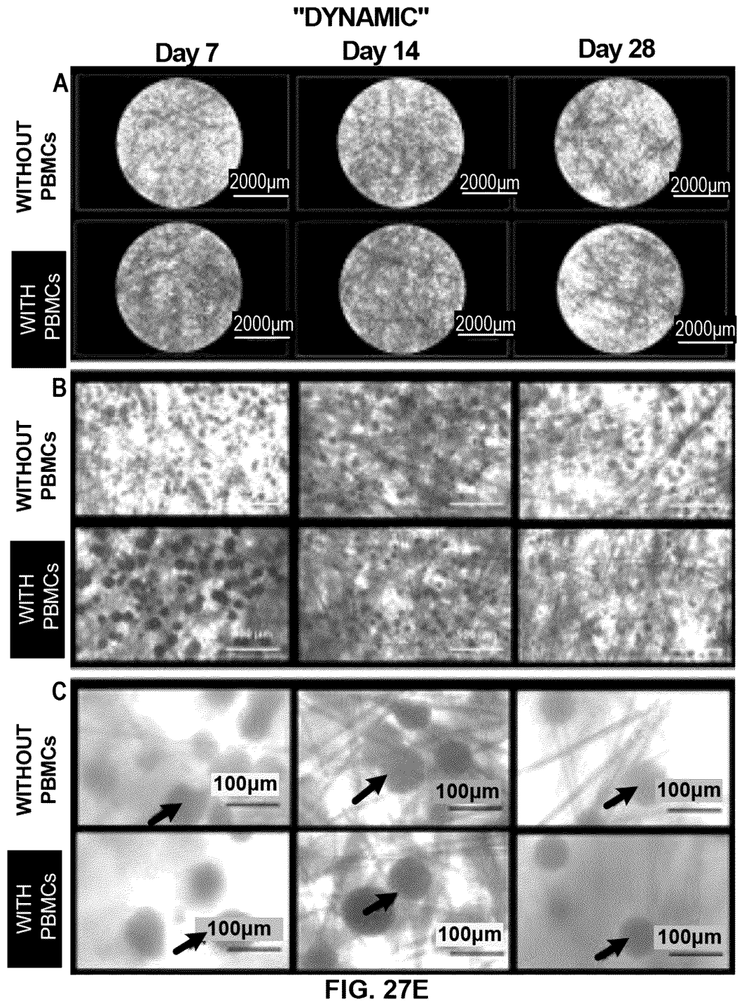

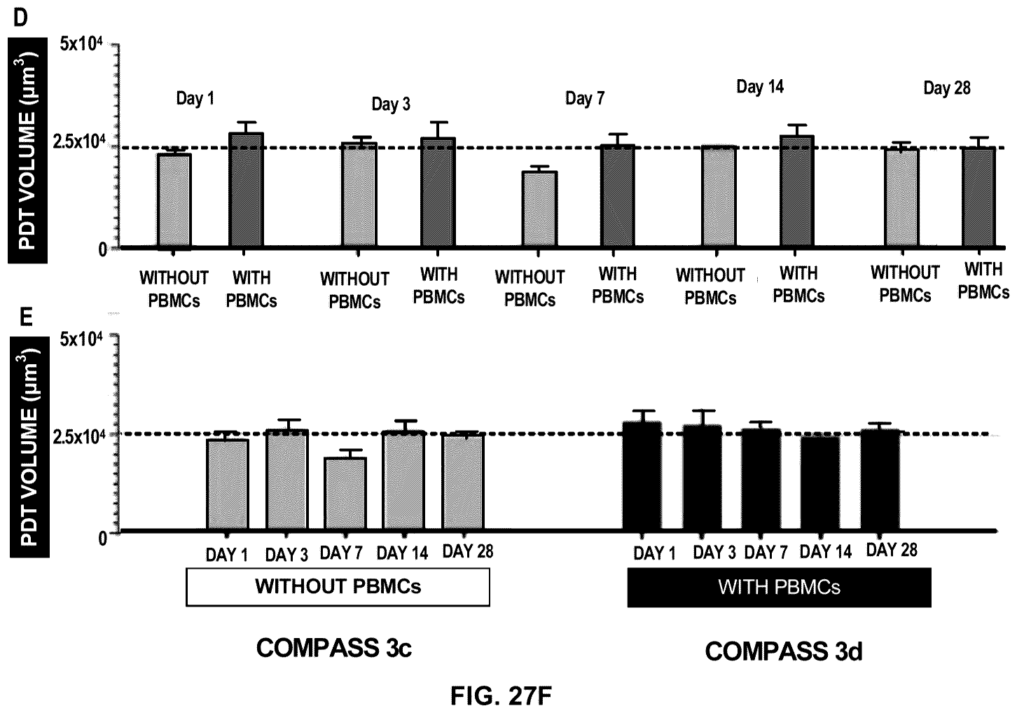

In some embodiments, the liquid-liquid interface is static. A “static” liquid-liquid interface means the blood substitute is at the bottom of the container but there is no perfusion flow occurring to force oxygen to all parts of the cell culture system. In some embodiments, the liquid-liquid interface is dynamic. (See FIGS. 26-27). A “dynamic” liquid-liquid interface means the blood substitute is at the bottom of the container but there is perfusion flow occurring to force oxygen to all parts of the cell culture system. In some embodiments, perfusion flow occurs using a pump. In some embodiments, a syringe pump circulates liquid which is oxygenated onto the tumoroid cultures instead of passive diffusion as seen in a static culture. In some embodiments, a syringe pump circulates liquid which is oxygenated onto the growth media which in turn touches the tumoroid cultures. In some embodiments, the non-immunocompetent COMPASS 3 platform showed slower PDT growth in the presence of oxygenation. In some embodiments, static models exhibit faster growth rates, plateauing at Day 7. In some embodiments, static models exhibit faster growth rates, plateauing at Day 5-10. In some embodiments, dynamic models showcase slower growth across time (such as Days 5-10) (***, p<0.001). In some embodiments, HUAS on both immuno-& non-immunocompetent COMPASS 3 showed similar growth profile for static, and non-immunocompetent models. In some embodiments, PDTs showed faster growth (than non-immunocompetent models), plateauing at Day 7 (*, p<0.042). In some embodiments, PDTs shoed growth faster (non-immunocompetent models), plateauing at Day 5-10 (*, p<0.042). In some embodiments, a static, immunocompetent PDT showed slower growth (than non-immunocompetent models), stabilizing beyond Day 7 (***, p<0.001). In some embodiments, there is an unremarkable (little) difference between growth profiles for both arms of dynamic group (non-immunocompetent models and immunocompetent models).

Broader Perspective on the Complexity of the TME Interplay: Tumor hypoxia is associated with the promotion of tumor growth and the inhibition of immune response. While the dynamic models (non-immunocompetent models and immunocompetent models) presented no differences in growth characteristics (in comparison to the static models), such variations may be associated with the aspects of TME perfusion and/or other confounding variables.

In some embodiments, COMPASS 2/3 is a reliable and versatile TME modeling platform. In some embodiments, COMPASS 2/3 shows baseline Human PDT stable growth-void of fluctuations. TME flexibility allows for a different type of and/or experiments, especially in the field of immuno-oncology. For more detail see Example 6.

In some embodiments, soft tissue sarcoma PDTs in a scaffold-based platform (COMPASS 3) were built in presence and absence of immune components, as well as the presence (dynamic) and absence (static) of oxygenation. It was observed that the oxygenation status impacts the effect of HSP90i on soft tissue sarcoma (FIG. 9-11). These studies allow the evaluation of pH-modulated and hypoxia-based therapeutics-done in combinations with small molecules, biologics and established immunotherapies (e.g., anti-PD1 or anti-CTLA4), in order to investigate the role of oxygen on these therapies. For more detail see Example 6.

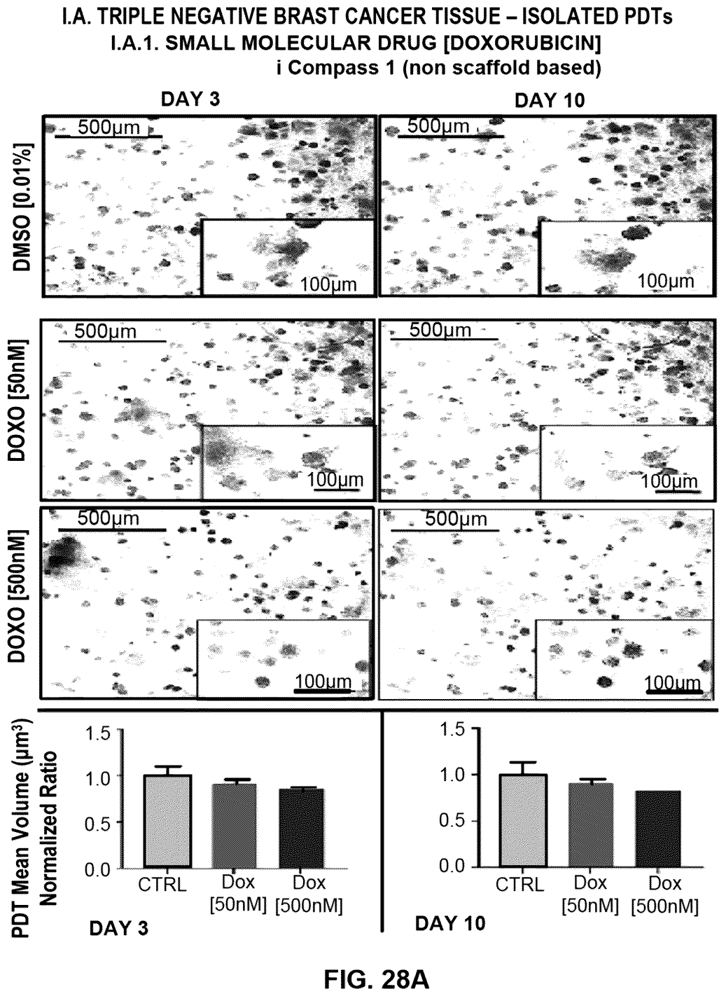

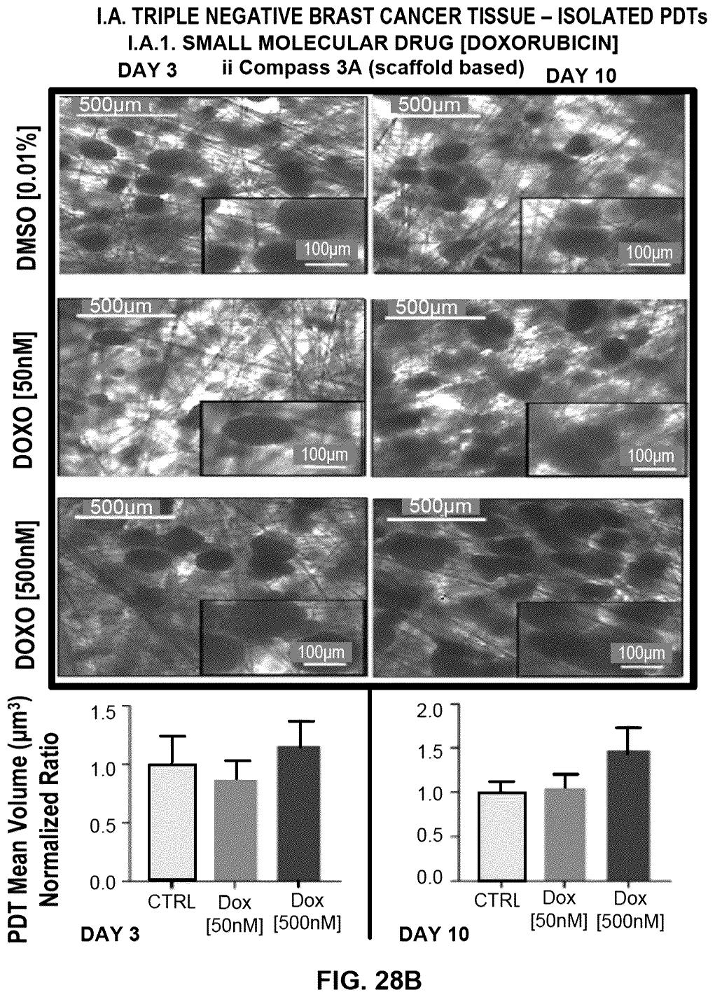

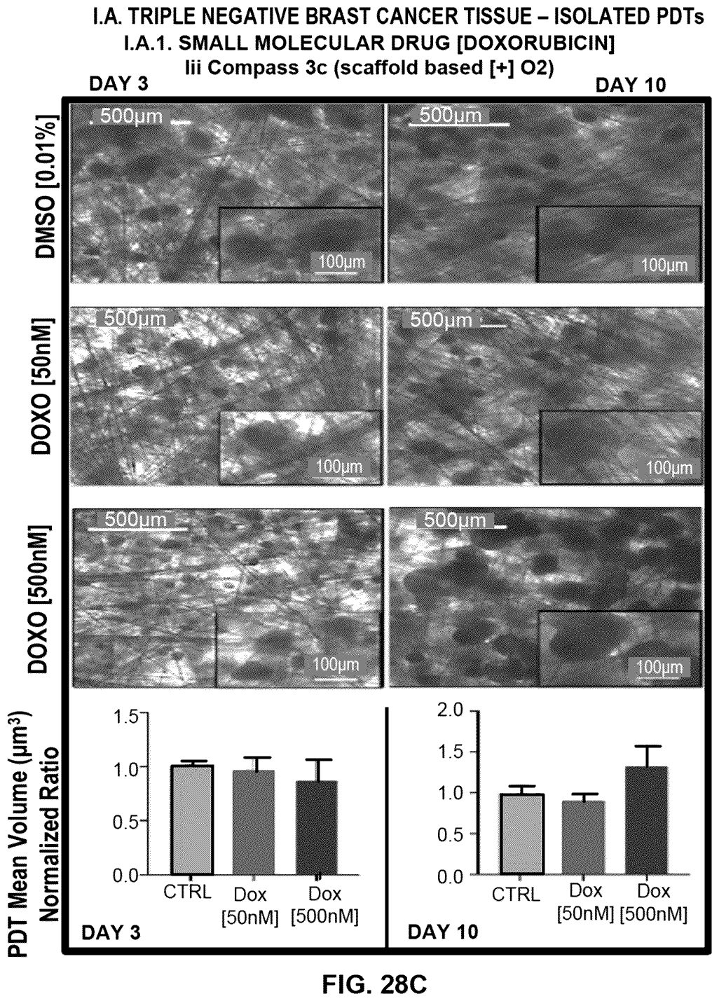

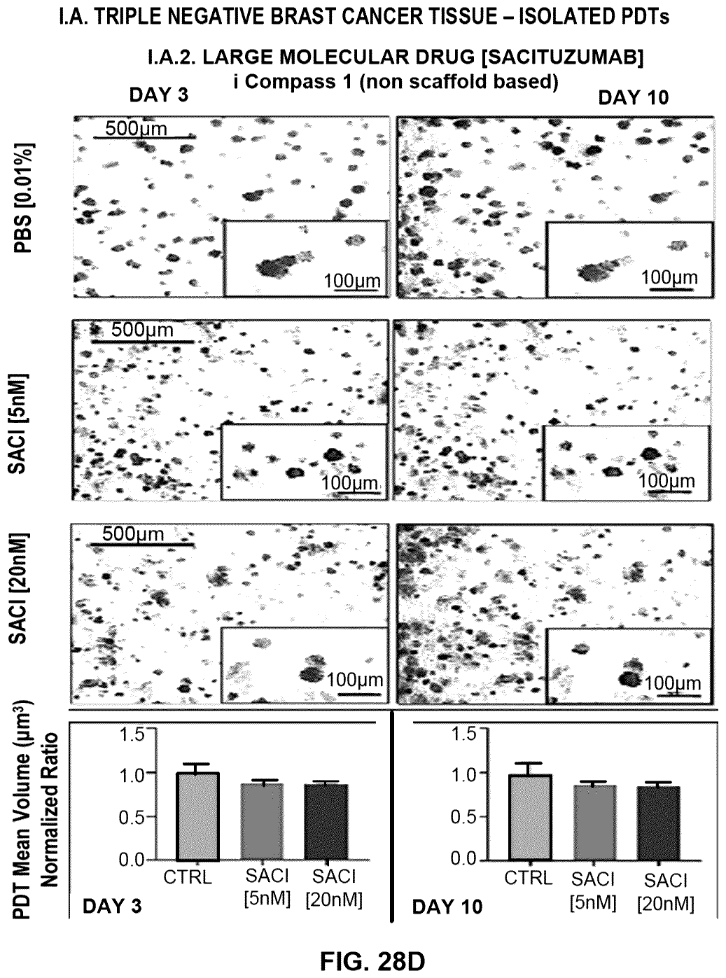

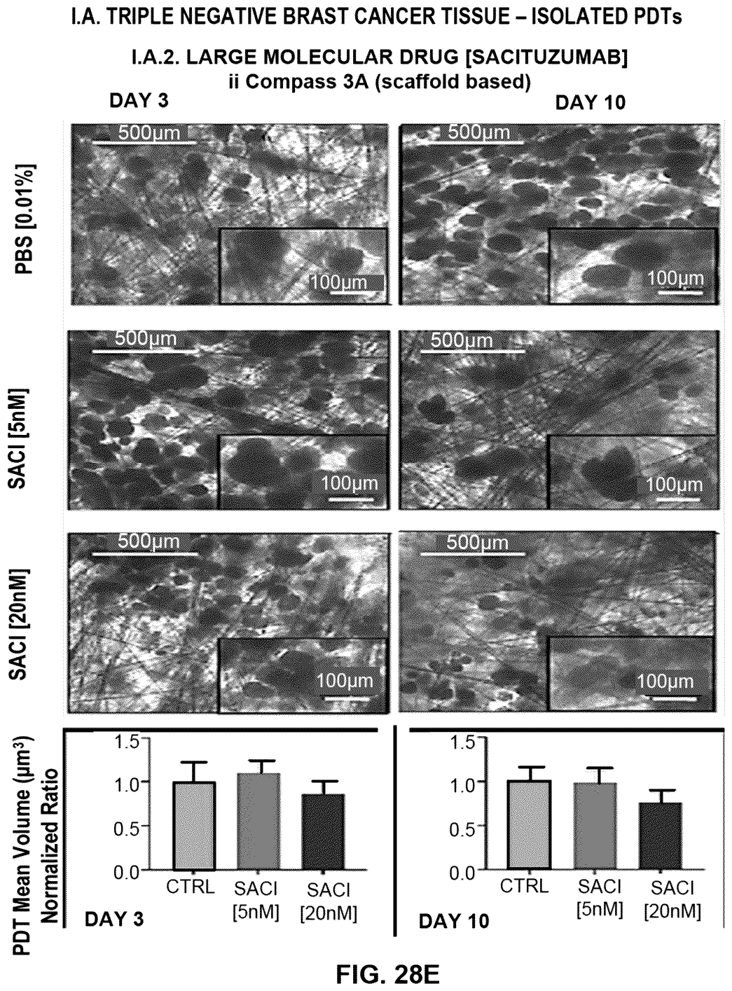

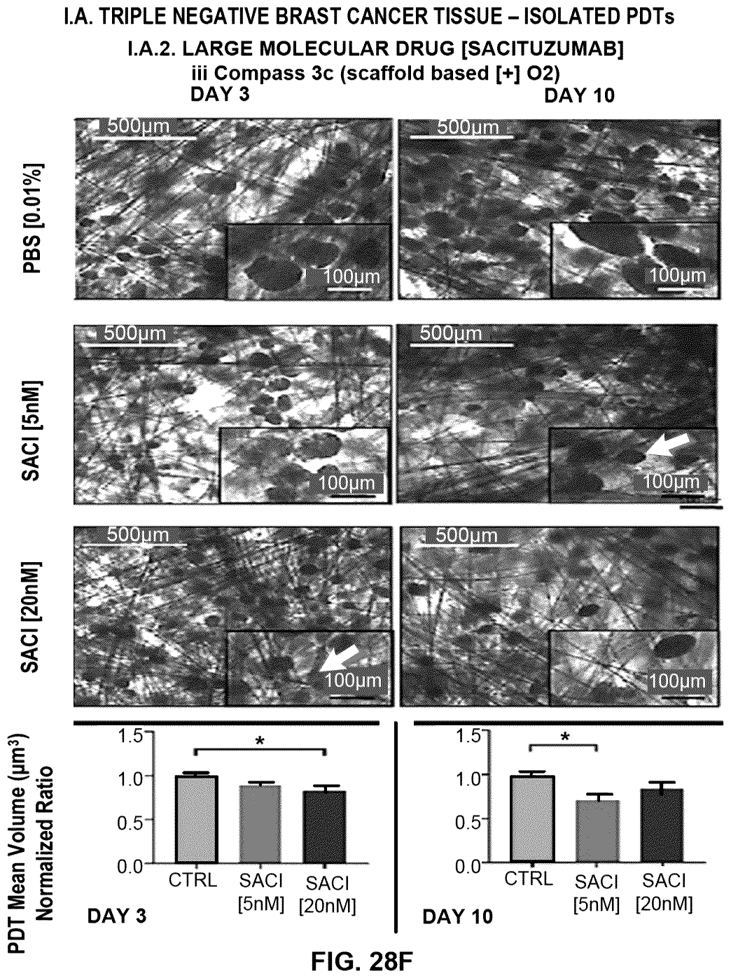

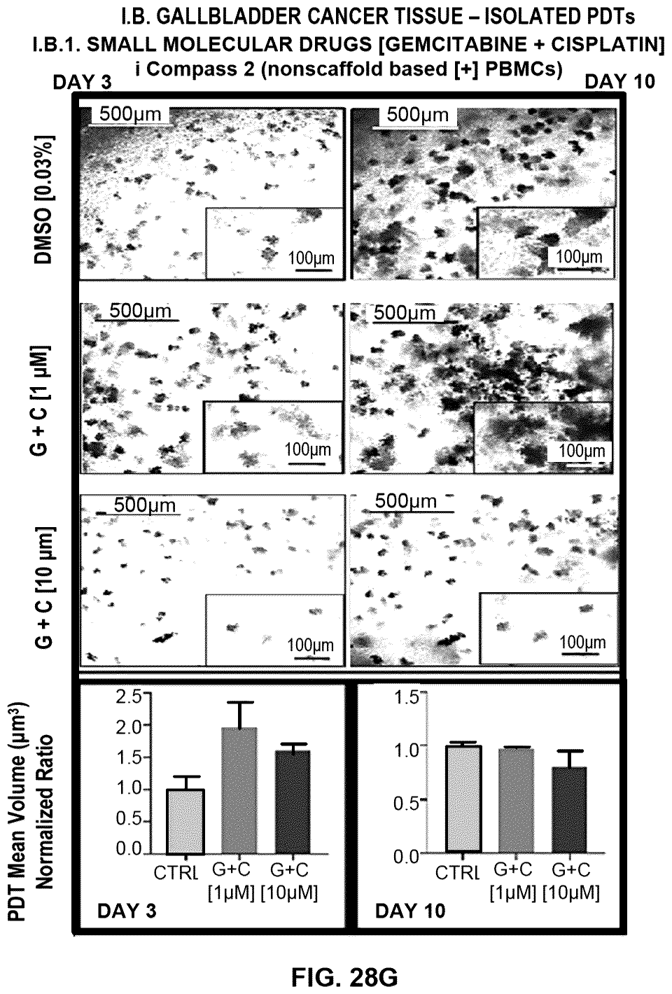

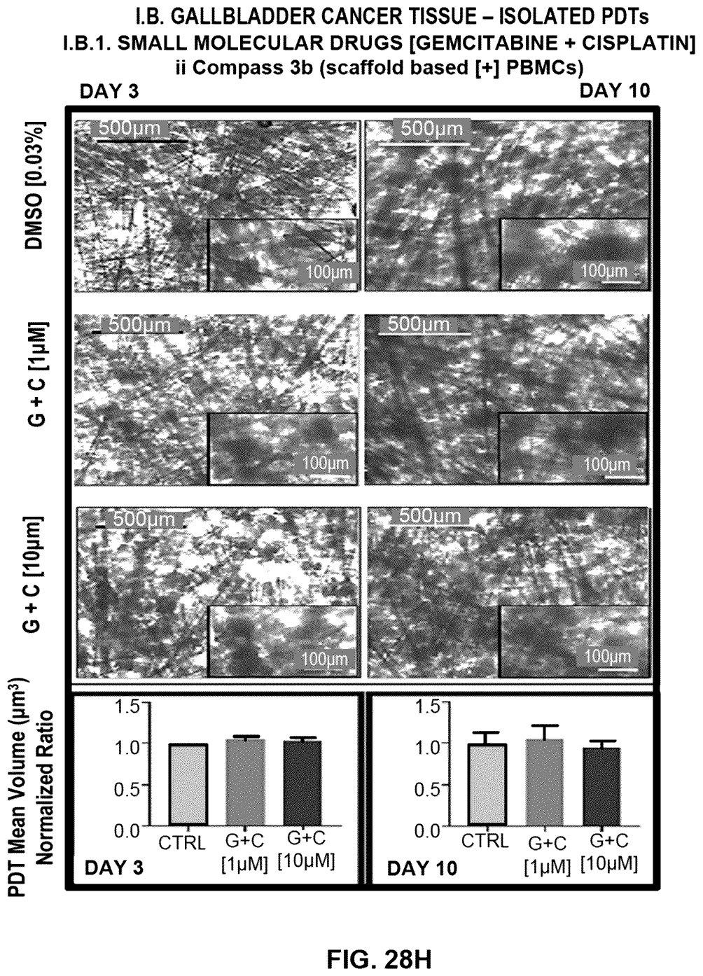

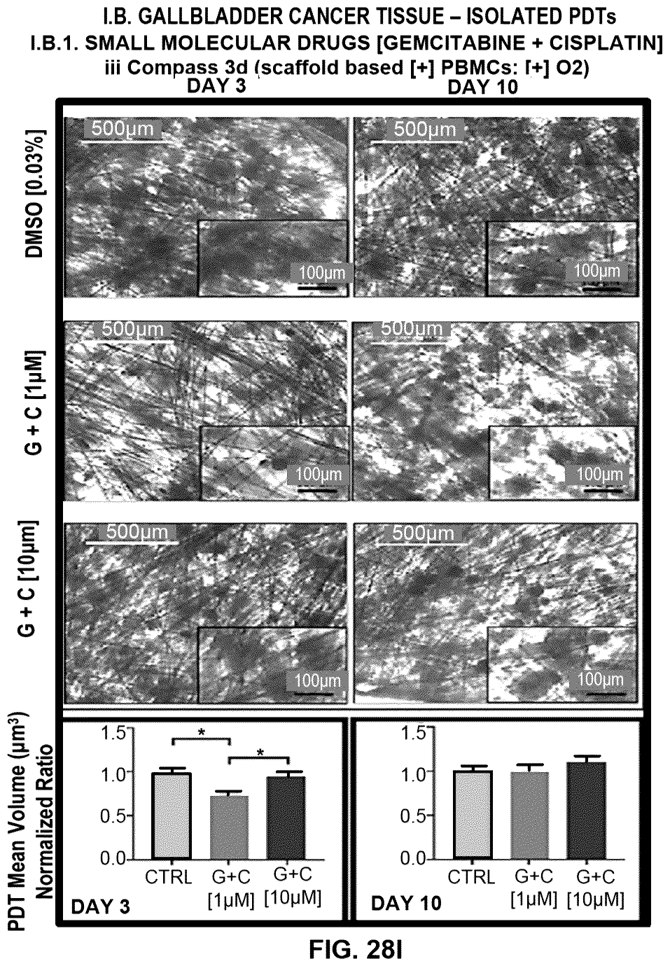

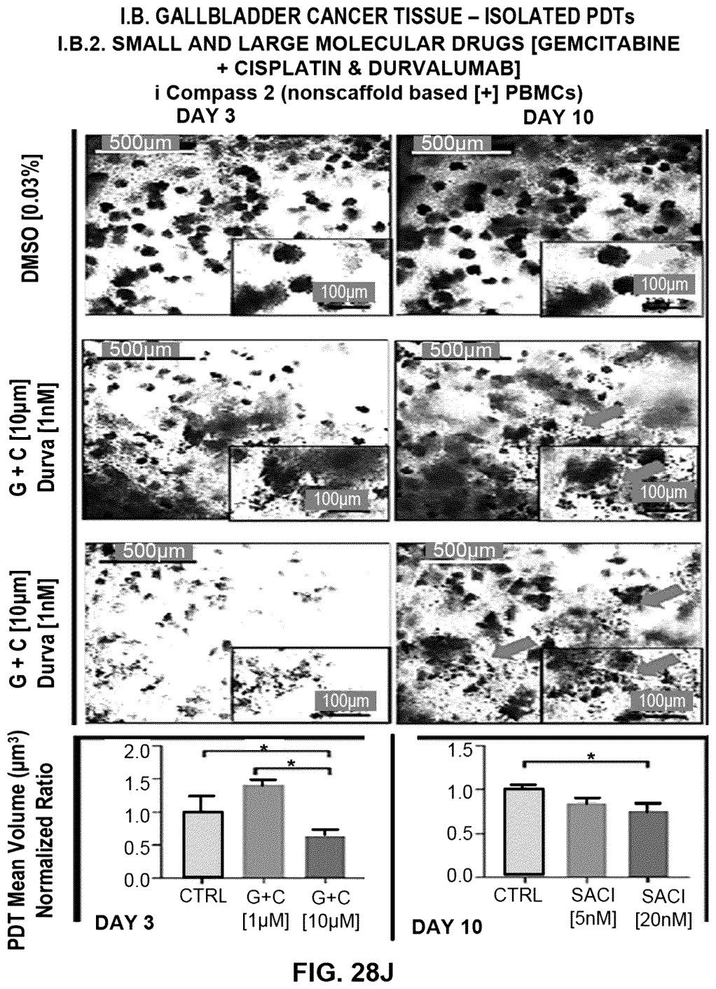

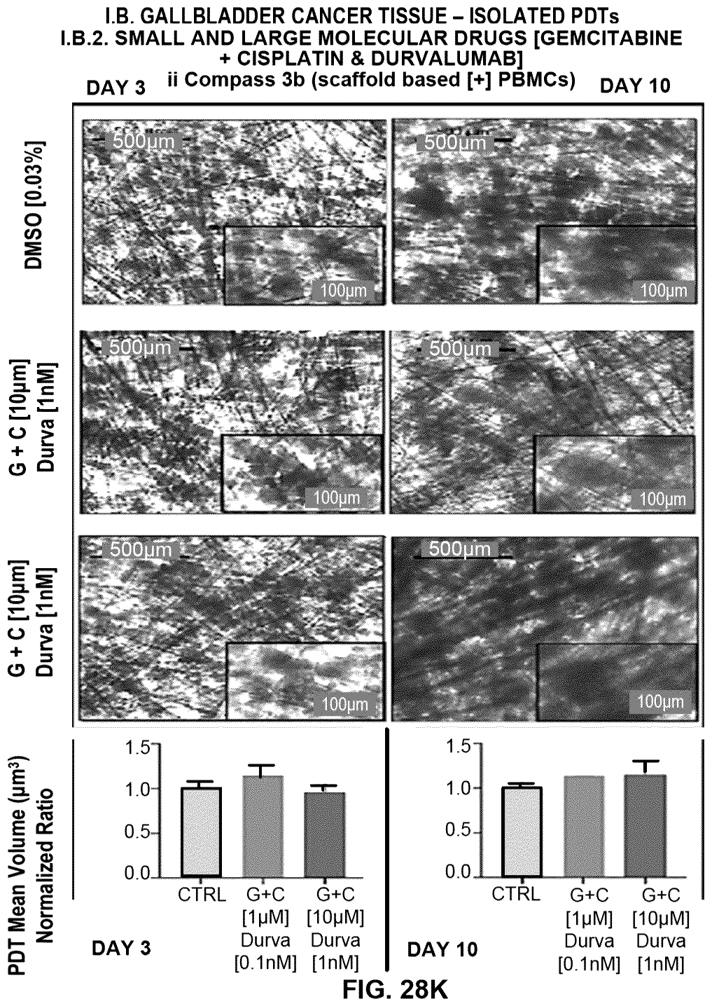

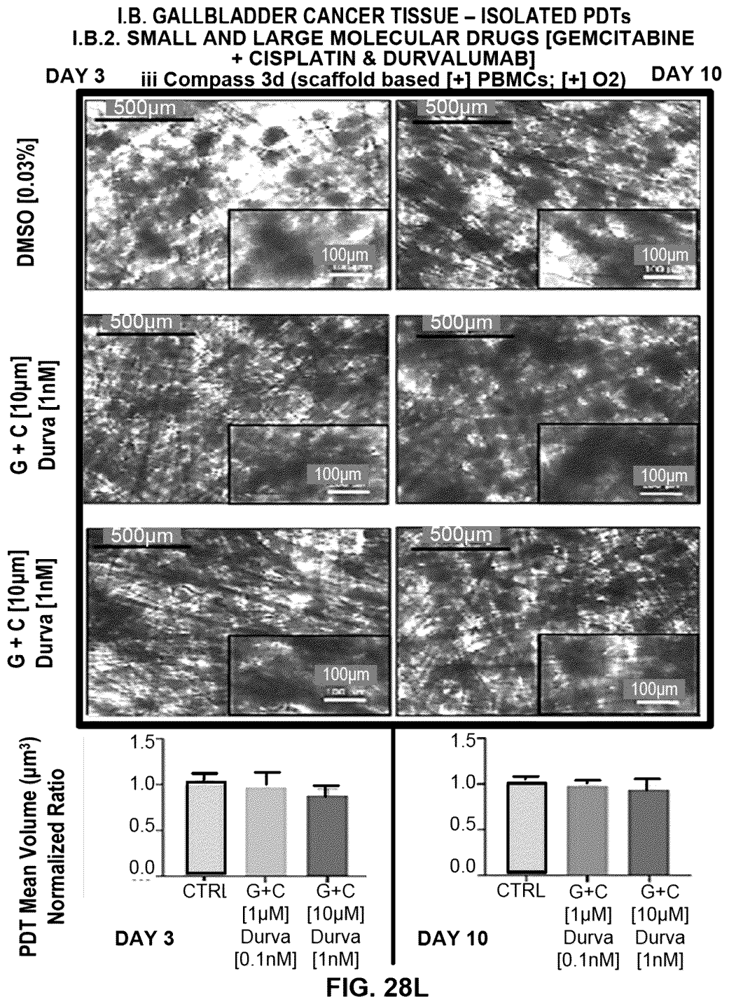

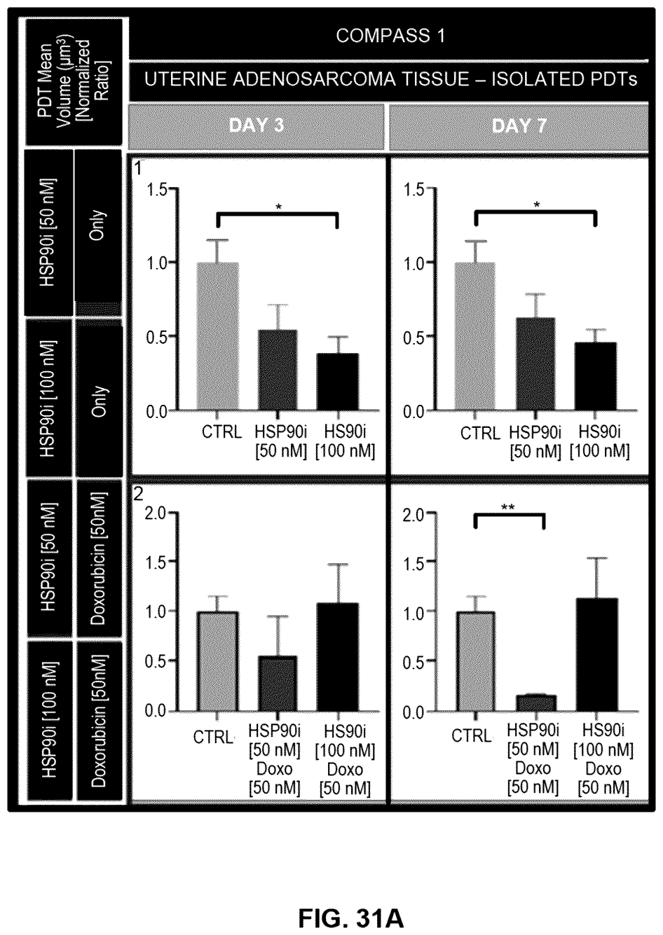

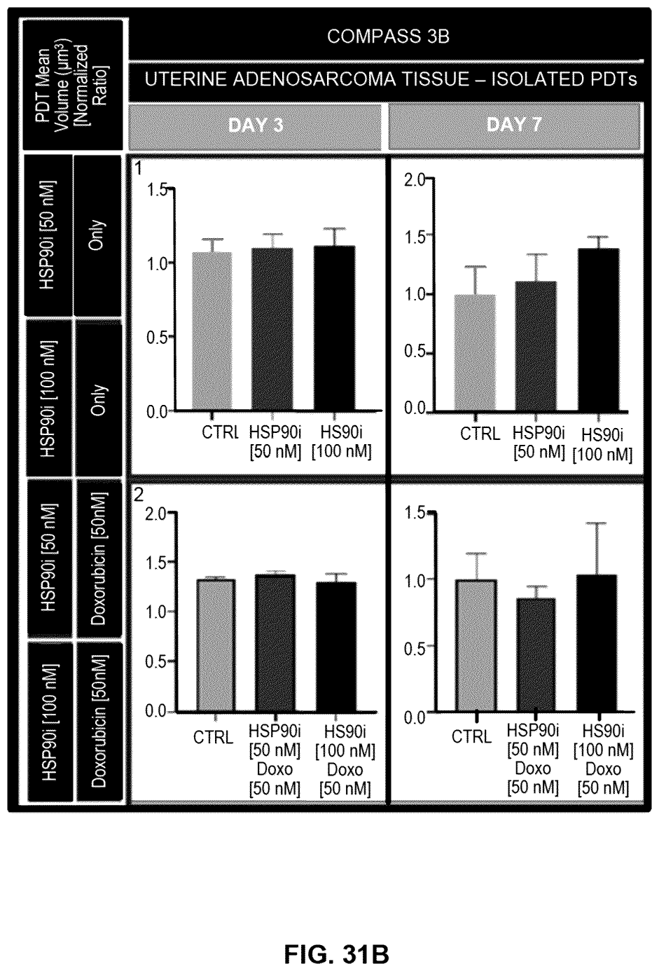

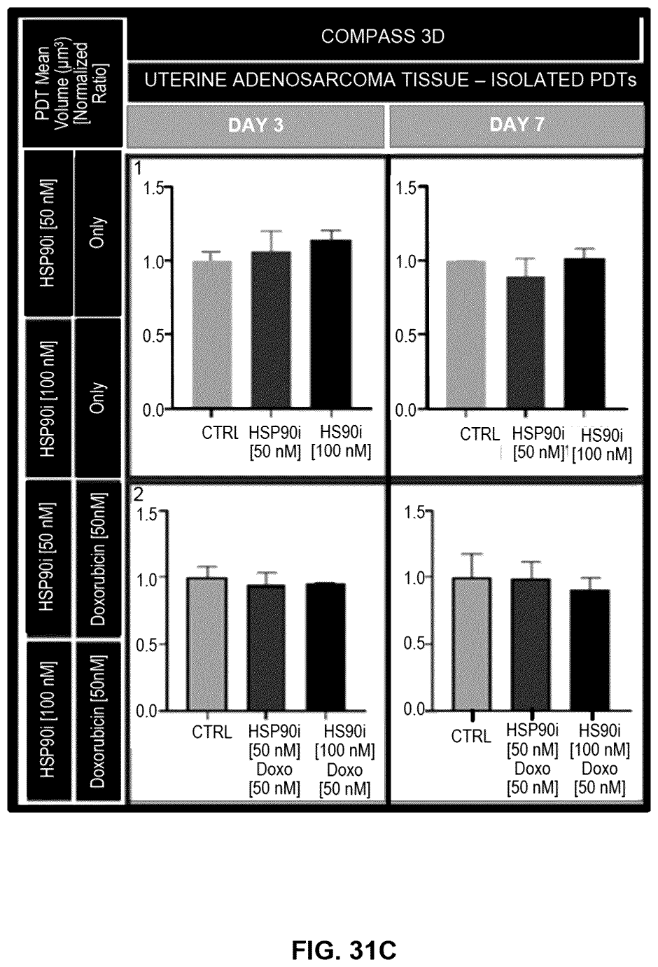

In some embodiments, the PDT generates specific tumor-immune-microenvironment models-designed appropriately for intended drug mechanism testing of already clinically-approved treatment regimens. (See FIGS. 28A-L). In some embodiments, the PDT demonstrates tumor volume reduction after treatment with a candidate agent. (See FIGS. 31A-C).

TiME-based COMPASS Model Oncotherapeutic Testing: In some embodiments, ex vivo human tumor-immune-microenvironment (TIME)-like models are used for more accurate drug testing and efficient drug discovery. In some embodiments, a three-dimensional (3D) patient-derived tumor (PDT)-based model is used for drug testing and efficient drug discovery. In some embodiments, the PDT is supplemented with variable TiME features such as structural-biochemical matrices, immunity (allogenic peripheral blood mononuclear cells (PBMCs)), and/or environmental metabolic conditions (oxygen sufficiency & insufficiency). In some embodiments, TiME-based feature diversity and COMPASS model platform versatility are used for drug testing and efficient drug discovery. In some embodiments, the Compass platform is customizable for tumor characteristic-drug action suitability.

In some embodiments, the PDT structure-aids aggressive growth and variable oxygenation conditions compared to standard or reference modalities. In some embodiments, the PDT and Compass platforms are used to study cancers with aggressive growth. Cancers for their aggressive growth include, but are not limited to, TNBC, GBC & HUAS and hypoxic microenvironmental conditions include, but are not limited to, TNBC and GBC. The system is useful for small and large molecular drugs (especially antibody-associated drugs) known for their microenvironmental condition sensitivity.

The PDTs disclosed herein showed varied PDT growth patterns and structures across tumor types. In some embodiments, more non-immunocompetent TNBC were formed than immunocompetent GBC. In some embodiments, there is a potential for small & large molecular drug penetration across model matrices.

In some embodiments, the platform and PDTs of the disclosure are used for guidance in drug design and treatment regimen optimization. In some embodiments, drug design and optimization is achieved by TiME-Drug matched optimal conditions with physiologically relevant dosing—as permitted by COMPASS' capacity to recapitulate a spectrum of necessary microenvironmental conditions.

In some embodiments, the platform and PDTs of the disclosure provide guidance towards drug potentials by re-assessing the potential of possible clinical failure of previously successful pre-clinical drugs, and/or by re-assessing the potential of possible clinical success of previously failed pre-clinical drugs.

In some embodiments, the platform and PDTs of the disclosure provide guidance to clinical success by providing drug reaction insights possibly allowing for critical patient stratification. For more detail see Example 7.

In some embodiments, the PDT-immune cell construct is dissociated and immunophenotyped by FACS.

In some embodiments, PDT-immune cell construct subjected to the COMPASS assay (Compass 1, 2, or 3) respond to the candidate agent, denoting the candidate agent is a functional treatment for a disease. The manner in which the PDT-immune cell construct responds to an agent, particularly a pharmacologic agent, including the timing of responses, is an important reflection of the candidate agent's functional treatment for a disease. In some embodiments, the candidate agent causes an increase in TIL in the PDT-immune cell construct. In some embodiments, the candidate agent cause recapitulation of checkpoint inhibition in the PDT-immune cell construct. In some embodiments, the candidate agent causes increased gene expression in the PDT-immune cell construct. In some embodiments, the candidate agent causes decreased gene expression in the PDT. In some embodiments, the candidate agent causes increased infiltrating T-cells in the PDT-immune cell construct.

Disclosed is a tumoroid methodology facilitating in vitro study of the TME through the holistic co-culture of primary tumor cells with immune cells in a media comprising, consisting of, or consisting essentially of oxygen. Disclosed is a tumoroid methodology facilitating in vitro study of the TME through the holistic co-culture of primary tumor cells with immune cells in a media comprising, consisting of, or consisting essentially of oxygen and PBMC. Such PDT-immune cell constructs provide a substantial opportunity for human in vitro drug therapy and immunotherapy modeling.

The PDT-immune cell construct system is extended to immune strategies targeting B cells, NK cells and macrophages, and/or to parallel immunotherapy concepts such as CAR T cells. The PDTs disclosed herein facilitate basic studies into the mechanisms of cancer treatments, test novel therapeutic agents and combinations, and predictively assess individualized patient responses to clinically approved immune therapies or combinations thereof.

### Identification of Agents Suitable for Treatment

Disclosed is a method for identifying an agent suitable for treating a cancer, wherein the method comprises: contacting a tumoroid-immune cell co-culture with one or more candidate agent(s), wherein the tumoroid-immune cell co-culture comprises at least one immune cell and at least one cancer/tumor cell; detecting the presence or absence of one or more change in the tumoroid-immune cell co-culture that is indicative of candidate agent suitability for treating the cancer; and identifying a candidate agent as suitable for treating the cancer if the presence or absence of one or more of said changes in the tumoroid-immune cell co-culture is detected. In some embodiments, the tumoroid-immune cell co-culture is on a matrix, supplied with oxygen from a media including oxygen, PBMC and combinations thereof. In some embodiments, the tumoroid-immune cell co-culture is without a matrix and/or without PBMC.

In some embodiments, the above method further comprises comparing the presence or absence of the one or more change of the tumoroid-immune cell co-culture with a reference organoid or reference tumoroid, and wherein the method further comprises: contacting a reference organoid/tumoroid or reference tumoroid-immune cell co-culture with the one or more candidate agents, wherein the reference organoid/tumoroid or reference tumoroid-immune cell co-culture comprises at least one immune cell and at least one cancer/tumor cell, and detecting the presence or absence of the one or more change in the reference organoid/tumoroid or reference tumoroid-immune cell co-culture that is indicative of candidate agent suitability for treating the cancer. In some embodiments, the reference organoid or reference tumoroid is on a matrix, supplied with oxygen from a media including oxygen, PBMC and combinations thereof. In some embodiments, the reference organoid or reference tumoroid is without a matrix and/or without PBMC.

The disclosure further provides a method for identifying an agent suitable for treating an immune disease, wherein the method comprises: contacting an organoid/tumoroid-immune cell co-culture with one or more candidate agents, wherein the organoid/tumoroid-immune cell co-culture comprises diseased immune cells and at least one cancer/tumor cell, detecting the presence or absence of one or more change in the organoid/tumoroid-immune cell co-culture that is indicative of candidate agent suitability for treating the immune disease, and identifying a candidate agent as suitable for treating the immune disease if the presence or absence of one or more of said changes in the organoid/tumoroid-immune cell co-culture is detected. In some embodiments, the tumoroid-immune cell co-culture is on a matrix, supplied with oxygen from a media including oxygen, PBMC and combinations thereof. In some embodiments, the tumoroid-immune cell co-culture is without a matrix and/or without PBMC.

In some embodiments, the above method further comprises comparing the presence or absence of the one or more change of the organoid/tumoroid-immune cell co-culture with a reference immune cell (e.g. from a control patient lacking the immune disease), and wherein the method further comprises: contacting a reference organoid/tumoroid-immune cell co-culture with the one or more candidate agents, wherein the reference organoid/tumoroid-immune cell co-culture comprises at least one immune cell and at least one organoid, and detecting the presence or absence of the one or more change in the reference organoid/tumoroid-immune cell co-culture that is indicative of candidate agent suitability for treating the immune disease. In some embodiments, the reference organoid or reference tumoroid is on a matrix, supplied with oxygen from a media including oxygen, PBMC and combinations thereof. In some embodiments, the reference organoid or reference tumoroid is without a matrix and/or without PBMC.

Accordingly, disclosed is a method of testing a candidate compound for efficacy and/or safety, wherein the method comprises: contacting a tumoroid-immune cell co-culture with one or more candidate agent(s), wherein the tumoroid-immune cell co-culture comprises at least one immune cell and at least one cancer/tumor cell; detecting the presence or absence of one or more change in the tumoroid-immune cell co-culture, wherein the presence or absence of one or more change is indicative of efficacy and/or safety of the candidate compound. In some embodiments, the tumoroid-immune cell co-culture is on a matrix, supplied with oxygen from a media including oxygen, PBMC and combinations thereof. In some embodiments, the tumoroid-immune cell co-culture is without a matrix and/or without PBMC.

Also provided is a method for testing a therapeutic agent, wherein the method comprises:
contacting an organoid/tumoroid-immune cell co-culture with one or more candidate agents, wherein the organoid/tumoroid-immune cell co-culture comprises at least one immune cell and at least one cancer/tumor cell, detecting the presence or absence of one or more change in the organoid/tumoroid-immune cell co-culture that is indicative of therapeutic efficacy, and identifying a candidate agent as a therapeutic agent if the presence or absence of one or more of said changes in the organoid/tumoroid-immune cell co-culture is detected.

### Screening Methods for Agents Suitable for Treatment

Disclosed are methods and culture systems for screening candidate agents for an activity of interest. In these methods, candidate agents are screened for their effect on cells in the PDT-immune cell construct described herein, including without limitation the cancer/tumor cells and/or immune cells associated with the tumor.

The effect of a candidate agents, e.g., a drug therapeutic, an immunotherapeutic agent, nucleic acid, polypeptide, small molecule, large molecule, and/or virus, is determined by adding the candidate agents to the cells of the cultured explants (PDT-immune cell construct) as described herein, usually in conjunction with a control culture of cells lacking the candidate agents. The effect of the candidate agents is then assessed by monitoring one or more output parameters/readout. Output parameters are quantifiable components of the PDT-immune cell construct, particularly components that are accurately measured, in some instances in a high throughput system. For example, an output parameter is the growth, volume, differentiation, survival, gene expression, proteome, phenotype with respect to markers etc. of the PDT-immune cell construct, e.g. any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. Whilst most parameters will provide a quantitative output parameter, in some instances a semi-quantitative or qualitative output parameter will be acceptable. Output parameters may further include a single determined value, or may include mean, median value, or the variance, etc. Characteristically a range of output parameter values will be obtained for each parameter from a multiplicity of the same assays. Variability is expected and a range of values for each of the set of output parameters will be obtained using standard statistical methods with a common statistical method used to provide single values.

In some embodiments, candidate cells and/or candidate agents are added to the cells within the intact organoid. In other embodiments, the PDT-immune cell constructs are dissociated, and candidate agents are added to the dissociated cells. In some embodiments, the PDT-immune cell constructs are freshly isolated, cultured, genetically altered, etc. In some embodiments, the PDT-immune cell constructs are environmentally induced variants of clonal cultures: e.g., split into independent cultures and grown into organoids/tumoroids under distinct conditions, for example with or without pathogen; in the presence or absence of other cytokines; in the presence or absence of other candidate agents; or combinations thereof.

The disclosure concerns co-cultures of organoids/tumoroids and immune cells (‘organoid/tumoroid-immune cell co-cultures’) and/or co-cultures of disease organoids (such as tumoroids) and immune cells (‘tumoroid-immune cell co-cultures’), and their use for investigating the physiology of diseases and/or the suitability of candidate agents for treating diseases. Suitability for treating a disease may comprise efficacy for treating the disease and/or safety for treating the disease. Diseases of particular interest include cancer and immune diseases.

The methods of the disclosure have high-throughput (HTP) capacity. In some embodiments, the method of the disclosure is performed on 96-well plates and/or on 384-well plates.

In some embodiments, the candidate agent to be screened has an unknown suitability for treatment. In some embodiments, the candidate agent to be screened is known to be a suitable agent for treatment.

In some embodiments, the one or more candidate agents are of known suitability for treating a first cancer and unknown suitability for treating a second cancer (wherein the first and second cancer are different), with screening comprising, consisting of, or consisting essentially of identifying a subset of the one or more candidate agents as suitable agents for treating the second cancer.

In some embodiments, the screening approach identifies agents suitable for treating cancer at the population level, rather than at the level of sub-populations. In other embodiments, the screening approach identifies agents suitable for treating cancer at the level of sub-populations. In some embodiments, the screening approach is used to identify agents suitable for treating cancer at the level of individual patients.

### Contacting Step

The contacting step may involve exposing the organoid/tumoroid-immune cell co-culture to therapeutic levels of a known or unknown therapeutic. Typically, one or more agent(s) will be dissolved in solution to a (predicted) therapeutically effective concentration and administered to the tumoroid-immune cell co-culture by injection (or other appropriate administration) into a vessel in which the tumoroid-immune cell co-culture is maintained. In some embodiments, the one or more candidate agent(s) is added to the media of the tumoroid-immune cell co-culture. In some embodiments, the one or more candidate agent(s) is added directly to the cells in the coculture itself. In some embodiments, the one or more candidate agent(s) is added directly to a portion of the cells in the coculture itself.

### Detecting Step

The detecting step may involve a step of detecting the presence or absence of one or more changes in the organoid/tumoroid-immune cell co-culture that are indicative of candidate agent suitability for treatment.

Any biochemical, genetic, phenotypic, or phenomenological change in the tumoroid-immune cell co-culture can be detected. In some embodiments, the one or more changes are in one or more disease biomarkers, such as cancer biomarkers or immune biomarkers. In some embodiments, the one or more change may include a reduction in cell viability, a reduction in cell proliferation, an increase in cell death, a change in cell or PDT-immune cell construct size (bigger or smaller), a change in cell or PDT-immune cell construct volume (bigger or smaller), a change in cell motility, dissociation or disruption of the intact/compact epithelial cell layer (i.e. cells dissociate from the compact epithelial cell layer), change in production of cytokines and cytotoxic molecules by co-cultured immune cells, a change in the expression of one or more genes, a change in expression of a protein and combinations thereof.

Detection is performed using any suitable laboratory method known to the skilled person. In some embodiments, detecting one or more changes comprises a cellular proliferation assay, a viability assay, flow cytometric analysis, ELISA, analysis of gene expression and/or cellular imaging, analysis of protein expression and combinations thereof.

A reduction in cell viability can be detected by CellTiter Glo Luminescent Cell Viability Assay kit (Promega), intracellular flow cytometric staining for active Caspase 3 (BD), or positive stain for death cells. Positive strain for death cells includes non-cell membrane permeable DNA stains such as NucRed Dead 647 ReadyProb.

An increase in cell death can be detected by brightfield imaging.

The detection step may comprise identifying a change of a particular magnitude, of change. The detection step comprises different types of readouts. Types of readouts include growth analysis, functional assays, proteomic characterization, cellular level analysis, tissue level analysis, immunological imaging, immunological assays, and combinations thereof.

Growth Analysis can include tumor size reduction at endpoint; Interim readouts for growth usually Day 0, Day 3, Day 7, Day 10, and Day 14.

Functional Assays can include apoptosis, proliferation, migration, and/or real time PCR.

Genomic Characterization can include biomarkers, bulk & single cell RNA seq, pre-/post-RNA seq of PBMCs/tumoroids, gene expression for longitudinal studies, orthogonal readouts at different time points.

Proteomic Characterization can include IFC analysis, IHC analysis, live cell IFC for cell surface markers, fixed cell imaging for non-cell surface markers.

Cellular Level Analysis can include cell migration followed and quantified using phase contrast microscopy over time; high content screening for tumor changes over treatment/time.

Tissue Level Analysis can include serial sectioning, H&E staining, embedding, sectioning and downstream processing via IHC and IF.

Immunological Imaging can include TIL infiltration quantified. Followed by identification of cell surface markers using a fluorescent label, three color staining and used immunofluorescence which could be quantified, FACS.

Immunological Assays can include cytokine arrays, multiplex IFC effect on TILs, labeling, separation, and IFC of TILs, CD4 and CD28, immunotyping over time, target engagement and changes in immune ME, assays for cytokines with longitudinal studies.

### Identifying Step

The identifying step comprises identifying a candidate agent as suitable for treating cancer. The identifying step comprises identifying a candidate agent as suitable for treating cancer based on a readout or change as described above.

### Comparing Step

The comparing step can comprise comparing the organoid/tumoroid or tumoroid-immune cell co-culture with a control, which may or may not be associated with the identifying step. In some embodiments, this involves comparing the presence or absence or magnitude of one or more changes of the organoid/tumoroid-immune cell co-culture with a reference organoid or reference tumoroid. In some embodiments comparing the presence or absence or magnitude of one or more changes of the organoid/tumoroid-immune cell co-culture with a reference organoid or reference tumoroid may further comprise: contacting a reference organoid or reference tumoroid-immune cell co-culture with the one or more candidate agents, wherein the reference organoid or reference tumoroid-immune cell co-culture comprises at least one immune cell and at least one tumor cells, and detecting the presence or absence of the one or more change in the reference organoid or reference tumoroid-immune cell co-culture that is indicative of candidate agent suitability for treating the cancer.

In some embodiments, a candidate agent is identified as a suitable agent if the presence or absence of a biochemical, genetic, phenotypic, or phenomenological change in the tumoroid-immune cell co-culture is detected but not in the reference co-culture.

In some embodiments, the reference organoid/tumoroid or reference tumoroid-immune cell co-culture is used as a control, such as a negative control or a positive control.

### Selecting Step

The selecting step comprises selecting a candidate agent as suitable for treating cancer. Selecting is distinct from identifying, as selecting may involve considerations such as the presence or absence or magnitude of the one or more biochemical, genetic, phenotypic, or phenomenological change of the provided method. For example, selecting may comprise additional considerations such as agent bioavailability, applicability to a patient sub-population, or agent delivery mechanisms.

In some embodiments, this step is the final step of the method of the disclosure. In other embodiments, further steps are envisaged. For example, methods of the disclosure may further comprise the step of using the selected candidate agent in treatment.

### Candidate Agents

Any candidate agent can be tested according to the method of the disclosure. This includes any biological, chemical, physical, or other agent, or multiple agents administered simultaneously or in sequence.

In some embodiments, the candidate agent is a nucleic acid, polypeptide, small molecule, large molecule, viruses, and/or combinations thereof.

Candidate agents of interest for screening include known and unknown compounds that encompass numerous chemical classes, primarily organic molecules, for example antibodies, cytokines, genetic sequences, etc. Disclosed herein is the evaluation of candidate agents to predict patient responsiveness to candidate agents.

In some embodiments, the candidate agents are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g., human, rodent, rabbit, cow, sheep, pig, dog, other mammal, chicken, other avians, etc., are considered to be “antibodies.” Antibodies utilized in the present invention are either polyclonal antibodies or monoclonal antibodies. Antibodies are typically provided in the media in which the cells are cultured.

In some embodiments, candidate agents are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural, or synthetically produced libraries and compounds are readily modified through conventional chemical, physical and biochemical means, and are used to produce combinatorial libraries. Known pharmacological agents are subjected to directed or random chemical modifications, such as acylation, alkylation, esterification, amidification, etc. to produce structural analogs.

In some embodiments, candidate agents are screened for biological activity by adding the agent to at least one and usually a plurality of PDT-immune cell constructs, usually in conjunction with PDT-immune cell constructs not contacted with the candidate agent. The change in parameters in response to the candidate agent is measured, and the result/readout evaluated by comparison to reference cultures, e.g., in the presence and absence of the agent, obtained with other agents, etc.

In some embodiments, the candidate agents are added in solution, or readily soluble form, to the medium of the PDT-immune cell construct in culture. In some embodiments, the candidate agents are added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the candidate agent added. In some embodiments, the first fluid is passed over the PDT-immune cell construct, followed by the second. In some embodiments, the first fluid is passed over the PDT-immune cell construct simultaneously with the second. In some embodiments, a bolus of the candidate agent(s) is added to the volume of medium surrounding the PDT-immune cell construct. In some embodiments, a bolus of the candidate agent(s) is added directly to the PDT-immune cell construct. In some embodiments, the candidate agent(s) is injected into the PDT-immune cell constructs, e.g., into the lumen of the PDT-immune cell constructs. In some embodiments, the overall concentrations of the components of the culture medium should not change significantly with the addition of the bolus, or between the two solutions in a flow-through method.

In some embodiments, the candidate agent includes additional components, such as preservatives. In some embodiments, the candidate agent includes a physiologically acceptable carrier, e.g., water, ethanol, DMSO, etc. In some embodiments, the candidate agent consists, or consists essentially of the candidate agent itself.

In some embodiments, a plurality of assays is run in parallel with different candidate agent concentrations to obtain a differential response to the various concentrations. As known in the art, determining the effective concentration of a candidate agent typically uses a range of concentrations resulting from 1:1, 1:5, 1:10, 1:20, 1:30, 1:40, 1:50, 1:60, 1:70, 1:80, 1:90, 1:100, 1:500, 1:1000, 1:10,000 or other log scale, dilutions. In some embodiments, the concentrations are further refined with a second series of dilutions, if necessary. In some embodiments, one of these concentrations serves as a negative control, i.e., at zero concentration or below the level of detection of the agent or at or below the concentration of candidate agent that does not give a detectable change (biochemical, genetic, phenotypic, or phenomenological change).

In some embodiments, a candidate agent is screened for activity that is anti-tumorigenic (i.e., inhibiting cancer initiation) or anti-tumoral (i.e., inhibiting cancer progression, e.g., proliferation, invasion, metastasis). In such embodiments, the PDT-immune cell construct comprises cancer cells, including cells suspected of being cancer cells or cancer stem cells. Assessment of anti-tumor activity may include measurements of one or more parameters including PDT-immune cell construct growth, the rate or extent of cell proliferation, the rate or extent of cell death, etc. Assessment of anti-tumor activity may also include analysis of markers of immune cell activation (which include but are not limited to IFN-γ, granzyme, perforin, etc.), expansion or alteration of immune cell populations (T, B, NK, monocyte/macrophage, dendritic cells, myeloid-derived suppressor cells), tumor cell death tumor phagocytosis and the like. Immune cells could be isolated and/or analyzed by any number of means including FACS, CyTOF, MIBI, multiplexed immunohistochemistry, quantitative RT-PCR, Luminex or others.

The candidate agents undergoing testing for the suitability of treating cancer, is selected from one or more of the following therapeutic classes: immunotherapeutic, tumor-specific peptides, checkpoint inhibitors, alkylating agent, antimetabolite, metabolic agonist, metabolic antagonist, plant alkaloid, mitotic inhibitor, antitumor antibiotic, topoisomerase inhibitor, radiotherapeutics, chemotherapeutics, antibodies, photosensitizing agent, stem cell transplant, vaccine, cytotoxic agent, cytostatic agent, tyrosine kinase inhibitor, proteasome inhibitor, cytokine, interferon, interleukin, intercalating agent, targeted therapy agent, small-molecule drug, hormone, steroid, cellular therapeutic, viral vector, and nucleic acid therapeutic. In some embodiments, the candidate agents are tumor-specific peptides, checkpoint inhibitors, immune-therapeutics. In some embodiments, the candidate agents are more preferably immune-therapeutics, for example chimeric antigen receptor (CAR)-T cell therapeutics, therapeutic TCR transgenic T cells, or neoantigens. In some embodiments, the candidate agents are associated with antibody-dependent cell-mediated cytotoxicity (ADCC) or antibody-dependent cellular phagocytosis (ADCP).

In some embodiments, the candidate agents are chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1l and calicheamicin ω1l (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chlorambucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretinoic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG12 antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A: 636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc.); and mAb 806 or humanized mAb 806 (Johns et al., J. Biol. Chem. 279 (29): 30375-30384 (2004)). The anti-EGFR antibody are conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl) methoxy]phenyl]-6 [5 [[[2 methylsulfonyl)ethyl]amino]methyl]-2-furanyl]-4-quinazolinamine).

In some embodiments, the candidate agents are “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNER); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

In some embodiments, the candidate agents are dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

In some embodiments the candidate agent is an immunotherapeutic agent, including without limitation checkpoint inhibitors; agonists of immune costimulatory molecules; antibodies specific for tumor antigens, which antibodies may activate effector functions on immune cells; activators of innate immune responses; CAR-T cells; and combinations thereof.

### Personalized Medicine

In some embodiments, functional in vitro assays are provided for determining patient specific responsiveness to candidate agents within a clinically actionable time frame.

Disclosed is a method for determining responsiveness of a patient's tumor to a candidate agent, the method comprising, consisting of, or consisting essentially of: obtaining a tumor tissue sample comprising, consisting of, or consisting essentially of none immune cells (such as TILs, parenchymal, and/or stromal cells) and immune cells associated with the tumor; culturing the tumor tissue sample in gas-liquid interface of Oxygen to provide a patient specific tumoroid (PDT) with non-immune and immune cell elements; contacting the PDT with a candidate agent for a period of time sufficient to modulate a change (biochemical, genetic, phenotypic or phenomenological change); determining the effect (a magnitude of change or comparison to a reference/control tumoroid) of the candidate agent on PDT-immune cell construct activity wherein reduction in cell viability, a reduction in cell proliferation, an increase in cell death, a change in cell or organoid size, a change in cell motility, dissociation or disruption of the intact/compact epithelial cell layer (i.e. cells dissociate from the compact epithelial cell layer), change in production of cytokines and cytotoxic molecules by co-cultured immune cells, a change in the expression of one or more genes, a change in expression of a protein and combinations thereof relative to a control in the absence of the agent is indicative the patient is responsive to the candidate agent. In some embodiments, the PDT is exposed to a second media and the first and second media form a liquid-liquid interface.

Disclosed are compositions and methods for in vitro culture systems of human solid tumors as 3-dimensional patient derived tumoroids (PDT) that recapitulate the cellular architecture and ultrastructure of the tumor sample from which they were derived. In some embodiments, the PDT-immune cell construct comprises immune cells such as TILs, parenchymal and stromal elements. In some embodiments, the compositions and methods provide screening assays useful as a functional prognostic to predict a patient's response to a candidate agent, including but not limited to immunotherapies. In some embodiments, an individual determined to be responsive to a candidate agent is treated accordingly, e.g., by administering an effective dose of a candidate agent. The preclinical efficacy of the candidate agent can also be determined.

In some embodiments, screening assays are provided. In such assays, a PDT-immune cell construct culture is initiated with a solid tumor sample. In some embodiments, the patient sample comprises tumor and non-tumor cells. In some embodiments, the patient sample comprises tumor and immune cells. In some embodiments, the patient sample comprises non-immune and immune cells. In some embodiments, the patient sample comprises tumor, non-tumor, non-immune, and immune cells. In some embodiments, the patient sample comprises tumor cells, epithelial, tumor infiltrating lymphocytes, parenchymal and stromal elements, immune cells, and combinations thereof. In some embodiments, the patient sample comprises normal (e.g., non-tumor) cells.

In some embodiments, the PDT-immune cell construct culture is contacted with a candidate agent of interest for a period of time (1 day, 2 days, 3 days, 4 days, 5 days, 6 days, 7 days, 8 days, 9 days, 10 days, 14 days, 24 days, 40 days, 50 days, 60 days, 80 days, 100 days, 1-7 days, 1-14 days, 1-21 days, 1-30 days, 1-40 days, 1-50 days, 1-100 days) sufficient to allow an effect on the PDT-immune cell constructs, and the effect on the tumor and/or immune cells associated with the tumor are assessed.

The effectiveness of the agent is monitored by analysis of the immune cells present in the PDT-immune cell construct, e.g., by detecting changes in expression of markers associated with cancer or immune activation, including but not limited to IFNG, GZMB, PRF1, etc. Effectiveness of the agent are functionally measured by the response of immune cells against the PDT tumor cells. The assay is completed in a clinically actionable time frame, e.g., within about 3 days, within about 5 days, within about 7 days, within about 10 days, within about 14 days, within about 24 days, within about 30 days, within about 40 days, within about 60 days e.g., from the time that the agent is brought into contact with the PDT-immune cell construct.

In some embodiments, one or more candidate agents that are of known suitability for treatment, and/or identifying the one or more candidate agents as suitable agents in a particular patient are tested.

In some embodiments, the both the tumoroid-immune cell co-culture and a reference organoid/tumoroid or reference tumoroid-immune cell co-culture are derived from the same patient for whom the suitability of candidate agents for treating cancer is being identified.

In some embodiments, normal (e.g., non-tumor) epithelial cells and tumor epithelial cells are derived from a single tissue in a single patient, and organoid-immune cell co-cultures and tumoroid-immune cell co-cultures from these cells is obtained and the tumoroid-immune cell co-culture's response to candidate agents is assessed. In some embodiments, a patient for whom a candidate agent has been identified as being suitable for treating cancer, may subsequently be treated with the candidate agent so-identified.

In some embodiments, the methods disclosed herein include determining the ratio of expression levels of one or more cell gene signatures in a PDT-immune cell construct between gene sets to further identify a cancer patient for treatment with a candidate agent or who may have the likelihood of benefiting from a particular candidate agent. For example, the ratio of expression levels of one or more immune cell gene signatures in the Teff gene set (e.g., one or more of CD8A, GZMA, GZMB, IFNγ, EOMES, or PRF1) are compared to the expression levels of one or more immune cell gene signatures in any of the Treg gene set (e.g., FOXP3), an IB APC gene signature set (e.g., one or more of CD276, PDL1, PDL2, or IDO1), and/or an IB T cell gene signature set (e.g., one or more of CTLA4, BTLA, LAG3, HAVCR2, or PDCD1) to determine whether the patient should be treated with an immunotherapy or would have a likelihood of benefiting from particular immunotherapy.

In other embodiments, the methods include determining the ratio of the presence of the immune cell subtype in a PDT-immune cell construct (e.g., Teff to Treg, Teff to B cells, Teff to NK cells, Teff to IB T cell, Teff to IB APC, Teff to inflammatory cells) in a sample from a patient with cancer (e.g., bladder cancer, breast cancer, colorectal cancer, gastric cancer, liver cancer, melanoma, lung cancer, ovarian cancer, or renal cell carcinoma).

The expression level of a cell gene signature in a PDT-immune cell construct are assessed by any method known in the art suitable for determination of specific protein levels in a patient sample. Such methods are well known and routinely implemented in the art, and corresponding commercial kits that are readily available. Preferably, the expression levels of the marker/indicator proteins of the invention are assessed using the reagents and/or protocol recommendations of the kit manufacturer. The skilled person will also be aware of further means for determining the expression level of a cell gene signature. Therefore, the expression level of one or more of the markers/indicators of the invention is routinely and reproducibly determined by a person skilled in the art without undue burden. However, to ensure accurate and reproducible results, the invention also encompasses the testing of patient samples in a specialized laboratory that can ensure the validation of testing procedures.

In some embodiments, the expression level of a cell gene signature is assessed in a PDT-immune cell construct that contains or is suspected to contain cancer cells. In aspects of the invention comprising, consisting of, or consisting essentially of the determination of gene expression in the PDT-immune cell construct, wherein the sample comprises both cancer/tumor cells and non-cancerous cells that are, e.g., associated with the cancer/tumor cells as well as immune cells. In some embodiments, the sample obtained from the patient is collected prior to beginning any treatment regimen or therapy, e.g., chemotherapy or radiation therapy for the treatment of cancer or the management or amelioration of a symptom thereof. In some embodiments, the sample obtained from the patient is collected after beginning a treatment regimen or therapy, e.g., chemotherapy or radiation therapy for the treatment of cancer or the management or amelioration of a symptom thereof.

In some embodiments, the expression level of one or more of an immune cell gene signature can also be determined on the protein level in the PDT-immune cell construct. In some embodiments, the expression level of the marker/indicator proteins are reflected in increased or decreased expression of the corresponding gene(s) encoding the gene signature in the PDT-immune cell construct. Therefore, a quantitative assessment of the gene product prior to translation (e.g., spliced, unspliced or partially spliced mRNA) is performed on the PDT-immune cell construct in order to evaluate the expression of the corresponding gene(s). The person skilled in the art is aware of standard methods to be used in this context or may deduce these methods from standard textbooks. For example, quantitative data on the respective concentration/amounts of mRNA encoding one or more of an immune cell gene signature as described herein is obtained by Northern Blot, Real Time PCR, and the like.

### Methods of Treating a Patient

Disclosed is a method of treating a human patient having cancer, the method comprising, consisting of, or consisting essentially of: (i) determining the expression level of a gene or protein in a PDT as disclosed herein derived from the patient wherein an increase in the level of expression of a gene or protein relative to a median level identifies the patient for treatment with a candidate agent; and (ii) administering the candidate agent to the patient.

Disclosed are methods for administering an activating or suppressing a therapy to patients with a cancer (e.g., bladder cancer, breast cancer, colorectal cancer, gastric cancer, liver cancer, melanoma, lung cancer, ovarian cancer, or renal cell carcinoma that is chemotherapy-resistant, chemotherapy-sensitive, refractory, primary, advanced, or recurrent), if the patient is determined to have a change in the level of expression of one or more gene signatures in their PDT as disclosed herein. In one embodiment, the patient is administered an activating immunotherapy if there is an increase in expression level of one or more immune cell gene signatures in the Teff gene set (i.e., one or more of CD8A, GZMA, GZMB, IFNγ, EOMES, or PRF1) in the PDT or a decrease in expression level of one or more immune cell gene signatures in the Treg gene set in the PDT. In other embodiments, the patient is administered a suppressing immunotherapy if there is an increase in expression level of one or more immune cell gene signatures in the Treg gene set (i.e., FOXP3) in the PDT-immune cell construct or a decrease in expression level of one or more immune cell gene signatures in the Teff gene set (i.e., one or more of CD8A, GZMA, GZMB, IFNγ, EOMES, or PRF1) in the PDT.

In some embodiments, a fixed dose of the candidate agent is administered. The fixed dose may suitably be administered to the patient at one time or over a series of treatments. Where a fixed dose is administered, preferably it is in the range from about 20 mg to about 2000 mg. In one embodiment, one or more loading dose(s) of the candidate agent are administered, followed by one or more maintenance dose(s). In another embodiment, a plurality of the same dose is administered to the patient. In some embodiments, the patient is treated with a combination of candidate agents, and one or more (additional) chemotherapeutic agent(s).

### Cancer Types

The methods of the disclosure are applicable to any cancer. In some embodiments, the cancer are one or more of adenoma, adenomatous polyps, renal carcinoma, adrenal adenoma, thyroid adenoma, pituitary adenoma, parathyroid adenoma, hepatocellular adenoma, fibroadenoma, cystadenoma, bronchial adenoma, sebaceous adenoma, prostate adenoma, adenocarcinoma, cholangiocarcinoma, squamous cell cancer, ductal carcinoma, lobular carcinoma, carcinoma, adenosquamous carcinoma, anaplastic carcinoma, large cell carcinoma, small cell carcinoma, spindle cell carcinoma, sarcomatoid carcinoma, pleomorphic carcinoma, carcinosarcoma, basal cell carcinoma, VIPoma, linitis plastic, adenoid cystic carcinoma, renal cell carcinoma, mucoepidermoid carcinoma, bowel cancer, cancer of the small intestine, colon cancer, colorectal cancer, gastrointestinal cancer, oesophageal cancer, rectal cancer, vaginal cancer, pancreatic cancer, stomach cancer, ovarian cancer, cervical cancer, endometrial cancer, small cell lung carcinoma, non-small lung carcinoma, breast cancer and melanoma. Cancers to which methods of the disclosure are particularly applicable include epithelial cancer, such as gastrointestinal cancer or colorectal cancer, pancreatic cancer, and breast cancer.

The disclosure is applicable to cancer at any stage of progression. Cancer progressions are characterized in several systems. The TNM (Tumor, Node, Metastasis) system comprises three categories, each assigned a numerical degree. T refers to the size of the cancer and how far it has spread into nearby tissue—it is 1, 2, 3 or 4, with 1 being small and 4 large. N refers to whether the cancer has spread to the lymph nodes—it is between 0 (no lymph nodes containing cancer cells) and 3 (lots of lymph nodes containing cancer cells). M refers to whether the cancer has spread to another part of the body—it can either be 0 (the cancer hasn't spread) or 1 (the cancer has spread). A second system is the Numerical Staging System, which comprises four stages. Stage 1 usually means that a cancer is relatively small and contained within the organ it started in. Stage 2 usually means the cancer has not started to spread into surrounding tissue, but the tumor is larger than in stage 1. Sometimes stage 2 means that cancer cells have spread into lymph nodes close to the tumor. This depends on the particular type of cancer. Stage 3 usually means the cancer is larger. It may have started to spread into surrounding tissues and there are cancer cells in the lymph nodes in the area. Stage 4 means the cancer has spread from where it started to another body organ. This is also called secondary or metastatic cancer. The Grading System is a third system of characterizing the extent of progression of cancer. In grade I, cancer cells that resemble normal cells and aren't growing rapidly. In grade II, cancer cells that don't look like normal cells and are growing faster than normal cells. In Grade III, cancer cells that look abnormal and may grow or spread more aggressively.

Certain candidate agents, such as immunotherapies, are more relevant in later (metastasized) stages of cancers such as colorectal cancers, because often surgical resection is enough when no metastasis is present. Accordingly, the disclosure is applicable to cancer at or below one of Stage III, Grade III, or T2 N1 M1. For other cancers that are less easy to resect surgically, therapy can also be relevant at earlier stages. Further, use of the disclosure enables investigation of treatments for cancers at earlier stages. Accordingly, the disclosure is applicable to cancer at or below one of Stage II, Grade II, or T2 N1 M0.

### Immune Diseases

In addition to cancers, diseases of immune cells are investigated using methods of the disclosure. In principle, any disorder of the immune system that affects immune cells are investigated in co-culture. Preferred immune diseases include immune diseases of the digestive and respiratory systems, especially the intestine and lungs. Exemplary immune diseases include irritable bowel disease (IBD), ulcerative colitis (UC), chronic obstructive pulmonary disease (COPD), and asthma.

### Extracellular Matrix

In some embodiments, the cells are cultured in a microenvironment that mimics at least in part a cellular niche in which said cells naturally reside. A cellular niche is in part determined by the cells and by an extracellular matrix (ECM) that is secreted by the cells in said niche. Cellular niches are mimicked by culturing said cells in the presence of biomaterials or synthetic materials that provide interaction with cellular membrane proteins, such as integrins. An extracellular matrix as described herein is therefore any biomaterial or synthetic material or combination thereof that mimics the in vivo cellular niche, e.g., by interacting with cellular membrane proteins, such as integrins. Any suitable extracellular matrix may be used in the Organoid and tumoroid media and/or tumoroid/organoid immune cell culture media.

In some embodiments, the cells are cultured in contact with an ECM. “In contact” means a physical or mechanical or chemical contact, which means that for separating said resulting organoid/tumoroid or population of epithelial cells from said extracellular matrix a force needs to be used. In some embodiments, the ECM is a three-dimensional matrix. In some embodiment, the cells are embedded in the ECM. In some embodiments, the cells are attached to an ECM. A culture medium of the disclosure is diffused into a three-dimensional ECM.

In another embodiments, the ECM is a suspension, i.e., the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%.

Examples of commercially available extracellular matrices include extracellular matrix proteins (Invitrogen) and basement membrane preparations from Engelbreth-Holm-Swarm (EHS) mouse sarcoma cells (e.g., Cultrex® Basement Membrane Extract (Trevigen, Inc.) or Matrigel™ (BD Biosciences)).

In some embodiments the ECM is a laminin-containing ECM such as Matrigel™ (BD Biosciences). In some embodiments, the ECM is Matrigel™ (BD Biosciences), which comprises laminin, entactin, and collagen IV. In some embodiments, the ECM comprises laminin, entactin, collagen IV and heparin sulphate proteoglycan (e.g., Cultrex® Basement Membrane Extract Type 2 (Trevigen, Inc.)). In some embodiments, the ECM comprises at least one glycoprotein, such as collagen and/or laminin. Mixtures of naturally-produced or synthetic ECM materials are used, if desired. In some embodiments, the ECM is BME (‘basement membrane extract’), which is a soluble form of basement membrane purified from Engelbreth-Holm-Swarm (EHS) tumor (e.g., Cultrex® BME).

In another embodiment, the ECM are a synthetic ECM. For instance, a synthetic ECM, such as ProNectin (Sigma Z378666) are used. In a further example, the ECM are a plastic, e.g., a polyester, or a hydrogel. In some embodiments, a synthetic matrix is coated with biomaterials, e.g., one or more glycoprotein, such as collagen or laminin.

A three-dimensional ECM supports culturing of three-dimensional organoids/tumoroids organoids/tumoroids-immune cells. The extracellular matrix material will normally be a drop on the bottom of the dish in which cells are suspended. Typically, when the matrix solidifies at 37° C., the medium is added and diffuses into the ECM. The cells in the medium stick to the ECM by interaction with its surface structure, for example interaction with integrins.

The culture medium and/or cells are placed on, embedded in, or mixed with the ECM.

Preferred ECM's for culturing tumoroids/organoids and organoids/tumoroids-immune cells include BME and Matrigel.

A preferred ECM for culturing tumoroids/organoids and organoids/tumoroids-immune cells co-cultures is collagen, such as rat tail collagen I. The collagen may constitute at least 5%, at least 6%, at least 7%, at least 8%, at least 9% or at least 10% (v/v) of the tumoroid-immune cell co-culture.

In some embodiments, the tumoroid-immune cell co-culture media does not comprise an interleukin (IL).

In some embodiments, tumoroid-immune cell co-culture medium and/or organoid/tumoroid-immune cell co-culture medium comprises a mixture of (a) the immune cell expansion medium (b) the tumoroid culture medium or organoid culture medium and (c) co-culture medium comprising, consisting of, or consisting essentially of oxygen, optionally wherein the media are present at a 33:33:33 or 50:25:25 or 1:1:98 or 25:25:50 or 25:50:25 or 10:10:80 or 15:15:70 (v/v) ratio.

### Persistence and Activity Duration.

In some embodiments, tumoroid immune cell co-culture media of the disclosure allow immune cells to persist for at least 4 hours, 8 hours, 24 hours, 48 hours, 72 hours, 96 hours, 120 hours, 144 hours, 168 hours, 192 hours, 216 hours, 240 hours or up to ten days. In some embodiments, tumoroid immune cell co-culture media of the disclosure allow tumor cells to persist for at least 4 hours, 8 hours, 24 hours, 48 hours, 72 hours, 96 hours, 120 hours, 144 hours, 168 hours, 192 hours, 216 hours, 240 hours or up to ten days. In some embodiments, tumoroid immune cell co-culture media of the disclosure allow immune cells and tumor cells to persist for at least 4 hours, 8 hours, 24 hours, 48 hours, 72 hours, 96 hours, 120 hours, 144 hours, 168 hours, 192 hours, 216 hours, 240 hours or up to ten days. In some embodiments, the tumoroid-immune cell co-cultures can persist for 10 days or more, or for as many days as the tumoroid-immune cell co-culture can remain in culture without being passaged.

In some embodiments, the tumoroid immune cell co-culture media of the disclosure allow the immune cells to remain active for at least 4 hours, 8 hours, 12 hours, 24 hours, 48 hours, or 72 hours after co-culture formation (i.e., after the point of mixing immune cells with organoid/tumoroid cells). Activity of immune cells is detected according to cellular morphology (e.g., the absence of round shape and presence of cellular projections indicates that the cells remain active).

### Disclaimers

In some embodiments, IL-2 is not used in any medium of the claimed invention.

In some embodiments, the PDT is generated without enzymatic separation.

In some embodiments, the PDT is generated without disintegration into single cells.

In some embodiments, the PDT is generated without stromal components of the patient tumor.

In some embodiments, the PDT is generated without disintegration of the PDT to single cells.

In some embodiments, the PDT is generated with enzymatic separation but without removal of stromal cells. In some embodiments, the PDT is generated with enzymatic separation but without removal of stromal cells or reconstituted stromal cells. In some embodiments, the PDTs and systems disclosed herein are generated by mechanical separation. In some embodiments, the PDTs and systems disclosed herein are generated by maintaining stromal and epithelial components in an intact TME. In some embodiments, the PDTs and systems disclosed herein are generated by mechanical separation, which are grown as small tumor chunks, and grown with stromal and epithelial components maintaining an intact TME.

### Kits

The disclosure provides kits comprising, consisting of, or consisting essentially of any organoid, tumoroid, or co-culture of the disclosure.

In some embodiments, the kit comprises one or more of the following: syringe, alcohol swab, cotton ball, gauze pad, instructions for performing the methods of the disclosure.

The disclosure is understood by the following numbered paragraphs:
1. A method for identifying an agent suitable for treating a cancer, wherein the method comprises:
contacting a tumoroid-immune cell co-culture with one or more candidate agents, wherein the tumoroid-immune cell co-culture comprises at least one immune cell and at least one tumoroid;
detecting the presence or absence of one or more change in the tumoroid-immune cell co-culture that is indicative of candidate agent suitability for treating the cancer, and
identifying a candidate agent as suitable for treating the cancer if the presence or absence of one or more of said changes in the tumoroid-immune cell co-culture is detected.
2. The method of claim 1, wherein a candidate agent is identified as a suitable agent if the presence or absence of a change is detected in the tumoroid-immune cell co-culture but not in a reference organoid/tumoroid or reference tumoroid-immune cell co-culture.
3. The method of claim 1, wherein both the tumoroid-immune cell co-culture and the reference organoid/tumoroid or reference tumoroid-immune cell co-culture are derived from the same patient.
4. The method of claim 1, wherein the tumor cell and/or normal cell is obtained from a sample from a cancer patient and/or the tumor cell and normal cell are obtained from the same cancer patient, optionally from the same sample.
5. The method of claim 1, wherein the at least one tumoroid and/or at least one organoid
(a) comprises or consists of autologous cells and/or
(b) are separated into populations sharing one or more genotypes, phenotypes, and/or epigenetic markers, prior to the mixing with immune cells.
6. The method of claim 1, wherein:
(a) the at least one tumoroid or at least one organoid comprises or consists of mammalian cells, preferably human cells; and/or
(b) the tumoroid-immune cell co-culture persists in the tumoroid-immune cell co-culture medium, or wherein the reference organoid/tumoroid or reference tumoroid-immune cell co-culture persists in the organoid/tumoroid-immune cell co-culture medium, for at least 4 h, 8 h, 24 h, 48 h, 72 h, 96 h, 120 h, 144 h, 168 h, 192 h, 216 h and 240 h.
7. A population of tumoroids or organoids prepared according to the method of claim 1.
8. A tumoroid-immune cell co-culture and/or reference organoid/tumoroid-immune cell co-culture as described in claim 1.
9 A method of testing a CAR-T immunotherapy, TCR transgenic T cells, neoantigen, or checkpoint inhibitor, for efficacy and/or safety when used for treating cancer, the method comprising, consisting of, or consisting essentially of:
optionally providing from the same patient tumor cells, normal cells, and immune cells,
expanding the tumor cells in tumoroid culture medium to form a tumoroid, and culturing the tumoroid with the immune cells in a tumoroid-immune cell co-culture medium comprising, consisting of, or consisting essentially of oxygen to form a tumoroid-immune cell co-culture,
expanding the normal cells in organoid culture medium to form an organoid, and culturing the organoid with the immune cells in an organoid/tumoroid-immune cell co-culture medium comprising, consisting of, or consisting essentially of oxygen to form a reference organoid/tumoroid-immune cell co-culture,
contacting the tumoroid-immune cell co-culture and reference organoid/tumoroid-immune cell co-culture with the CAR-T immunotherapy, TCR transgenic T cells, neoantigen, or checkpoint inhibitor,
detecting the presence or absence of one or more change in the tumoroid-immune cell co-culture and reference organoid/tumoroid-immune cell co-culture, wherein the presence or absence of one or more change is indicative of efficacy and/or safety of the CAR-T immunotherapy, TCR transgenic T cells, neoantigen, or checkpoint inhibitor, and
comparing the tumoroid-immune cell co-culture and reference organoid/tumoroid-immune cell co-culture.
10. A method of testing a candidate compound for efficacy and/or safety when used for treating epithelial cancer, the method comprising, consisting of, or consisting essentially of:
optionally providing from the same patient tumor cells, normal cells, and immune cells,
expanding the tumor cells in tumoroid culture medium to form a tumoroid, and culturing the tumoroid with the immune cells in a tumoroid-immune cell co-culture medium comprising, consisting of, or consisting essentially of interleukin to form a tumoroid-immune cell co-culture,
expanding the normal cells in organoid culture medium to form an organoid, and culturing the organoid with the immune cells in an organoid/tumoroid-immune cell co-culture medium comprising, consisting of, or consisting essentially of interleukin to form a reference organoid/tumoroid-immune cell co-culture,
contacting the tumoroid-immune cell co-culture and reference organoid/tumoroid-immune cell co-culture with the candidate compound,
detecting the presence or absence of one or more change in the tumoroid-immune cell co-culture and reference organoid/tumoroid-immune cell co-culture, wherein the presence or absence of one or more change is indicative of efficacy and/or safety of the candidate compound, and
comparing the tumoroid-immune cell co-culture and reference organoid/tumoroid-immune cell co-culture.
11. A method for preparing a tumoroid-immune cell co-culture, wherein the method comprises:
culturing tumor cells in contact with an extracellular matrix in a tumoroid culture medium to obtain an organoid;
removing said extracellular matrix and tumoroid culture medium from said tumoroid;
culturing an immune cell in an immune cell culture medium;
preparing an immune cell suspension comprising, consisting of, or consisting essentially of immune cells, immune cell culture medium; and
mixing the immune cell suspension comprising, consisting of, or consisting essentially of immune cells with the resuspended tumoroid.
13. A method for testing a therapeutic agent, wherein the method comprises:
contacting an organoid/tumoroid-immune cell co-culture with one or more candidate agents, wherein the organoid/tumoroid-immune cell co-culture comprises at least one immune cell and at least one organoid,
detecting the presence or absence of one or more change in the organoid/tumoroid-immune cell co-culture that is indicative of therapeutic efficacy, and
identifying a candidate agent as a therapeutic agent if the presence or absence of one or more of said changes in the organoid/tumoroid-immune cell co-culture is detected.

### EXAMPLES

### Example 1: Study Using Drug X and COMPASS 1

In Example 1, the effect of the tumoroids disclosed in Compass 1 were assessed following treatment with Drug X and/or Olaparib.

Methods: COMPASS 1: Following the build of 10 Triple Negative Breast cancer (TNBC) tumoroids, drug efficacy in multiple (2-10) models. The responder PDTs were subjected to RNA seq in all groups, followed by Ingenuity pathway analysis to define a set of biomarkers common to the responding PDTs. This was followed by pathway analysis, with confirmation via IHC, IFC, apoptosis and proliferation assays.

Tissue acquisition: tumor pieces were collected from the patient followed by mechanical separation of tumor tissue. COMPASS 1 patient derived tumoroids were built on a matrigel based matrix followed by COMPASSS 1 biobanking. Genomic and proteomic characterization of the tumoroids was then conducted. The COMPASS 1 platform was used to test efficacy of multiple modalities and biomarker expression.

FIGS. 1A and 1B describes testing the most effective treatment length for PARP+Olaparib (PARP inhibitor). Different combination of Olaparib and drug x inhibitor was tested at different concentrations against 10 Triple Negative Breast Cancer (TNBC) COMPASS1 tumoroids. A. Vehicle. B. Drug X (100 nM). C. Drug X (1 μM). D. Drug X (10 μM). E. Olaparib (1 μM). F. Combo 1 (X 100 nM+O1 μM). G. Combo 2 (X1 μM+O1 μM). H. Combo 3 (X 10 μM+O1 μM). I. Combo 4 (X100 nM+O5 μM). J. Combo 5 (X1 μM+O5 μM). K. Combo 6 (X 10 μM+O5 μM). For most combinations 14 days of treatment to be most effective in reducing tumor volume. Data shown was analyzed 14 days post-treatment. The vehicle is tumoroid treated with the solvent in which the drugs are dissolved, such as DMSO (1%).

FIG. 2 describes testing the effect of different drug combinations tumor volume of a single COMPASS 1 TNBC. The effect of a single treatment of Drug X and a variety of combinations of Drug X with other drugs on tumor volume of COMPASS 1 TNBC PDTs was tested. Data was analyzed by imaging analysis of the changes to the tumor volume 14 days post-treatment.

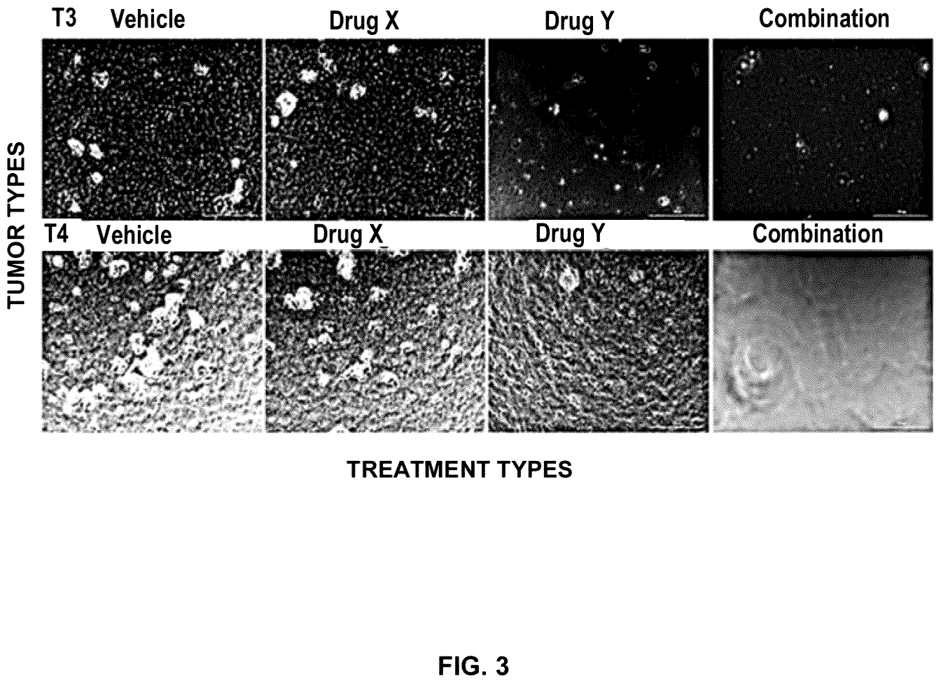

FIG. 3 shows the effect of treatment observed by comparing the tumor volume changes using imaging analysis of 3 biological replicates. This was followed by statistical analysis using t-test for significance (p<0.05) and access vs bliss analysis to test for synergy. Representative images of two tumor types T3 & T4 are shown.

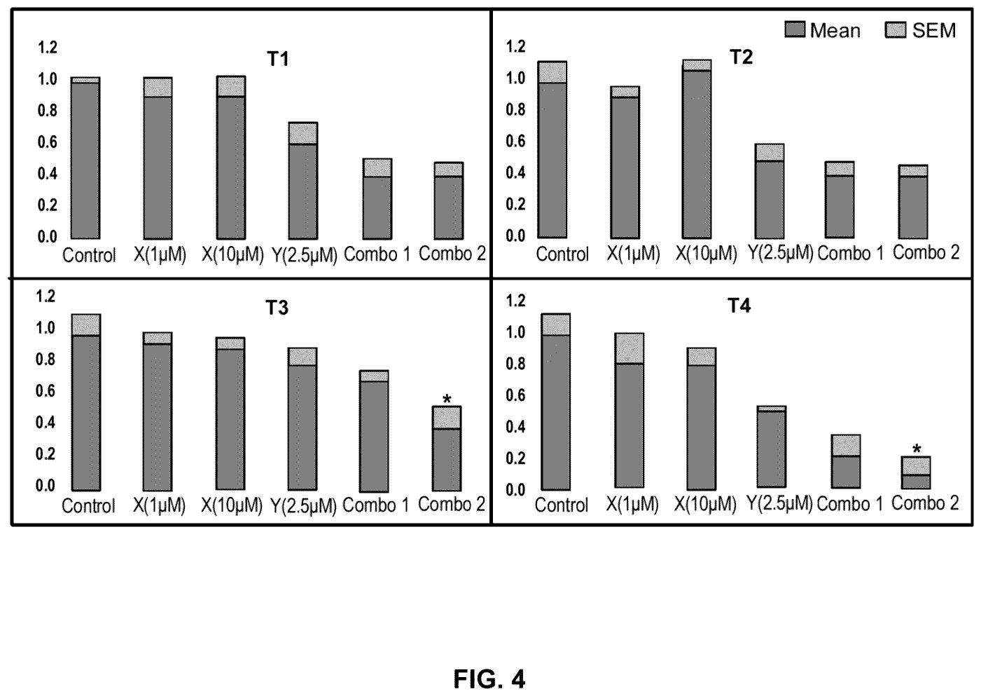

FIG. 4 shows tumor volume reduction post treatment with Drug X, Drug Y, and the combination. Data were analyzed using a t-test to demonstrate a statistically significant decrease in T3 and T4 tumors upon combination treatment.

### Example 2: Study Using Drug Y and COMPASS 1

In Example 2, the effect of the tumoroids disclosed in Compass 1 were assessed following treatment with Drug X, Drug Y and a combination of X and Y where Drug X and Y are different.

FIG. 5 shows the mechanism of cell death upon treatment. The levels of apoptosis were tested in two responders (T3 and T4) and two non-responders (T6 and T7) on Day 7. CellEvent Caspase-3/7 Green assay kit was used to stain the apoptotic cells. The organoids were recovered using cell recovery media and then the cells were stained in each well. Three areas were imaged per well using Biotek Cytation cell imager to get a percentage of apoptosis per well. Each sample was run in three technical replicates for statistically significant results. * p<0.05, n=4.

FIG. 6 shows if individual drug treatment or combination therapy directly affected cell proliferation, the level of proliferation was tested in the PDTs T1-T10. Ki67 was done using FITC tagged Ki67 antibody from Thermo Fisher. Manufacturer recommended protocol for staining was used. Each sample was run in three technical replicates for statistically significant results. * p<0.05, n=4. Representative images of two tumor types T3 & T4.

FIGS. 7A and 7B show RNA Seq analysis of fold changes in significant genes comparing the effect of treatment with tumor vehicle vs. combination treatment. FIG. 7C shows an ingenuity pathway analysis of genes common to T3 and T4.

### Example 3: Study Using Drug Y and COMPASS 2

This example shows the effect of adding PBMCs to the tumoroid.

Methods: COMPASS 2: TNBC COMPASS 2 tumoroids were treated with a lymphocyte infiltration activator which demonstrated an increase in TIL concentration in a time-dependent manner. Such increase in lymphocytes was characterized by measuring CD4 & CD8 levels which reflected control values comparable to patient samples, as well as significantly increased values amongst the treatment groups. All experiments were conducted with n=3 replicates and data was analyzed by One Way ANOVA followed by student's t-test to check for statistical significance.

Methods: Tissue acquisition of tumor pieces from the patient was followed by mechanical separation of tumor tissue. COMPASS 1 patient derived tumoroids built on a matrigel based matrix, co-cultured with PBMC. Compass 1 biobanking followed by Compass 2 patient derived tumoroids built on matrigel based matrix-co-cultured with PBMC. Genomic and proteomic characterization of the tumoroids was then conducted. The types of immune cells present on COMPASS 2 include lymphocyte (T-cell, B-cell neoplastic), monocyte, natural killer cells, macrophages, basophils, dendric cells (plasmacytoid), neutrophils and combinations thereof. COMPASS 2 was used for testing efficacy of multiple modalities and biomarker expression. COMPASS 2 was used for testing multiple immune based modalities like NK cells for efficacy and biomarker testing (NK-specific activation, checkpoint inhibition; tumor mediated (antibody-mediated intervention for tumor ligand shedding), adoptive transfer of modified cells (Engineered CARs), cytokines (NK cell recruitment), tri-specific NK engagers. Use of CPMPAS 2 for testing multiple immune-based modalities like CAR-T for efficacy and biomarker testing.

FIG. 8: A PDL-1 positive PDT of TNBC was tested in an Immuno-competent PDT. The results showed a statically significant response to treatment with 10 mM anti-PD1 inhibitor in the COMPASS 2. These results demonstrate an immune checkpoint mediated reduction in tumor volume, an effect not observed on the same model without the addition of PBMCs.

FIG. 9: Incorporation of TILs in PDTs. Adding PBMCs post-treatment with Drug X showed an increase in percentage of TILs in the COMPASS 2 platform. This data shows that that the client's Drug X increased TIIL incorporation over time.

FIGS. 10A-D: Tumor infiltration data 1. Fig. shows TIL infiltration into PDTs from two independent experiments of PDTs 4698 (A &B) and 3807 (C & D) derived from two different breast cancer patients. All treatments were for 7 days at 1% DMSO *p<0.01, one-way ANOVA followed by Dunnett's multiple comparison test (n=4 replicates per sample/experiment).

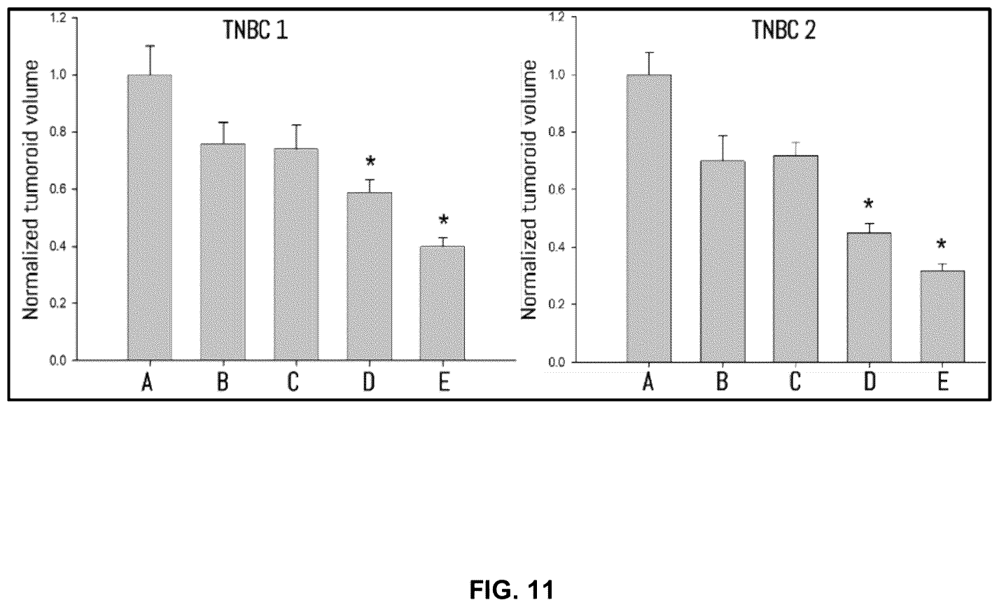

FIG. 11: Using the COMPASS 2 platform, Drug X increased TIIL incorporation over time. Pretreatment of Drug X for 7 days followed by anti-PD1 inhibitor (Pembrolizumab) treatment for 7 days increased TIL infiltration and augmented cell killing significantly in the COMPASS 2 platform. A. Vehicle. B. Vehicle+Pembro (5 μM). C. Vehicle+Pembro (5 μM)+ Drug X (0.3 μM). D. Vehicle+Pembro (5 μM)+ Drug X (1 μM). E. Vehicle+Pembro 5 μM)+ Drug X (3 μM).

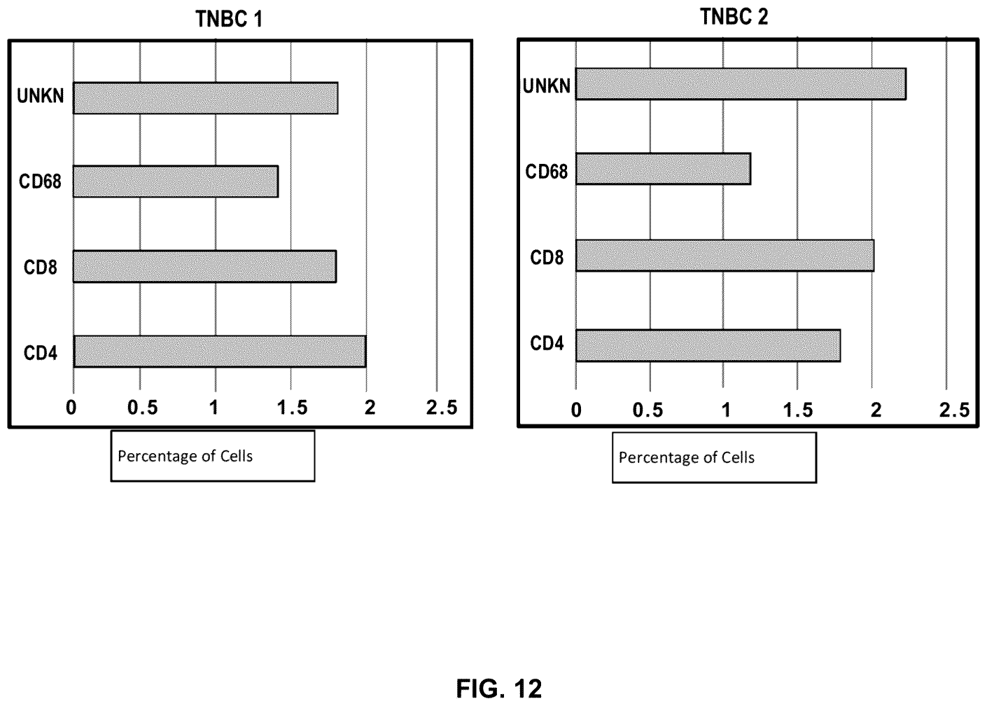

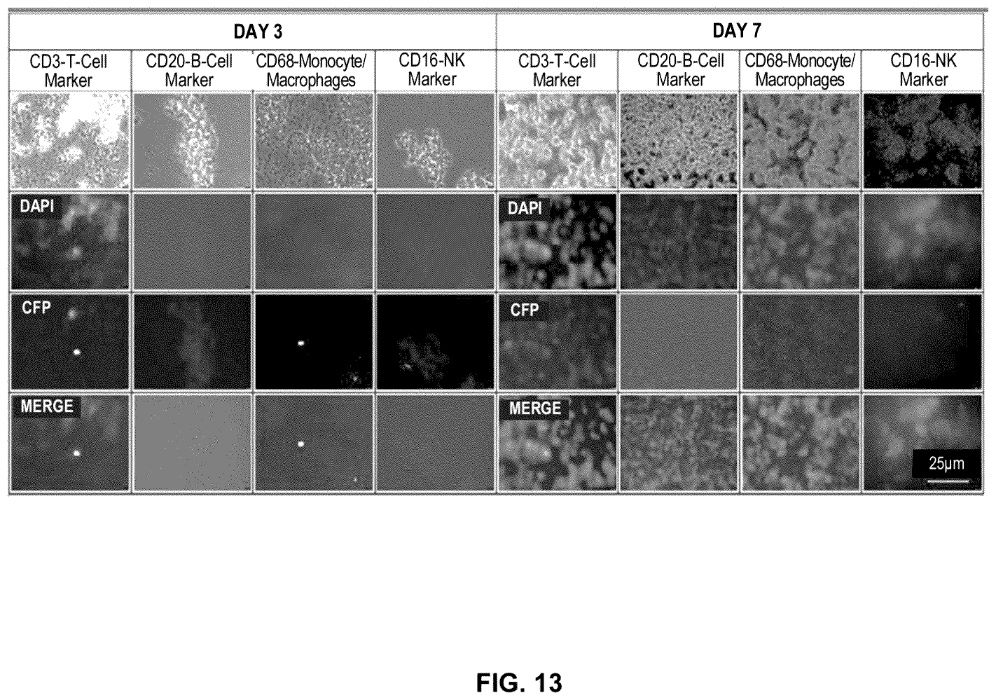

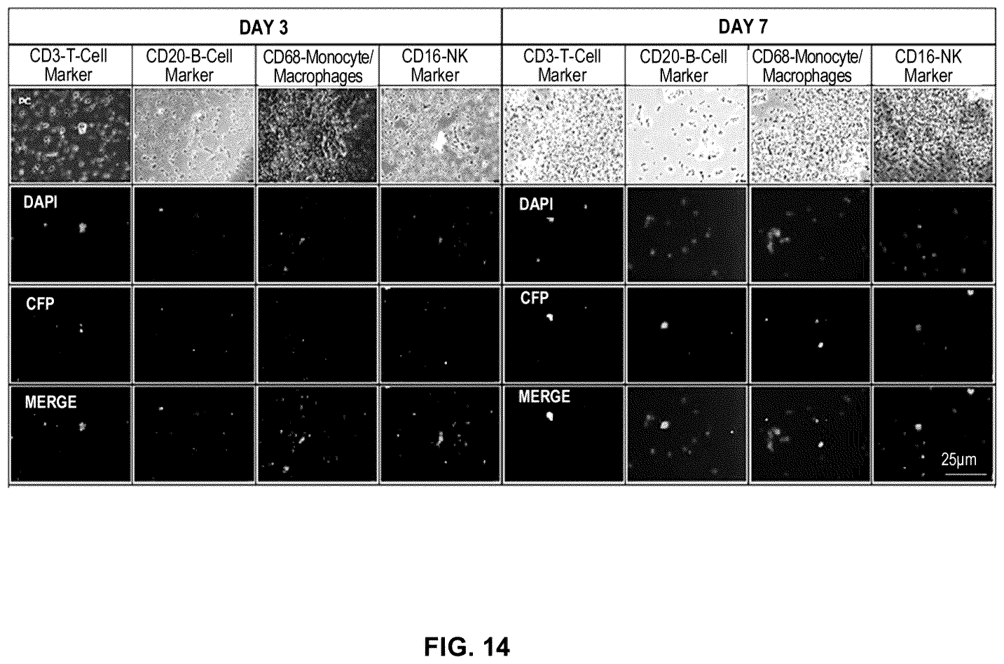

FIG. 12: Tumor infiltration data 2. The Fig shows TIL infiltration into PDTs from two independent experiments of TNBC PDT 1 (left) and 2 (right) derived from two different breast cancer patients. This demonstrates that most of cells are lymphocytes. CD4 (T-helper cells), CD8 (Cytotoxic T-lymphocytes) and C68 (Macrophages). (n=4 replicates per sample/experiment).

### Example 4: Evaluating the Presence and Temporal Distribution of Immune Components in Matrigel-Based Sarcoma and Gallbladder Cancer Patient Derived Tumoroids that have Intact Tumor Microenvironment

This example shows testing for presence of immune components in immune competent Carcinosarcoma tumoroids. Here infiltration depth, extent, and time of CD3+, CD8+, CD4+), B cells (CD20), Macrophages (CD68, CD68+, CD20+ or CD19+, CD16), and NK cells (CD16, CD56+/CD16+) in two different cancer indications to enable multiple forms of therapeutic models are tested on COMPASS 2.

Methods: IMMUNOFLUORESCENCE (IF): PDT Build: High-grade carcinosarcoma and GBC from Spanios biobank. Culture: The COMPASS 1 PDT culture were seeded in 24-well plates, co-cultured with PBMC's (COMPASS 2) and recovered from the Matrigel based matrix. Fixation and Blocking: The PDTs were fixed and washed before blocking for antibody treatment. Blocking was done using an antibody dilution buffer. Testing with antibodies: Primary antibodies for CD3, CD20, CD16, and CD68 were used, followed by secondary antibody and DAPI staining. Imaging: After washing the PDTs were mounted on the slide and imaged using a Leica scope. PBMCs: Prior to evaluating them over time, the PBMCs for the four immune markers were tested on Day 1 of growth using immunocytochemistry and imaged using Leica fluorescent scope.

FACS ACTIVATED CELL SORTING (FACS): PDT Build: High-grade GBC1 and 2 from Spanios biobank. Culture: The COMPASS 1 PDT culture of GBC were seeded in 24-well plates together with PBMC's to form COMPASS 2. Harvest: COMPASS 2 were then isolated from the matrix using a specialized cell recovery solution. Primary antibodies for CD3, CD20, CD16, and CD68 was used. Cell Fixation, Washing and Blocking: Cells were incubated for 15 mins, fixed with 4% PFA for 20 minutes, washed one time and resuspended in FACS buffer blocked with an antibody dilution buffer for cell staining. Cell Staining: For cell surface staining, cells were stained with various markers pre-conjugated with Fluorophore for 15 mins, washed with PBS and then resuspended in PBS containing 1:200 dilution of LIVE/DEAD (7AAD) stain. Imaging: After washing the PDTs were mounted on the slide and imaged. All the samples were analyzed using Beckman Coulter Gallios™ Flow Cytometer.

FIG. 13: Testing for presence of immune components in immune competent Carcinosarcoma tumoroids on Days 3 and 7. Carcinosarcoma were cultured from a tumoroid Biorepository and after the third passage, the tumoroids were recovered, harvested, fixed, and washed before blocking for antibody treatment. Primary antibody was for CD3, CD20, CD16 & CD68 followed by secondary antibody and DAPI staining. The images were taken using Leica inverted fluorescent scope (40×) on Day 7.

FIG. 14: Testing for presence of immune components in immune competent Gall Bladder Carcinoma tumoroids on Days 3 and 7. Gall Bladder Carcinoma were cultured from a tumoroid Biorepository and the tumoroids were recovered, harvested, fixed, and washed before blocking them for antibody treatment. Primary antibody was for CD3, CD20, CD16 & CD68 followed by secondary antibody and DAPI staining. Images were taken using Leica inverted fluorescent scope (40×) on Days 3 and 7.

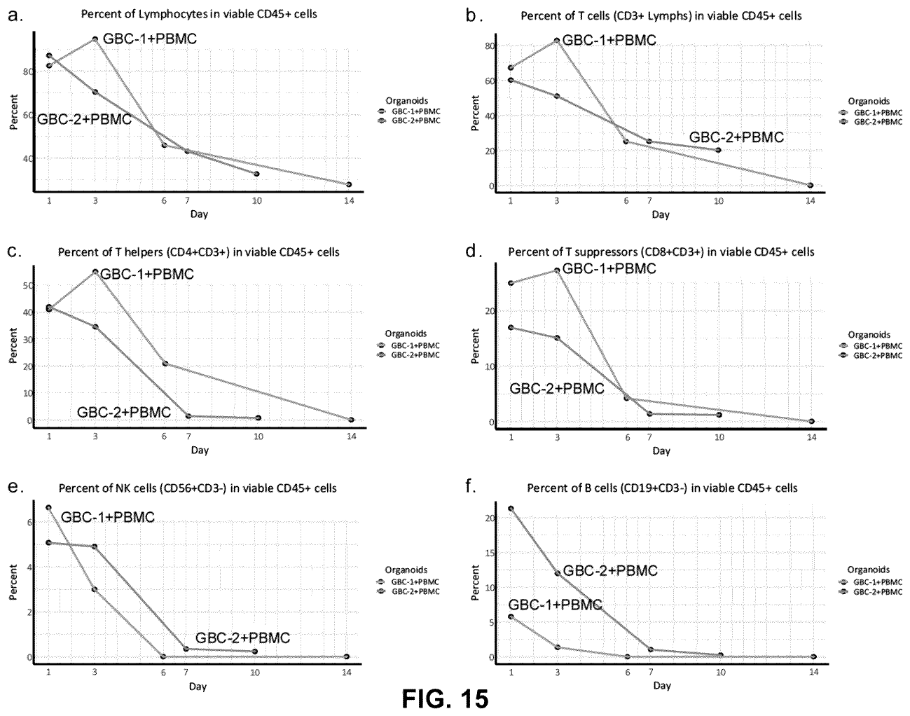

FIG. 15: Percent of a. Lymphocytes, b. T cells (CD3+ Lymphs), c. T helper cells, (CD4+CD3+) and d. T suppressor cells (CD8+CD3+), e. NK cells (CD56+CD3−) and f. cells (CD19+CD3−) in CD45+ cells. This data was graphed to quantitatively show in two different COMPASS 2 models, GBC-1 and GBC-2, the presence of viable lymphocytes (CD45+) and its subsets such as T cells (CD3+), T-helper cells (CD4+) and cytotoxic T cells (CD8+). Additionally, it was demonstrated that the presence of CD56+NK cells and CD19+B cells in T cell negative populations

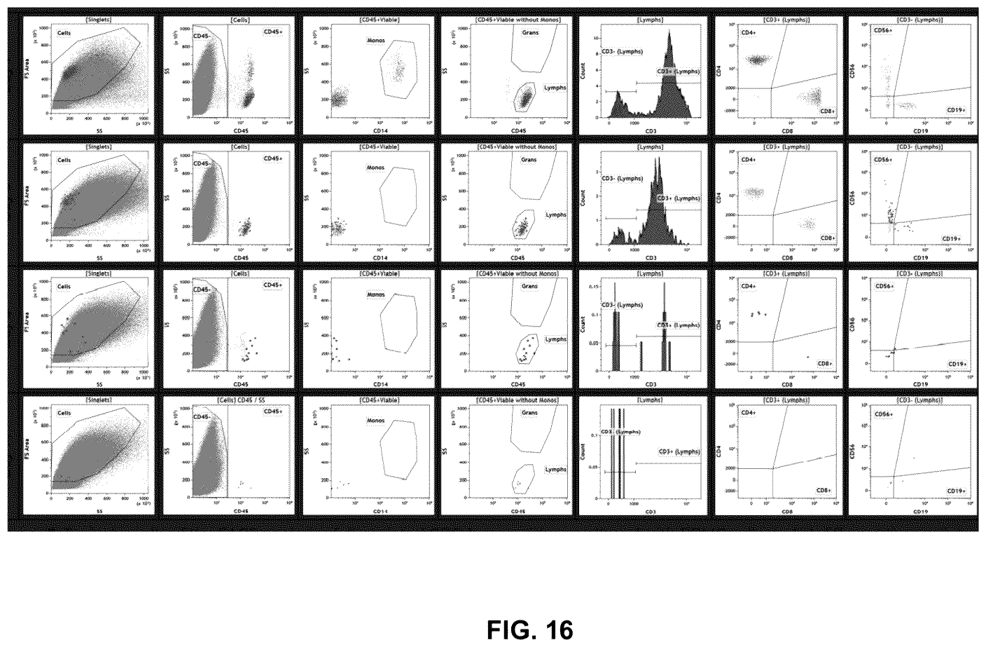

FIG. 16: FACS-analysis of dissociated GBC-1 organoids co-cultured with PBMC collected at different time points: To characterize the immune cells in a semiquantitative manner a comprehensive FACS analysis pattern of the COMPASS 2 tumoroid GBC-1 was conducted. The tumoroids and the immune cells were disassociated and co-cultured and collected at different time points for immune analysis. The cells were defined from left to right on different days (Days 1, 3, 6, and 14) for (i) live/dead cells, (ii) CD45+ (immune cells) and CD45-cells, (iii) Lymphocytes and Monocytes, (iv) Granulocytes.

FIGS. 17A-D: Quantitative live-dead assay: Quantitative analysis of live-dead lymphocytes and tumor cells was performed at different points using 7-AAD, CD45, and counting beads to determine the viability of CD 45+ cells over time (a, b, c, d) in GBC-1. The side panel shows viable tumoroids and viable CD45+ lymphocytes over time to demonstrate that the COMPASS 2 tumoroids grow along with lymphocytes in culture.

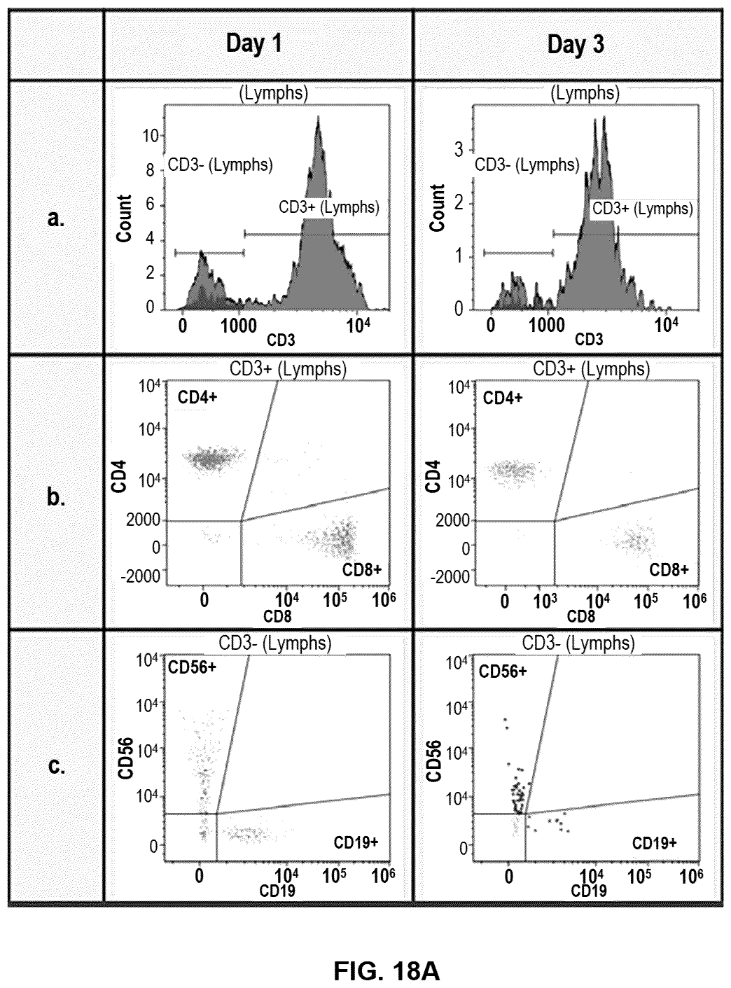

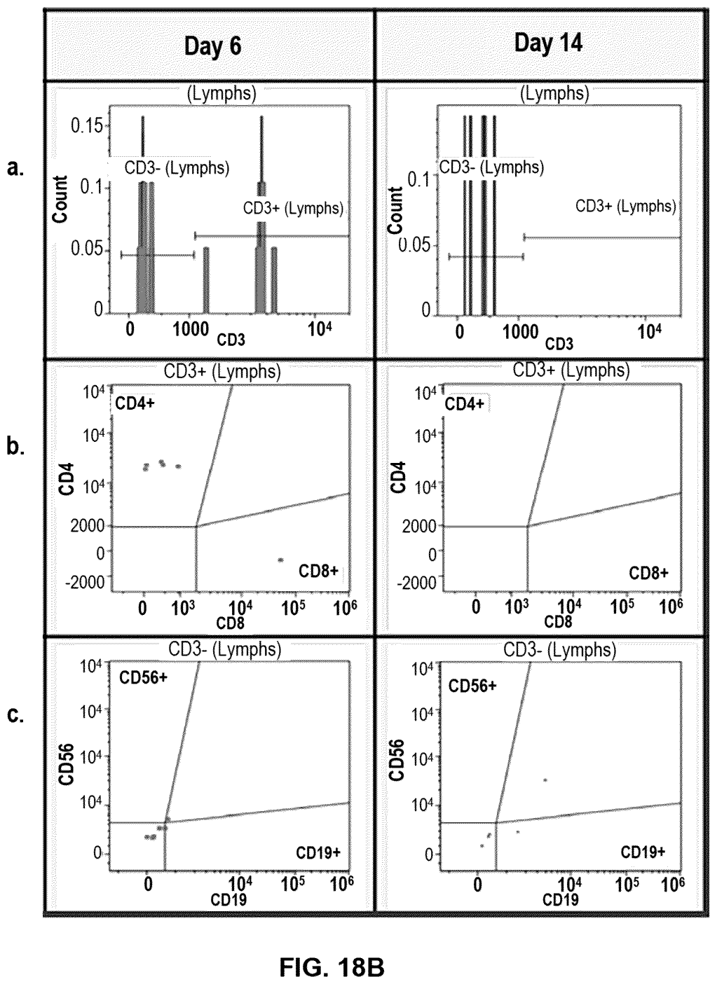

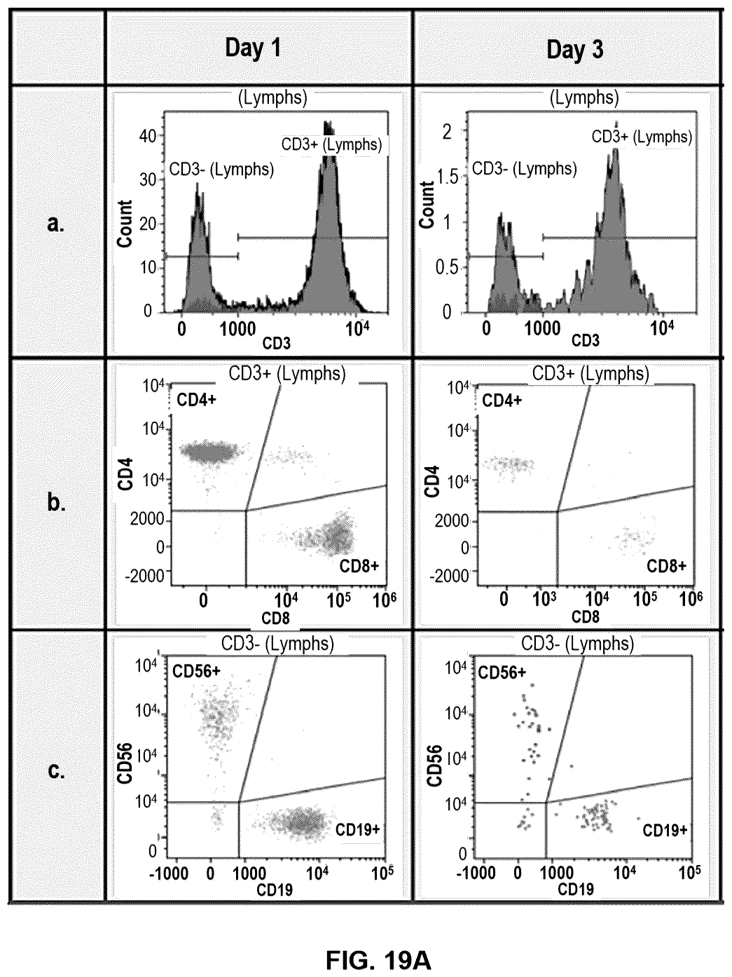

FIGS. 18A-B: Illustrative FACS Analysis of Lymphocyte and Lymphocyte Subset in GBC-1: FACS data analysis was examined in GBC-1 tumoroids co-cultured with PBMCs (Compass 2 model) for lymphocyte (CD45) and lymphocyte subsets, along with other immune cells were collected at different time points of tumoroid growth. Data demonstrated the presence of Lymphocytes (CD45+), T cells (CD3+) their subsets T helper cells (CD4+CD3+) and T suppressor cells (CD8+CD3+) in semi-quantitative manner on Days 1, 3, 6 and 14.

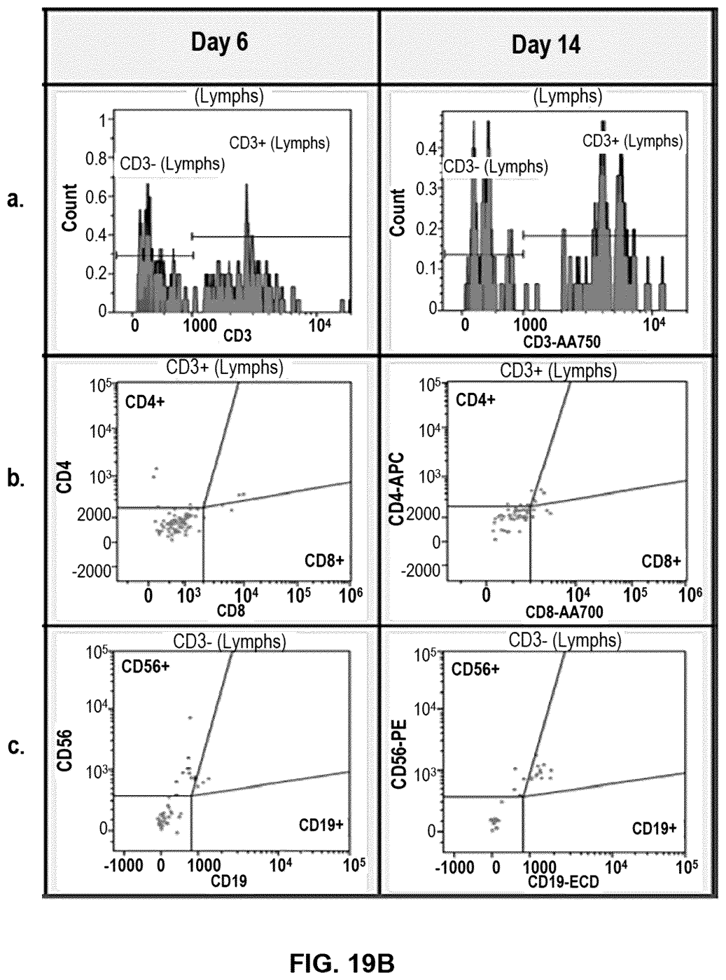

FIGS. 19A-B: Illustrative FACS Analysis of Lymphocyte and Lymphocyte Subset in GBC-2: FACS data analysis was repeated in GBC-2 tumoroids co-cultured with PBMCs (Compass 2 model) for lymphocyte (CD45) and its subsets, which along with other immune cells were collected at different time points of tumoroid growth. Data demonstrated the presence of Lymphocytes (CD45+), T cells (CD3+) their subsets T helper cells (CD4+CD3+) and T suppressor cells (CD8+CD3+) in semi-quantitative manner on Days 1, 3, 6 and 14.

### Example 5: Indication Agnostic Evaluation of T-Cell Function Using Patient Derived Immunocompetent Tumoroid Modelling Platform

This example shows proof of concept data for organoids co-cultured with PBMCs.

Durable responses to immunotherapies in cancer patients are limited due to tumor heterogeneity. Preclinical models are crucial for understanding resistance mechanisms and improving treatment effectiveness. Chimeric antigen receptor (CAR-T) cell therapy shows promise but lacks optimized preclinical models for assessing function, tumor killing, cytokine production, and memory responses. T regulatory cells (Tregs), a subset of CD25+CD4+ cells, regulate self-reactive cells but also hold potential for treating autoimmune and transplant-related disorders. However, the comparative advantages of Tregs and Cytotoxic T lymphocytes (CTLs) in various disease contexts remain poorly understood.

In this study the efficiency of the Patient Derived Tumoroids model platform was evaluated on the Custom Organoid Modelling Platform for Accurate and Speedy Solutions (COMPASS) 2 model by examining the developmental and functional phases of CAR-T cells, Tregs, and CTLs, encompassing activation, memory, and exhaustion. Qualitative and quantitative analyses of surface markers to recapitulate the complexity of human cancer was performed. Following the build and growth of GBC and TNBC on a Matrigel-based matrix using a proprietary medium. Gall Bladder Cancer (GBC) was characterized using Fluorescence Activated Cell Sorting (FACS) analysis with specific antibody markers while Triple Negative Breast Cancer (TNBC) characteristics were analyzed using H&E staining and treated with EGFRvIII-targeted CAR-T cells.

GBC-1 tumoroids co-cultured with PBMCs showed Treg and CTL expression until Day 3. Efficacy studies revealed a significant TNBC-1 tumor volume decrease with EGFRvIII-targeted CAR-T cell treatment. These results validate COMPASS 2 for functional testing of CAR-T cells, Tregs, and CTLs, with observed T cell infiltration impacting the Tumor Microenvironment (TME) Gall bladder Cancer (GBC): GBC is the predominant malignancy within the biliary tract, accounting for 90% of biliarytract cancers. Timely detection is crucial as GBC often progresses silently, leading to fatal outcomes upon late diagnosis. Tregs, a subset of CD25+CD4+ T cells, possess inhibitory properties on antigen-specific T cell activation, impacting immune responses in cancer. CTLs are vital for antitumor immunity, directly targeting and eliminating malignant cells. Both Tregs and CTLs serve as potential diagnostic markers for GBC, regulating harmful immune responses and the body's immunity to foreign antigens. In this study, immune infiltration, immunosuppressive mechanisms, and different immune cell functionalities, including activation, memory, and exhaustion are assessed.

Triple-Negative Breast Cancer (TNBC): TNBC is an aggressive subtype of breast cancer, comprising about 15% of newly diagnosed cases. With chemotherapy as a primary treatment, challengestotreatmentincludecomplexityandlackoftargetedtherapies. TNBC often recurs at high rates and carries a poor prognosis, driving the search for innovative treatment approaches. CAR-T cell-based immune therapy shows promise in harnessing the immune system to target specific antigens in TNBC. The development and optimization of CAR-T cell therapies requires a rigorous preclinical model for the evaluation of CAR-T cell function, tumor killing, cytokine production, and memory responses. Here, the comprehensiveness of the COMPASS 2 as a wide-ranging testing platform for studying CAR-T, Tregs, and CTLs is evaluated.

Study Focus: Evaluate COMPASS 2 model platform as a comprehensive tool to study developmental and functional phases of CAR-T, Tregs, and CTLs cells and understand immune component infiltration and mechanisms of immunomodulation.

Methods: Treg/CTLs Experimental Workflow: Passage 3 GBC Organoids and PMBC were recovered from liquid nitrogen. The cells were thawed in a 37° C. water bath and transferred to a 15 ml tube for washing. Cells were centrifuged for 5 min at 300 g, supernatant was aspirated and resuspended again in the medium. ⋅Cell count was performed followed by co-culturing the GBC ⋅tumoroids and PBMCs in the ratio of 1:0.25 and plated in 24 well plates. FACS analysis was performed to examine the expression levels of Treg and CTLs at Days 1, 3, 5 using Novacyte 3000instrument. Data of n=3 replicates was statistically analyzed using One way ANOVA followed by Tukey test for pairwise comparison.

CAR-T Experimental Workflow: Passage 3 TNBC tumoroids were recovered from liquid nitrogen. The cells were thawed in a 37° C. water bath and transferred to a 15 ml tube for washing. Cells were centrifuged for 5 min at 300 g, supernatant was aspirated and resuspended again in the medium. Cell count was performed, and ˜1000 cells (tumoroids) were plated in 96 well plate on a matrix. EGFR, its T cells mock, and control were added to the COMPASS 2 TNBC tumoroids after 24-72 hrs in the ratio of 1:1, 1:5 and 1:10. Image analysis was performed to observe the tumor volume at 24 hrs, and Days 4 and 8 using Cytation 5. Data of n=3 replicates was statistically analyzed using One way ANOVA followed by Tukey test for pairwise comparison.

FIGS. 20A-B: Representative dot plots of the Treg cells phenotyping CD4+CD25+CD127 dim of PBMC and dissociated GBC-1 organoids co-cultured with PBMCs collected at different timepoints. From Left top to right is PBMC analyzing the presence of Lymphocytes. The second quadrant indicates viable T-Cells. The third quadrant highlights the presence of CD4+ and CD25+. The last quadrant shows presence of Tregs.

FIG. 20C: Representative dot plots of the CTLs activation phenotyping CD8+ HLADR+ of dissociated GBC-1 organoids co-cultured with PBMC collected at different time points. Top Left to right is Day 1 analyzing the presence of Lymphocytes CD45+. The second quadrant indicates viable T-CellsCD3+. The Third quadrant highlights the presence of CD4+ and CD8+ T-Cells. The last Quadrant indicates the presence of Cytotoxic T Lymphocytes (CTLs) (CD+ HLADR+)

TABLE 1

| | | GBC-1 + | GBC-1 + | GBC-1 + |
|---|---|---|---|---|
| | | PBMC | PBMC | PBMC |
| | PBMC | (Day 1) | (Day 3) | (Day 5) |
| | % out of | % out of | % out of | % out of |
| Name | Lymphs | Lymphs | Lymphs | Lymphs |
| CD3+ | 67.16 | 60.59 | 10.93 | 9.48 |
| CD4 + CD3+ (hTLs) | 53.56 | 47 | 4.6 | 2.48 |
| CD4 + CD25+ | 6.59 | 14.26 | 0.86 | 0.24 |
| Tregs | 2.14 | 3.38 | 0.07 | 0.07 |
| (CD3 + CD4 + CD25+ | | | | |
| CD127dim | | | | |

Table 1 presents the percentage of various lymphocyte subsets, including T cell (CD3+), The per lymphocytes (CD4+CD3+), and Tregs (CD3+CD4+CD25+CD127dim), and their changes over different time points (Day1, Day3, Day5) in GBC-1+PBMC co-culture. Notably, there are fluctuations observed in the percentages of these subsets overtime, indicating dynamic changes in the immune cell composition within the co culture system. Additionally, PBMC represents the initial lymphocyte subset composition before co-culturing with GBC organoids.

TABLE 2

| | GBC-1 + | GBC-1 + | GBC-1 + |
|---|---|---|---|
| | PBMC | PBMC | PBMC |
| | (Day 1) | (Day 3) | (Day 5) |
| | % out of | % out of | % out of |
| Name | Lymphs | Lymphs | Lymphs |
| CD3+ | 60.59 | 10.93 | 9.48 |
| CD8 + CD3+ (CTLs) | 11.41 | 4.53 | 1.63 |
| Active CTLs | 3.68 | 0.36 | 0 |
| (CD8 + CD3 + HLADR+) | | | |

Table 2 displays the proportions of different lymphocyte subsets, such a s T cells (CD3+), cytotoxic T lymphocytes (CD8+CD3+), and activated CTLs (CD3+CD8+HLADR+), along with their variations across different time points (Day 1, Day 3, Day 5) in GBC-1+PBMC co-culture. Notably, fluctuations in the percentages of these subsets over time suggest dynamic alterations in the immune cell composition within the tumoroid-immune cell co-culture system.

FIG. 21: Modeling immunotherapy with co-culture of TNBC-1 tumoroids and CAR-T Cells: TNBC-1tumoroids that showed high expression of EGFR were treated with EGFRvIII-targeted CAR-T cells in a ratio of 1:1 and 1:5 and compared with their respective controls. The expression levels of EGFRvIII-targeted CAR-T cells in TNBC-1 tumoroids were then quantified by imaging analysis. Data of n=3 replicates was statistically analyzed using One way ANOVA followed by Tukey test for pairwise comparison.

FIG. 22: Modeling immunotherapy with co-culture of TNBC-2 and CAR-T Cells: TNBC-1 tumoroids that showed no expression of EGFR were treated with EGFRvIII-targeted CAR-T cells in a ratio of 1:1 and 1:5 and compared with their respective controls. The expression levels of EGFRvIII-targeted CAR-T cells in TNBC-2 tumoroids were then quantified by imaging analysis. Data of n=3 replicates was statistically analyzed using One way ANOVA followed by Tukey test for pairwise comparison.

FIG. 23: Representative phase contrast images of TNBC-2 PDTs treated with CAR-T-Mock and EGFR (1:10) for Days 1, 2 and 5. Tumor Volume of TNBC-2 Tumoroids was measured over time. Demonstrated is significant reduction in tumor volume at Day 2 compared to CAR-T-mock treated organoids.

### Example 6: Modeling Versatile Three-Dimensional Patient-Derived Tumoroid (PDT) Drug Testing Platforms by Recapitulating Tumor Microenvironment Oxygenation and Immunocompetence

This example shows proof of concept data for organoids grown on a scaffold with a dynamic liquid-liquid interface. This example shows the effect of adding oxygen to the tumoroid.

COMPASS 3: Human Patient-Derived Tumoroid-based (harvested via mechanical digestion to preserve tumor and stromal cells)—Expanded on a TME structural scaffold platform (fiber glass) With versatile TME sub-components: Oxygenation/Perfusion—via the utilization of an in-well perfusion method with the aid of synthetic hemoglobin3; Immunocompetence-via the addition of commercial human peripheral blood mononuclear cells (PBMCs). Human Uterine Adenosarcoma COMPASS 3 sub-model Growth Profiles-4 arm study (3a-3d) testing variable combinations of oxygenation and immunocompetence across timepoints: Day 1, 3, 7, 14, & 28

Methods: Tissue acquisition of tumor pieces from the patient followed by mechanical separation of tumor tissue. COMPASS 3 patient derived tumoroids built on matrigel based matrix, co-cultured with PBMC. Compass 1 biobanking followed by Compass 3 patient derived tumoroids built on matrigel based matrix-co-cultured with PBMC. Genomic and proteomic characterization of the tumoroids. Tyes of COMPASS 3 platforms: 3a, 3b, 3c and 3d (defined below). Use of COMPASS 3 for testing efficacy of multiple modalities and biomarker expression. Use of COMPASS 2 for testing multiple immune based modalities like CAR-T for efficacy and biomarker testing. Use of CPMPASS 3 for testing oxygenation influenced modalities for efficacy and biomarker testing.

Herein, versatile three-dimensional (3D) patient-derived tumoroid (PDT)-based platforms are disclosed that capture TME diversity. Four subversions of the COMPASS 3 scaffold-based model: “3a” (−) Oxygenation [Static], (−) Immunocompetence; “3b” (−) Oxygenation [Static], (+) Immunocompetence; “3c” (+) Oxygenation [Dynamic], (−) Immunocompetence; & “3d” (+) Oxygenation [Dynamic], +) Immunocompetence. While Example X shows non-immunocompetent COMPASS 3 models slower PDT growth in the presence of oxygenation (*** p<0.001), this study adds another paradigm on the effect of immunocompetence in tumoroid growth in parallel to variable oxygenation states. Observations on the static arm consistently portrays an increase in tumor growth for static, nonimmunocompetent models that plateaus at Day 7 (*, p<0.042); however, such growth phenomenon was not observed on its immunocompetent counterpart given its earlier plateau in growth (***, p<0.001). Remarkably, the dynamic arm of the study, on the other hand, projects a lack of growth profile pattern difference between immunocompetence states. Such observations have implications in the role of TME oxygenation conditions alongside tumor-immune interactions, thus highlighting COMPASS 3's versatility for complex and accurate TME modeling. Methods: 3D Tumor Microenvironment Model Platform. COMPASS 3 (TME Structural Scaffold+Biochemical Matrix; COMPASS 3a-b (Static, Apical Oxygenation—Standard Well Plate; COMPASS 3c-d (dynamic, apical+basal/peri-cellular oxygenation via dynamic matrix-liquid-liquid interface using in-well perfusion method with aid of synthetic hemoglobin); tumor microenvironment cells without non-native Immune Cells (COMPASS 3a & 3c) or with non-native immune cells (COMPASS 3b & 3d); Human Uterine Adenosarcoma (Tumoroid—10-Day old)

Experimental Set-Up: Distinct/Separate Representative Scaffolds/Platforms, N=3 per group, for corresponding timepoints (Days 1, 3, 7, 14, & 28); Groups: I) Static vs Dynamic, II) Non-Immunocompetent vs Immunocompetent.

Evaluation: Imaging—Phase contrast imaging by Biotek Cytation 5 (Agilent)—Whole view, 4×, and 20×.

Analysis: based on Phase Contrast 4× Images (5 manually selected regions) with tumoroid selection (50-500 μm in size) for size and estimated volume measurements.

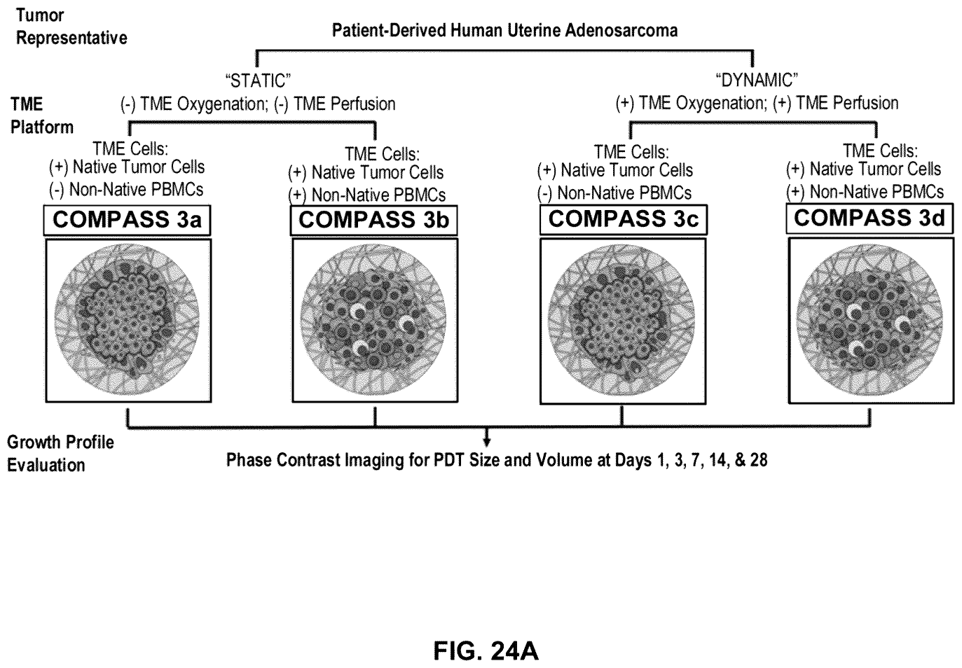

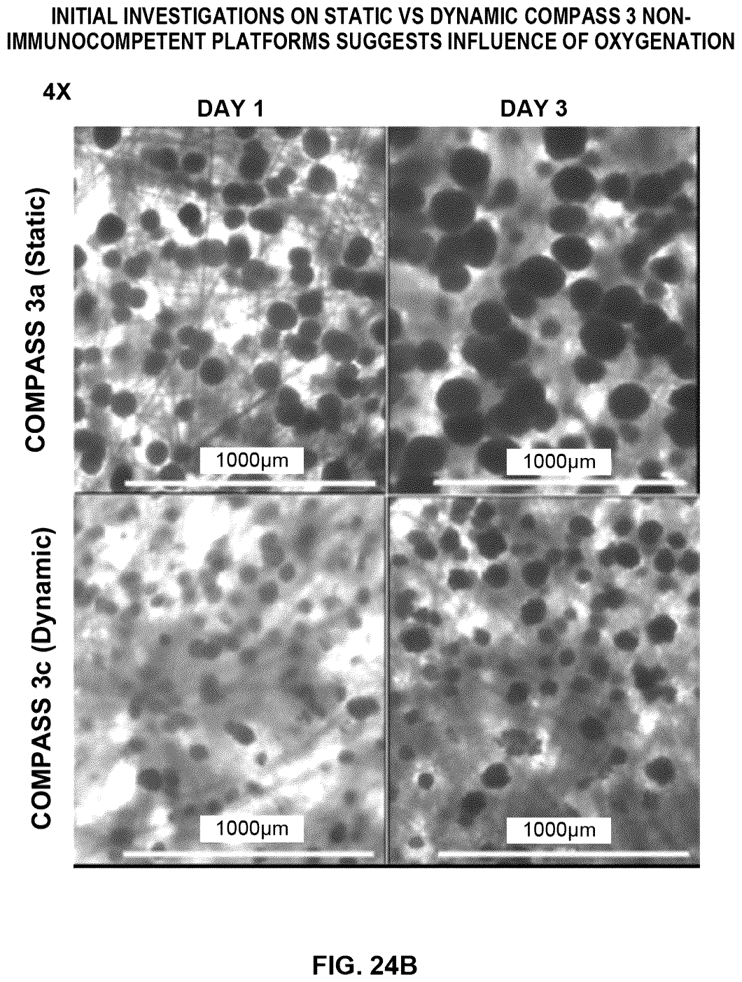

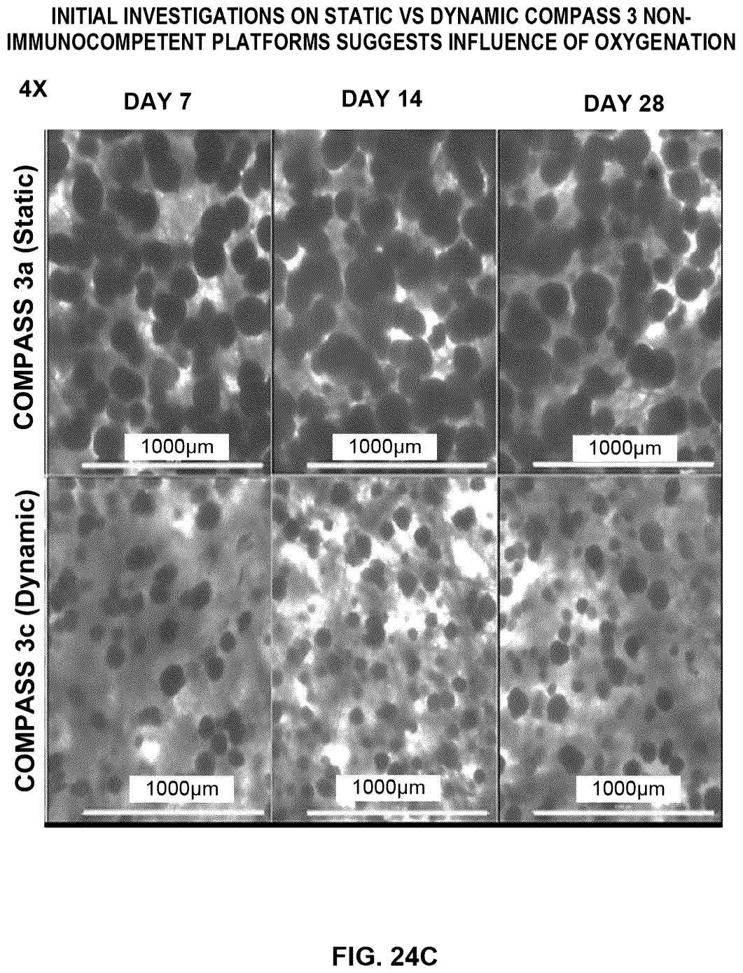

Statistics: Student's T-test amongst non-immune and immune groups per respective timepoints, One-way ANOVA for each sub-arm of the study (per COMPASS 3 sub-version) N=3. (p<0.05). FIG. 24A shows a schematic of the various COMPASS 3 platforms

FIGS. 24B-C: 4× images of static vs dynamic non-immunocompetent COMPASS 3 platform representing various timepoints (day 1, 3, 7, 14, 28) for preliminary growth profile study. Static systems are systems to grow tumors without physiological flow (no perfusion pump). Dynamic systems are systems to grow tumors with physiological flow (having a perfusion pump).

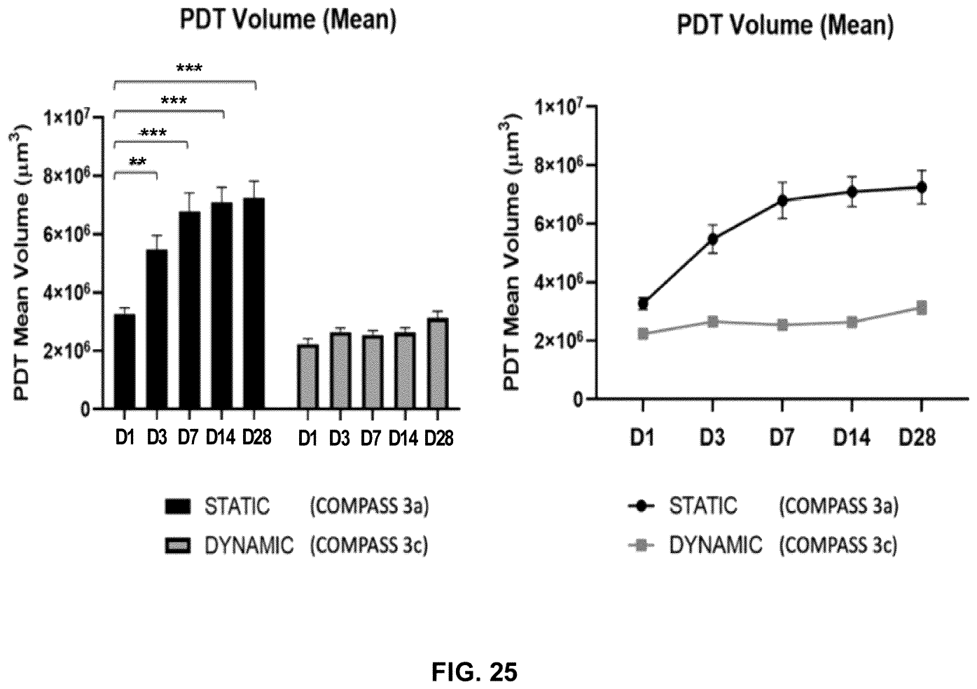

FIG. 25: static vs dynamic non-immunocompetent COMPASS 3 platform mean volume growth profiles. Significant difference between static and dynamic on post day 3, N=3, *** p<0.001. Non-immunocompetent COMPASS 3 models showed slower PDT growth in the presence of oxygenation.

FIG. 26: Whole view of static vs dynamic immunocompetent COMPASS 3 platform on day 3 pre-treatment. N=3; Size: *, p<0.02; Volume: *, p<0.019.

FIGS. 27A-F. Human uterine adenosarcoma growth profiles across variable COMPASS 3 Platforms (static vs dynamic) under variable oxygenation-without and with immunocompetence at different timepoints (Day 1, 3, 7, 14, & 28). Static-Compass 3a&b (left) and Dynamic-COMPASS 3b&d (right) images (A, B, & C) and corresponding estimated mean volume measurements: by day (D) and by group (E). COMPASS platform whole view Images (A). 4× images (B) suggestive of differences of tumor volume difference (E), and 20× images (B) representative of averages sizes. T-test between non-immune and immune groups per respective timepoints. N=3. General difference in growth profiles between non-immunocompetent and immunocompetent static models, especially starting on Day 7. (***), p<0.001. PDT=Patient-Derived Tumoroid. As discussed, static systems have air dissolved oxygen and dynamic systems have perfused oxygen from the synthetic hemoglobin to the media.

COMPASS 3: Soft tissue sarcoma PDTs in a scaffold-based platform (COMPASS 3) were built in the presence and absence of immune components; as well as the presence (dynamic) and absence (static) of supplemental oxygenation. The role of a HSP90 inhibitor in the presence and absence of immune components and oxygenation was investigated to demonstrate the impact of TME on drug treatment. The role of PBMCs and oxygenation was determined, and growth tested over the long term (Day 28) All experiments were conducted with n=3 replicates and data was analyzed by One Way ANOVA followed by student's t-test to check for statistical significance.

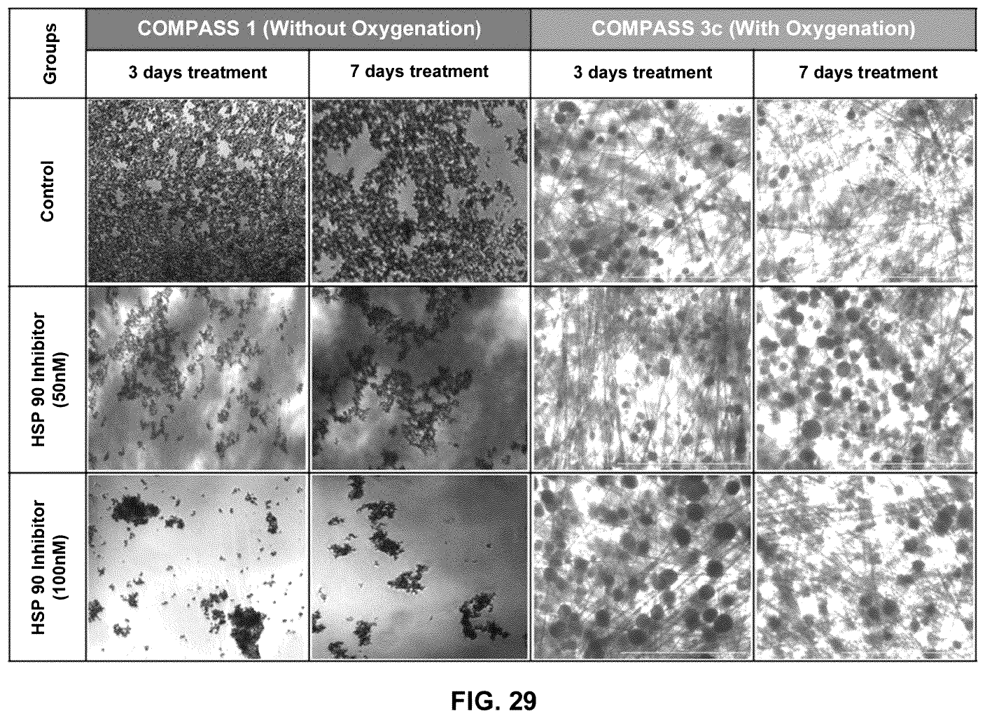

FIG. 29: Sarcoma PDTs were treated with HSP90 Inhibitor at two different concentrations (50 mM and 100 nM) with and without oxygenation on COMPASS 2 and COMPASS 3 models to test the impact of oxygen on tumor volume. The oxygenation was ensured by growing the PDTs on the perfusion system and using synthetic hemoglobin for cellular oxygenation.

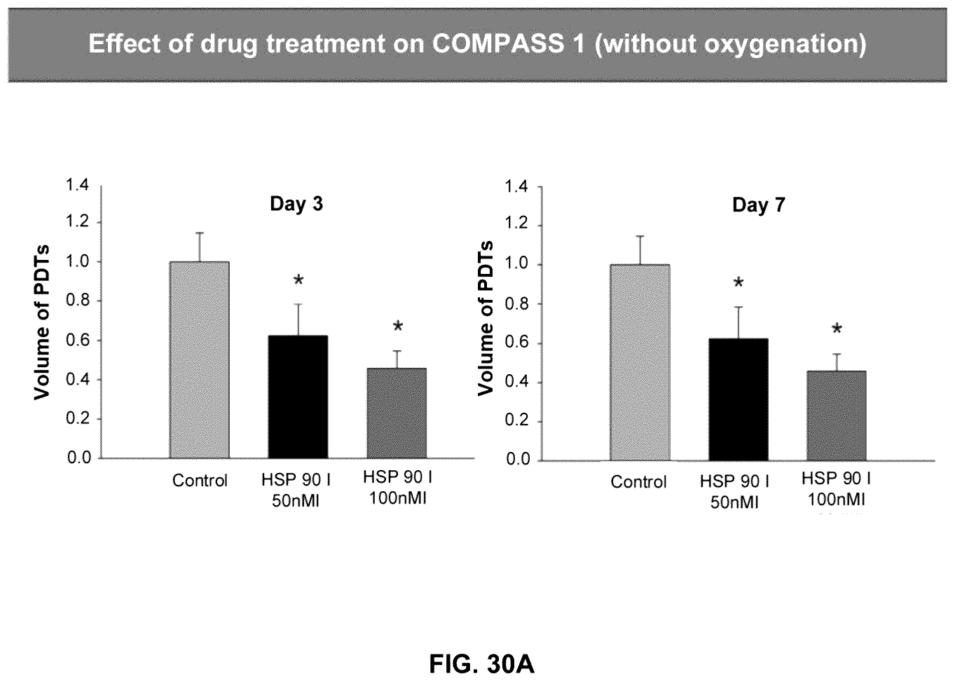

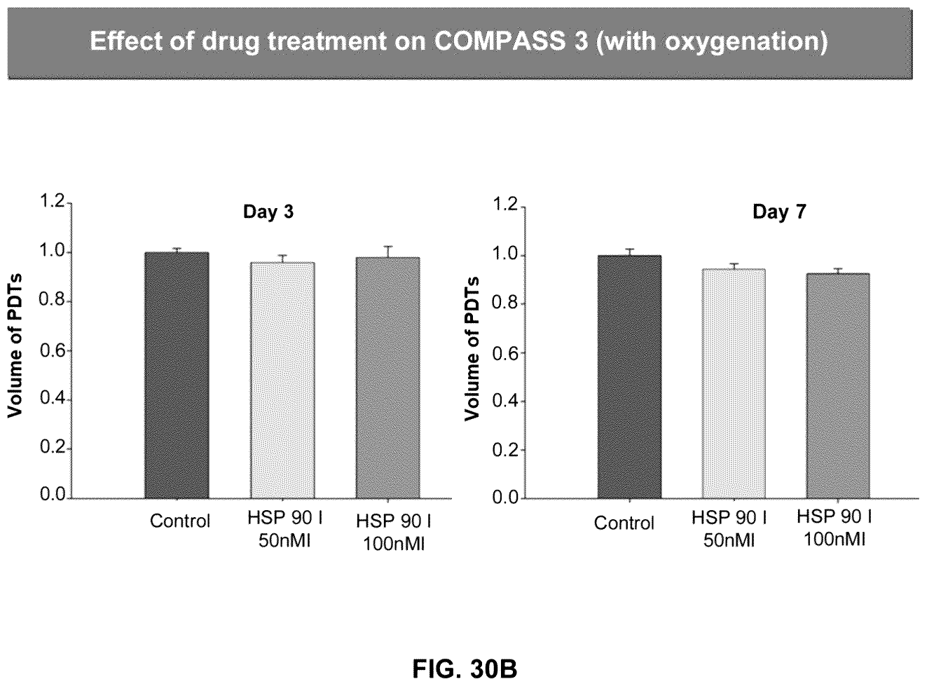

FIG. 30A: Matrigel-based Sarcoma COMPASS 2 were established and treated with two different concentrations (50 nM and 100 nM) of HSP 90 Inhibitor for 3 days and 7 days. The effect of treatment was measured by change in tumor volume. Statistical analysis was done using t-test for significance (p<0.05), n=3. A similar rate of cell growth in the untreated samples was observed in both model systems and HSP90 Inhibitor was effective in reducing tumor volume.

FIG. 30B: Matrigel-based Sarcoma PDTs were established on a Liquid-Liquid Perfusion Interface (LLPI) using a synthetic hemoglobin as a blood substitute. The PDTs were treated with two different concentrations (50 nM and 100 nM) of HSP 90 Inhibitor for 3 days and 7 days. The effect of treatment was measured by change in tumor volume. Statistical analysis was done using t-test for significance (p<0.05), n=3. A similar rate of cell growth in untreated samples in both model system was observed, however the efficacy of HSP 90 Inhibitor in reducing the tumor volume was no longer observed in the PDTs on the LLPI system.

### Example 7: Patient-Derived Tumoroid Platforms with Progressing Tumor Microenvironment Representability Reveals Potential Efficacy and Resistance Patterns in Pre-Clinical Regimen Testing for Improved Clinical Insights

Specifically, this study shows: I) customizing appropriately matched tumor-drug models for common cancers such as Triple Negative Breast Cancer (TNBC) and Gallbladder Cancer (GBC) against their established drug regimens in order to reveal potential efficacy and resistance insights across a justified platform spectrum of progressing TiME representability; II) investigating novel drug application for rare cancers via Human Uterine Adenosarcoma (HUAS) testing against Heat-Shock Protein 90-inhibitor HSP90i) regimens with the aid of broad TiME-feature spectrum COMPASS platforms in order to provide initial comparative insights for tumor-immune-drug interplays. Current findings highlighted the potential of matchingly-revealed efficacies (FIG. 28, I.A.2.iii, p=0.04) and potential conditions for optimal drug efficacy (FIG. 28, I.B.2.i, p=0.01; II, p=0.01-0.002). Overall, COMPASS' versatility and comprehensibility may aid in achieving efficient drug discovery.

Methods: COMPASS Platforms for TIME Modeling for Drug Regimen Testing: I.A. C1/C3a/C3c for TNBC-1 against 1) Doxorubicin 50 nM & 500 nM (Control: DMSO 0.01%), 2) Sacituzumab 5 nM & 20 nM [Control: PBS 0.01%] via continuous treatment w/initial administration at post-seeding Day 1; I.B. C2/C3b/C3d for GBC-1 against 1) Gemcitabine+Cisplatin 1 μM & 10 μM (G+C 7:1 ratio) (Control: DMSO 0.03%) 2) Gemcitabine+Cisplatin 1 μM+Durvalumab 0.1 nM; Gemcitabine+Cisplatin 10 μM+Durvalumab 1 nM (Control: DMSO 0.03%) (G+C 7:1 ratio) via continuous treatment w/initial administration at post-seeding Day 3; II. C1/C3b/C3d for HUAS-1 against 1) HSP90i 50 nM & 100 nM, (Control: DMSO 0.01-0.5%), 2) HSP90i 50 nM+Doxorubicin 50 nM & HSP90i 100 nM+Doxorubicin 50 nM, (Control: DMSO 0.01-0.5%) via continuous treatment w/initial administration at post-seeding Day 3;
Experimental Set-Up: Series (C1/C2) & representative parallel experimentation (C3), n=3 per group;
Evaluation: Imaging—Phase Contrast Imaging via Biotek Cytation 5 (Agilent) with partial well plug for object selection (20-200 μM for C1/C2 & 50-400 μM for C3) towards tumor volume analysis at Timepoints Day 3 and Day 7/10;
Statistics: One-way ANOVA followed by Tukey's pairwise comparison, n=3, (p<0.05). I.A.1: Doxorubicin-TNBC w/slight efficacy (C1) & potential resistance (C3); I.A.2: Sacituzumab-TNBC revealed efficacy in oxygenated conditions; I.B.1. G+C+Durvalumab efficacy showcased in simpler COMPASS models; II: HSP90i regimen efficacy showcased in simpler COMPASS models (consistent with HSP90i's pre-clinical vs clinical success discrepancy.

FIGS. 28A-L: Common Cancers (TNBC and GBC) vs Established Drug Regimens (Doxorubicin/Sacituzumab and Gemcitabine+Cisplatin+/−Durvalumab, respectively) treatment profiles across progressing, appropriately-matched COMPASS Platforms at Day 3 and Day 10 Timepoints. Compass 1 is used for small molecules. Compass 2 is used in studies when Durvalumab (which is an anti-PDL 1 inhibitor) is added and it works by using the immune system (checkpoint inhibitor), which makes the immune system stop/reduce activity. Sub-studies highlighting the versatility of COMPASS platforms in the generation of specific Tumor-Immune-Microenvironment Models—designed appropriately for intended drug mechanism testing of already clinically-approved treatment regimens.

Sub-study I.A. showcasing a range of non-immunocompetent platforms (C1, C3a, & C3c) for TME modeling of immunosuppressive TNBCs via the supplementation of additional structural matrix (C3—with TNBCs known to be well-formed and aggressive) and variable oxygenation (C3a/c—with TNBCs known to be hypoxic but with increased tendency of vascular invasion) in testing DNA-disrupting drugs (Doxorubicin & Sacituzumab). On the other hand, sub-study I.B. demonstrates the range of immunocompetent platforms (C2, C3b & C3d) for TiME modeling of GBCs via model feature supplementation with a similar rationale to that of TNBCs, but with further immunocompetence introduction to cater to Durvalumab's therapeutic mechanism (anti-PD-L1), aside from the DNA disrupting effects of Gemcitabine and Cisplatin. Whilst the tested doses of Doxorubicin showed varying potential efficacy and resistance trends (I.A.1), a separate Sacituzumab arm showcased tumor volume reduction vs control (blue arrows; p=0.04) in the presence of both supplemental structural matrix & oxygenation (I.A.2.iii). Sub-study I.B. showcased general resistance patterns for G+C only treatment-except for an early timepoint on a C3d model (I.B.1.iii Day 3); conversely, the baseline COMPASS 2 suggested potential G+C+Durvalumab efficacy via reduced tumoroid formation through time (red vs pink arrows) [p=0.01] compared to its corresponding feature-progressing models. n=3; p<0.05.

FIGS. 31A-C: Human Uterine Adenosarcoma (HUAS) vs HSP90i+/−Doxorubicin Treatment Profiles across progressing COMPASS Platforms at Day 3 & 7 Timepoints. Significant tumor volume reduction by HSP90i regimens in C1 model testing (**, p=0.002; *, p=0.01, via t-test) compared to C3 model testing. n=3; p<0.05.

### Example 8: Evaluate Various Modalities of Drug Treatment Using a Compass Platform for Pre-Clinical Studies

Additional studies will be performed to evaluate the efficacy of various modalities with small and large molecules, immunotherapeutic and combinations. Expected Outcome: Identify biomarkers associated with response to therapy using IHC, IF, RNA-seq, and proteomics.

Additional studies will be performed to evaluate the mechanism of action and tumor targeting properties of small molecules, large molecules, and immuno-therapeutics to optimize drug delivery.

### Example 9: Testing Functional Immunocompetence of the COMPASS Models in Different Immunotherapeutic Options

Additional studies will be performed to assess the impact of immune competence on tumor growth along with single cell RNA seq and spatial proteomic analysis to identify alterations of tumor microenvironment (TME).

Additional studies will be performed to test T-cell engagers, CAR-T and CAR-NK on the COMPASS-2 and COMPASS-3 model systems and characterize the impact of these therapeutics on tumor growth and TME.

Additional studies will be performed to identify and test neo-antigens like NYESO 1 in the Compass 2 and 3 model platform and test their impact on solid cancer immuno-therapeutics.

### Example 10: Testing the Impact of Physiological Oxygenation on Multiple Modalities of Treatment Using COMPASS 3

Additional studies will be performed to determine the impact of oxygenation status on the efficacy of clinically relevant small molecules, ADCs, CAR-T, and immune checkpoints using COMPASS 3 models.

Additional studies will be performed to investigate the genetic and proteomic changes observed upon oxygenation of TME in COMPASS models in context of therapeutics using single cell RNA seq and proteomics.

Additional studies will be performed to evaluate the impact of oxygenation induced pH alterations on the efficacy of cancer therapeutics.

The disclosure is understood by the following numbered paragraphs:
1. A construct of cells derived from a living object, comprising, consisting of, or consisting essentially of:
a plurality of cells derived from a living object;
at least one immune cell; and
a culturing matrix comprising, consisting of, or consisting essentially of exogenous laminin.
2. A construct of cells derived from a living object, comprising, consisting of, or consisting essentially of:
a plurality of cells derived from a living object; and
a culturing matrix comprising, consisting of, or consisting essentially of exogenous laminin.
wherein the construct of cells is supplied with oxygen from a media including oxygen.
3. A construct of cells derived from a living object, comprising, consisting of, or consisting essentially of:
a plurality of cells derived from a living object;
at least one immune cell; and
a culturing matrix comprising, consisting of, or consisting essentially of exogenous laminin, wherein the construct of cells is supplied with oxygen from a media including oxygen.
4. The construct of cells of claims 1 and 3, wherein the at least one immune cell constitutes about 1% to about 90% of the construct of cells.
5. The construct of cells of claims 1 and 3, wherein the at least one immune cell constitutes about 3% to about 80% of the construct of cells.
6. The construct of cells of claims 1 and 3, wherein the at least one immune cell constitutes about 5% to about 70% of the construct of cells.
7. The construct of cells of claims 1 and 3, wherein the at least one immune cell constitutes about 8% to about 65% of the construct of cells.
8. The construct of cells of claims 1 and 3, wherein the at least one immune cell constitutes about 10% to about 60% of the construct of cells.
9. The construct of cells of claims 1 and 3, wherein the at least one immune cell constitutes about 12% to about 55% of the construct of cells.
10. The construct of cells of claims 1 and 3, wherein the at least one immune cell constitutes about 15% to about 50% of the construct of cells.
11. The construct of cells of claims 1 and 3-10, wherein the at least one immune cell includes a mononuclear cell.
12. The construct of cells of claims 1 and 3-11, wherein the at least one immune cell includes a lymphocyte or a monocyte.
13. The construct of cells of claims 1 and 3-12, wherein the at least one immune cell comprises, consists of or consists essentially of a peripheral blood mononuclear cell (PBMC).
14. The construct of cells of claims 1 and 3-13, wherein the at least one immune cell comprises, consists of or consists essentially of T Cells (CD3+), B cells (CD20), Macrophages (CD68), NK cells (CD16), or a combination thereof.
15. The construct of cells of claims 1 and 3-14, wherein the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof.
16. The construct of cells of claim 15, wherein the information contains spatial information, temporal information, or a combination thereof.
17. The construct of cells of claim 15-16, wherein the information is based on at least two different indications from two different immune cells among the at least one immune cells.
18. The construct of cells of claim 15-17, wherein the at least one immune cell is tagged with a tagging molecule to indicate the information.
19. The construct of cells of claim 18, wherein the tagging molecule includes a fluorescent molecule, radioactive isotope molecule, coloring molecule, immunostaining molecule, or a combination thereof.
20. The construct of cells of claim 2-19, wherein the oxygen is supplied externally to the media via a gas-liquid interface.
21. The construct of cells of claim 20, wherein gas forming the gas-liquid interface has a lower oxygen concentration than ambient air.
22. The construct of cells of claim 20, wherein gas forming the gas-liquid interface has a higher oxygen concentration than ambient air.
23. The construct of cells of claim 20, wherein gas forming the gas-liquid interface includes from about 0% to about 90% of oxygen.
24. The construct of cells of claim 20, wherein gas forming the gas-liquid interface includes from about 0% to about 80% of oxygen.
25. The construct of cells of claim 20, wherein gas forming the gas-liquid interface includes

from about 0% to about 70% of oxygen.
26. The construct of cells of claim 20, wherein gas forming the gas-liquid interface includes from about 0% to about 60% of oxygen.
27. The construct of cells of claim 20, wherein gas forming the gas-liquid interface includes from about 0% to about 50% of oxygen.
28. The construct of cells of claim 20, wherein gas forming the gas-liquid interface includes from about 0% to about 40% of oxygen.
29. The construct of cells of claim 20, wherein gas forming the gas-liquid interface includes from about 0% to about 35% of oxygen.
30. The construct of cells of claim 20, wherein gas forming the gas-liquid interface includes from about 0% to about 30% of oxygen.
31. The construct of cells of claim 20, wherein gas forming the gas-liquid interface includes from about 0% to about 25% of oxygen.
32. The construct of cells of claim 20, wherein gas forming the gas-liquid interface includes from about 0% to about 22% of oxygen.
33. The construct of cells of claim 20, wherein gas forming the gas-liquid interface includes from about 0% to about 20% of oxygen.
34. The construct of cells of claim 2-33, wherein at least a portion of the media is replaced.
35. The construct of cells of claim 2-34, wherein at least a portion of the media is infused into a system containing the construct of cells for a first predetermined period of time.
36. The construct of cells of claim 35, wherein at least a portion of the media is withdrawn from the system for a second predetermined period of time.
37. The construct of cells of claim 2-36, wherein the media includes a blood substitute.
38. The construct of cells of claim 2-36, wherein the media includes hemoglobin.
39. The construct of cells of claim 2-36, wherein the media includes synthetic hemoglobin.
40. The construct of cells of claim 2-39, wherein the media comprises a first media and a second media different from the first media.
41. The construct of cells of claim 40, wherein the second media includes blood substitute.
42. The construct of cells of claim 40, wherein the second media includes hemoglobin.
43. The construct of cells of claim 40, wherein the second media includes artificial hemoglobin.
44. The construct of cells of claim 40-43, wherein the density of the first media is lower than the second media.
45. The construct of cells of claim 40-44, wherein the first media is in contact with cells of the plurality of cells.
46. The construct of cells of claim 40-45, wherein the second media forms a liquid-liquid interface with the first media.
47. The construct of cells of claim 40-46, wherein the concentration of oxygen in the second media is from about 0 μM to about 300 μM.
48. The construct of cells of claim 40-46, wherein the concentration of oxygen in the second media is from about 0 μM to about 270 μM.
49. The construct of cells of claim 40-46, wherein the concentration of oxygen in the second media is from about 0 μM to about 250 μM.
50. The construct of cells of claim 40-46, wherein the concentration of oxygen in the second media is from about 0 μM to about 240 μM.
51. The construct of cells of claim 40-46, wherein the concentration of oxygen in the second media is from about 0 μM to about 235 μM.
52. The construct of cells of claim 40-46, wherein the concentration of oxygen in the second media is from about 0 μM to about 230 μM.
53. The construct of cells of claim 40-46, wherein the concentration of oxygen in the second media is from about 0 μM to about 225 μM.
54. The construct of cells of claim 40-46, wherein the concentration of oxygen in the second media is from about 0 μM to about 200 μM.
55. The construct of cells of claim 40-46, wherein the concentration of oxygen in the second media is from about 0 M to about 175 μM.
56. The construct of cells of claim 40-46, wherein the concentration of oxygen in the second media is from about 0 μM to about 150 μM.
57. The construct of cells of claim 40-46, wherein the concentration of oxygen in the second media is from about 0 μM to about 125 μM.
58. The construct of cells of claim 40-46, wherein the concentration of oxygen in the second media is from about 0 μM to about 100 μM.
59. The construct of cells of claim 40-46, wherein the concentration of oxygen in the second media is from about 0 μM to about 75 μM.
60. The construct of cells of claim 40-46, wherein the concentration of oxygen in the second media is from about 0 μM to about 50 μM.
61. The construct of cells of claim 40-46, wherein the concentration of oxygen in the second media is from about 0 M to about 25 μM.
62. The construct of cells of claim 40-46, wherein the concentration of oxygen in the second media is from about 25 μM to about 300 μM.
63. The construct of cells of claim 40-46, wherein the concentration of oxygen in the second media is from about 25 μM to about 270 μM.
64. The construct of cells of claim 40-46, wherein the concentration of oxygen in the second media is from about 25 μM to about 250 μM.
65. The construct of cells of claim 40-46, wherein the concentration of oxygen in the second media is from about 25 μM to about 240 μM.
66. The construct of cells of claim 40-46, wherein the concentration of oxygen in the second media is from about 25 μM to about 235 μM.
67. The construct of cells of claim 40-46, wherein the concentration of oxygen in the second media is from about 25 μM to about 230 μM.
68. The construct of cells of claim 40-46, wherein the concentration of oxygen in the second media is from about 25 μM to about 225 μM.
69. The construct of cells of claim 40-46, wherein the concentration of oxygen in the second media is from about 25 μM to about 200 μM.
70. The construct of cells of claim 40-46, wherein the concentration of oxygen in the second media is from about 25 μM to about 175 μM.
71. The construct of cells of claim 40-46, wherein the concentration of oxygen in the second media is from about 25 μM to about 150 μM.
72. The construct of cells of claim 40-46, wherein the concentration of oxygen in the second media is from about 25 μM to about 125 μM.
73. The construct of cells of claim 40-46, wherein the concentration of oxygen in the second media is from about 25 μM to about 100 μM.
74. The construct of cells of claim 40-46, wherein the concentration of oxygen in the second media is from about 25 μM to about 75 μM.
75. The construct of cells of claim 40-46, wherein the concentration of oxygen in the second media is from about 25 μM to about 50 μM.
76. The construct of cells of claim 1-75, wherein the culturing matrix comprises exogenous entactin.
77. The construct of cells of claim 1-76, wherein the culturing matrix comprises Matrigel™.
78. The construct of cells of claim 1-77, wherein the culturing matrix induces more cell proliferation of the construct of cells within a given period of time.
79. The construct of cells of claim 1-78, wherein the culturing matrix induces more mass of the construct of cells within a given period of time.
80. The construct of cells of claim 1-79, wherein the culturing matrix induces more volume of the construct of cells within a given period of time.
81. The construct of cells of claim 1-80, wherein the construct of cells includes a three-dimensional (3D) structure.
82. The construct of cells of claim 1-81, wherein the construct of cells includes a three-dimensional (3D) patient-derived tumoroid (PDTs).
83. The construct of cells of claim 1-82, wherein the plurality of cells is derived from a tumor of the living object.
84. The construct of cells of claim 1-83, wherein the plurality of cells includes cancer cells.
85. The construct of cells of claim 1-84, wherein the plurality of cells includes sarcoma cells, gallbladder cancer,

86. The construct of cells of claim 1-85, wherein the plurality of cells includes Human Uterine Adenosarcoma cells.
87. A system for a construct of cells, comprising, consisting of, or consisting essentially of:
a tissue container to grow a construct of cells constructed in the tissue container, wherein the construct comprises:
a plurality of cells derived from a living object;
at least one immune cell; and
a culturing matrix comprising, consisting of, or consisting essentially of exogenous laminin.
88. a system to contain a construct of cells, comprising, consisting of, or consisting essentially of:
a tissue container to grow a construct of cells constructed in the tissue container, wherein the construct comprises:
a plurality of cells derived from a living object; and
a culturing matrix comprising, consisting of, or consisting essentially of exogenous laminin,
wherein the construct of cells is supplied with oxygen from a media including oxygen.
89. A system for a construct of cells, comprising, consisting of, or consisting essentially of:
a tissue container to grow a construct of cells constructed in the tissue container, wherein the construct comprises:
a plurality of cells derived from a living object;
at least one immune cell; and
a culturing matrix comprising, consisting of, or consisting essentially of exogenous laminin,
wherein the construct of cells is to be supplied with oxygen from a media including oxygen.
90. The system for the construct of cells of claims 87 and 89, wherein the at least one immune cell constitutes about 1% to about 90% of the construct of cells.
91. The system for the construct of cells of claims 87 and 89, wherein the at least one immune cell constitutes about 3% to about 80% of the construct of cells.
92. The system for the construct of cells of claims 87 and 89, wherein the at least one immune cell constitutes about 5% to about 70% of the construct of cells.
93. The system for the construct of cells of claims 87 and 89, wherein the at least one immune cell constitutes about 8% to about 65% of the construct of cells.
94. The system for the construct of cells of claims 87 and 89, wherein the at least one immune cell constitutes about 10% to about 60% of the construct of cells.
95. The system for the construct of cells of claims 87 and 89, wherein the at least one immune cell constitutes about 12% to about 55% of the construct of cells.
96. The system for the construct of cells of claims 87 and 89, wherein the at least one immune cell constitutes about 15% to about 50% of the construct of cells.
97. The system for the construct of cells of claims 87 and 89-96, wherein the at least one immune cell includes a mononuclear cell.
98. The system for the construct of cells of claims 87 and 89-97, wherein the at least one immune cell includes a lymphocyte or a monocyte.
99. The system for the construct of cells of claims 87 and 89-98, wherein the at least one immune cell includes a peripheral blood mononuclear cell (PBMC).
100. The system for the construct of cells of claims 87 and 89-99, wherein the at least one immune cell includes T Cells (CD3+), B cells (CD20), Macrophages (CD68), NK cells (CD16), or a combination thereof.
101. The system for the construct of cells of claims 87 and 89-100, wherein the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof.
102. The system for the construct of cells of claim 101, wherein the information contains spatial information, temporal information, or a combination thereof.
103. The system for the construct of cells of claim 101-102, wherein the information is based on at least two different indications from two different immune cells among the at least one immune cells.
104. The system for the construct of cells of claim 101-103, wherein the at least one immune cell is tagged with a tagging molecule to indicate the information.
105. The system for the construct of cells of claim 104, wherein the tagging molecule includes a fluorescent molecule, radioactive isotope molecule, coloring molecule, immunostaining molecule, or a combination thereof.
106. The system for the construct of cells of claim 88-103, wherein the oxygen is perfused externally to the media via a gas-liquid interface.
107. The system for the construct of cells of claim 106, wherein gas forming the gas-liquid interface has lower oxygen concentration than ambient air.
108. The system for the construct of cells of claim 106, wherein gas forming the gas-liquid interface has higher oxygen concentration than ambient air.
109. The system for the construct of cells of claim 106, wherein gas forming the gas-liquid interface includes from about 0% to about 90% of oxygen.
110. The system for the construct of cells of claim 106, wherein gas forming the gas-liquid interface includes from about 0% to about 80% of oxygen.
111. The system for the construct of cells of claim 106, wherein gas forming the gas-liquid interface includes from about 0% to about 70% of oxygen.
112. The system for the construct of cells of claim 106, wherein gas forming the gas-liquid interface includes from about 0% to about 60% of oxygen.
113. The system for the construct of cells of claim 106, wherein gas forming the gas-liquid interface includes from about 0% to about 50% of oxygen.
114. The system for the construct of cells of claim 106, wherein gas forming the gas-liquid interface includes from about 0% to about 40% of oxygen.
115. The system for the construct of cells of claim 106, wherein gas forming the gas-liquid interface includes from about 0% to about 35% of oxygen.
116. The system for the construct of cells of claim 106, wherein gas forming the gas-liquid interface includes from about 0% to about 30% of oxygen.
117. The system for the construct of cells of claim 106, wherein gas forming the gas-liquid interface includes from about 0% to about 25% of oxygen.
118. The system for the construct of cells of claim 106, wherein gas forming the gas-liquid interface includes from about 0% to about 22% of oxygen.
119. The system for the construct of cells of claim 106, wherein gas forming the gas-liquid interface includes from about 0% to about 20% of oxygen.
120. The system for the construct of cells of claim 88-119, wherein at least a portion of the media in the system is replaced.
121. The system for the construct of cells of claim 88-120, wherein at least a portion of the media is infused into a system containing the construct of cells for a first predetermined period of time.
122. The system for the construct of cells of claim 121, wherein at least a portion of the media is withdrawn from the system for a second predetermined period of time.
123. The system for the construct of cells of claim 88-122, wherein the media includes a blood substitute.
124. The system for the construct of cells of claim 88-122, wherein the media includes hemoglobin.
125. The system for the construct of cells of claim 88-122, wherein the media includes synthetic hemoglobin.
126. The system for the construct of cells of claim 88-125, wherein the media comprises a first media and a second media different from the first media.
127. The system for the construct of cells of claim 126, wherein second media includes blood substitute.
128. The system for the construct of cells of claim 126, wherein second media includes hemoglobin.
129. The system for the construct of cells of claim 126, wherein second media includes artificial hemoglobin.
130. The system for the construct of cells of claim 126-129, wherein the density of the first media is lower than the second media.
131. The construct of cells of claim 126-130, wherein the first media is in contact with cells of the plurality of cells.
132. The construct of cells of claim 126-131, wherein the second media forms a liquid-liquid interface with the first media.
133. The system for the construct of cells of claim 126-132, wherein the concentration of oxygen in the second media is from about 0 μM to about 300 μM.
134. The system for the construct of cells of claim 126-132, wherein the concentration of oxygen in the second media is from about 0 μM to about 270 μM.
135. The system for the construct of cells of claim 126-132, wherein the concentration of oxygen in the second media is from about 0 μM to about 250 μM.
136. The system for the construct of cells of claim 126-132, wherein the concentration of oxygen in the second media is from about 0 μM to about 240 μM.
137. The system for the construct of cells of claim 126-132, wherein the concentration of oxygen in the second media is from about 0 μM to about 235 μM.
138. The system for the construct of cells of claim 126-132, wherein the concentration of oxygen in the second media is from about 0 μM to about 230 μM.
139. The system for the construct of cells of claim 126-132, wherein the concentration of oxygen in the second media is from about 0 μM to about 225 μM.
140. The system for the construct of cells of claim 126-132, wherein the concentration of oxygen in the second media is from about 0 μM to about 200 μM.
141. The system for the construct of cells of claim 126-132, wherein the concentration of oxygen in the second media is from about 0 μM to about 175 μM.
142. The system for the construct of cells of claim 126-132, wherein the concentration of oxygen in the second media is from about 0 μM to about 150 μM.
143. The system for the construct of cells of claim 126-132, wherein the concentration of oxygen in the second media is from about 0 μM to about 125 μM.
144. The system for the construct of cells of claim 126-132, wherein the concentration of oxygen in the second media is from about 0 μM to about 100 μM.
145. The system for the construct of cells of claim 126-132, wherein the concentration of oxygen in the second media is from about 0 μM to about 75 μM.
146. The system for the construct of cells of claim 126-132, wherein the concentration of oxygen in the second media is from about 0 μM to about 50 μM.
147. The system for the construct of cells of claim 126-132, wherein the concentration of oxygen in the second media is from about 0 μM to about 25 μM.
148. The system for the construct of cells of claim 126-132, wherein the concentration of oxygen in the second media is from about 25 μM to about 300 μM.
149. The system for the construct of cells of claim 126-132, wherein the concentration of oxygen in the second media is from about 25 μM to about 270 μM.
150. The system for the construct of cells of claim 126-132, wherein the concentration of oxygen in the second media is from about 25 μM to about 250 μM.
151. The system for the construct of cells of claim 126-132, wherein the concentration of oxygen in the second media is from about 25 μM to about 240 μM.
152. The system for the construct of cells of claim 126-132, wherein the concentration of oxygen in the second media is from about 25 μM to about 235 μM.
153. The system for the construct of cells of claim 126-132, wherein the concentration of oxygen in the second media is from about 25 μM to about 230 μM.
154. The system for the construct of cells of claim 126-132, wherein the concentration of oxygen in the second media is from about 25 μM to about 225 μM.
155. The system for the construct of cells of claim 126-132, wherein the concentration of oxygen in the second media is from about 25 μM to about 200 μM.
156. The system for the construct of cells of claim 126-132, wherein the concentration of oxygen in the second media is from about 25 μM to about 175 μM.
157. The system for the construct of cells of claim 126-132, wherein the concentration of oxygen in the second media is from about 25 μM to about 150 μM.
158. The system for the construct of cells of claim 126-132, wherein the concentration of oxygen in the second media is from about 25 μM to about 125 μM.
159. The system for the construct of cells of claim 126-132, wherein the concentration of oxygen in the second media is from about 25 μM to about 100 μM.
160. The system for the construct of cells of claim 126-132, wherein the concentration of oxygen in the second media is from about 25 μM to about 75 μM.
161. The system for the construct of cells of claim 126-132, wherein the concentration of oxygen in the second media is from about 25 μM to about 50 μM.
162. The system for the construct of cells of claim 87-161, wherein the culturing matrix further comprises exogenous entactin.
163. The system for the construct of cells of claim 87-162, wherein the culturing matrix comprises Matrigel™.

164. The system for the construct of cells of claim 87-163, wherein the culturing matrix is to induce more cell proliferation of the construct of cells within a given period of time.
165. The system for the construct of cells of claim 87-164, wherein the culturing matrix is to induce more mass of the construct of cells within a given period of time.
166. The system for the construct of cells of claim 87-165, wherein the culturing matrix is to induce more volume of the construct of cells within a given period of time.
167. The system for the construct of cells of claim 87-166, wherein the construct of cells includes a three-dimensional (3D) structure.
168. The system for the construct of cells of claim 87-167, wherein the construct of cells includes a three-dimensional (3D) patient-derived tumoroid (PDTs).
169. The system for the construct of cells of claim 87-168, wherein the plurality of cells is derived from a tumor of the living object.
170. The system for the construct of cells of claim 87-169, wherein the plurality of cells includes cancer cells.
171. The system for the construct of cells of claim 87-170, wherein the plurality of cells includes sarcoma cells, gallbladder cancer,
172. The system for the construct of cells of claim 87-171, wherein the plurality of cells includes Human Uterine Adenosarcoma cells.
173. The system for the construct of cells of claim 172, further comprising, consisting of, or consisting essentially of an inlet to allow a medium carrying oxygen to enter the system to perfuse oxygen to be supplied to the construct of cells.
174. The system for the construct of cells of claim 87-173, further comprising, consisting of, or consisting essentially of a well to contain the construct of the cells.
175. A method of evaluating a response of a plurality of cells derived from a living object against an external stimulus, the method comprising, consisting of, or consisting essentially of:
culturing a construct of cells comprising, consisting of, or consisting essentially of a plurality of cells derived from a living object and at least one immune cell, in a culturing matrix comprising, consisting of, or consisting essentially of exogenous laminin; and subjecting the plurality of cultured cells to an external stimulus.
176. A method of evaluating a response of a plurality of cells derived from a living object against an external stimulus, the method comprising, consisting of, or consisting essentially of:
culturing a construct of cells comprising, consisting of, or consisting essentially of a plurality of cells derived from a living object in a culturing matrix comprising, consisting of, or consisting essentially of exogenous laminin; and subjecting the plurality of cultured cells to an external stimulus.
wherein the plurality of cells is supplied with oxygen from a media including oxygen.
177. A method of evaluating a response of a plurality of cells derived from a living object against an external stimulus, the method comprising, consisting of, or consisting essentially of:
culturing a construct of cells comprising, consisting of, or consisting essentially of a plurality of cells derived from a living object and at least one immune cell, in a culturing matrix comprising, consisting of, or consisting essentially of exogenous laminin; and subjecting the plurality of cultured cells to an external stimulus,
wherein the plurality of cells is supplied with oxygen from a media including oxygen.
178. The method of claims 175 and 177, wherein the at least one immune cell constitutes about 1% to about 90% of the construct of cells.
179. The method of claims 175 and 177, wherein the at least one immune cell constitutes about 3% to about 80% of the construct of cells.
180. The method of claims 175 and 177, wherein the at least one immune cell constitutes about 5% to about 70% of the construct of cells.
181. The method of claims 175 and 177, wherein the at least one immune cell constitutes about 8% to about 65% of the construct of cells.
182. The method of claims 175 and 177, wherein the at least one immune cell constitutes about 10% to about 60% of the construct of cells.
183. The method of claims 175 and 177, wherein the at least one immune cell constitutes about 12% to about 55% of the construct of cells.
184. The method of claims 175 and 177, wherein the at least one immune cell constitutes about 15% to about 50% of the construct of cells.
185. The method of claims 175 and 177-184, wherein the at least one immune cell includes a mononuclear cell.
186. The method of claims 175 and 177-185, wherein the at least one immune cell includes a lymphocyte or a monocyte.
187. The method of claims 175 and 177-186, wherein the at least one immune cell includes a peripheral blood mononuclear cell (PBMC).
188. The method of claims 175 and 177-187, wherein the at least one immune cell includes T Cells (CD3+), B cells (CD20), Macrophages (CD68), NK cells (CD16), or a combination thereof.
189. The method of claims 175 and 177-188, wherein the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof.
190. The method of claim 189, wherein the information contains spatial information, temporal information, or a combination thereof.
191. The method of claim 189-190, wherein the information is based on at least two different indications from two different immune cells among the at least one immune cells.
192. The method of claim 189-191, wherein the at least one immune cell is tagged with a tagging molecule to indicate the information.
193. The method of claim 192, wherein the tagging molecule includes a fluorescent molecule, radioactive isotope molecule, coloring molecule, immunostaining molecule, or a combination thereof.
194. The method of claim 176-193, wherein the oxygen is supplied externally to the media via a gas-liquid interface.
195. The method of claim 194, wherein gas forming the gas-liquid interface has lower oxygen concentration than ambient air.
196. The method of claim 194, wherein gas forming the gas-liquid interface has higher oxygen concentration than ambient air.
197. The method of claim 194, wherein gas forming the gas-liquid interface includes from about 0% to about 90% of oxygen.
198. The method of claim 194, wherein gas forming the gas-liquid interface includes from about 0% to about 80% of oxygen.
199. The method of claim 194, wherein gas forming the gas-liquid interface includes from about 0% to about 70% of oxygen.
200. The method of claim 194, wherein gas forming the gas-liquid interface includes from about 0% to about 60% of oxygen.
201. The method of claim 194, wherein gas forming the gas-liquid interface includes from about 0% to about 50% of oxygen.
202. The method of claim 194, wherein gas forming the gas-liquid interface includes from about 0% to about 40% of oxygen.
203. The method of claim 194, wherein gas forming the gas-liquid interface includes from about 0% to about 35% of oxygen.
204. The method of claim 194, wherein gas forming the gas-liquid interface includes from about 0% to about 30% of oxygen.
205. The method of claim 194, wherein gas forming the gas-liquid interface includes from about 0% to about 25% of oxygen.
206. The method of claim 194, wherein gas forming the gas-liquid interface includes from about 0% to about 22% of oxygen.
207. The method of claim 194, wherein gas forming the gas-liquid interface includes from about 0% to about 20% of oxygen.
208. The method of claim 176-207, wherein at least a portion of the media is replaced.
209. The method of claim 176-208, wherein at least a portion of the media is infused into a system containing the construct of cells for a first predetermined period of time.
210. The method of claim 209, wherein at least a portion of the media is withdrawn from the system for a second predetermined period of time.
211. The method of claim 176-210, wherein the media includes a blood substitute.
212. The method of claim 176-210, wherein the media includes hemoglobin.
213. The method of claim 176-210, wherein the media includes synthetic hemoglobin.
214. The method of claim 176-213, wherein the media comprises a first media and a second media different from the first media.
215. The method of claim 214, wherein the second media includes blood substitute.
216. The method of claim 214, wherein the second media includes hemoglobin.
217. The method of claim 214, wherein the second media includes artificial hemoglobin.
218. The method of claim 214-217, wherein the density of the first media is lower than the second media.
219. The method of claim 214-218, wherein the first media is in contact with cells of the plurality of cells.
220. The method of claim 214-219, wherein the second media forms a liquid-liquid interface with the first media.
221. The method of claim 214-220, wherein the concentration of oxygen in the second media is from about 0 μM to about 300 μM.
222. The method of claim 214-220, wherein the concentration of oxygen in the second media is from about 0 μM to about 270 μM.
223. The method of claim 214-220, wherein the concentration of oxygen in the second media is from about 0 μM to about 250 μM.
224. The method of claim 214-220, wherein the concentration of oxygen in the second media is from about 0 μM to about 240 μM.
225. The method of claim 214-220, wherein the concentration of oxygen in the second media is from about 0 μM to about 235 μM.
226. The method of claim 214-220, wherein the concentration of oxygen in the second media is from about 0 M to about 230 μM.
227. The method of claim 214-220, wherein the concentration of oxygen in the second media is from about 0 μM to about 225 μM.
228. The method of claim 214-220, wherein the concentration of oxygen in the second media is from about 0 μM to about 200 μM.
229. The method of claim 214-220, wherein the concentration of oxygen in the second media is from about 0 μM to about 175 μM.
230. The method of claim 214-220, wherein the concentration of oxygen in the second media is from about 0 μM to about 150 μM.
231. The method of claim 214-220, wherein the concentration of oxygen in the second media is from about 0 μM to about 125 μM.
232. The method of claim 214-220, wherein the concentration of oxygen in the second media is from about 0 μM to about 100 μM.
233. The method of claim 214-220, wherein the concentration of oxygen in the second media is from about 0 μM to about 75 μM.
234. The method of claim 214-220, wherein the concentration of oxygen in the second media is from about 0 μM to about 50 μM.
235. The method of claim 214-220, wherein the concentration of oxygen in the second media is from about 0 μM to about 25 μM.
236. The method of claim 214-220, wherein the concentration of oxygen in the second media is from about 25 μM to about 300 μM.
237. The method of claim 214-220, wherein the concentration of oxygen in the second media is from about 25 μM to about 270 μM.
238. The method of claim 214-220, wherein the concentration of oxygen in the second media is from about 25 μM to about 250 μM.
239. The method of claim 214-220, wherein the concentration of oxygen in the second media is from about 25 μM to about 240 μM.
240. The method of claim 214-220, wherein the concentration of oxygen in the second media is from about 25 μM to about 235 μM.
241. The method of claim 214-220, wherein the concentration of oxygen in the second media is from about 25 μM to about 230 μM.
242. The method of claim 214-220, wherein the concentration of oxygen in the second media is from about 25 μM to about 225 μM.
243. The method of claim 214-220, wherein the concentration of oxygen in the second media is from about 25 μM to about 200 μM.
244. The method of claim 214-220, wherein the concentration of oxygen in the second media is from about 25 μM to about 175 μM.
245. The method of claim 214-220, wherein the concentration of oxygen in the second media is from about 25 μM to about 150 μM.
246. The method of claim 214-220, wherein the concentration of oxygen in the second media is from about 25 μM to about 125 μM.
247. The method of claim 214-220, wherein the concentration of oxygen in the second media is from about 25 μM to about 100 μM.
248. The method of claim 214-220, wherein the concentration of oxygen in the second media is from about 25 μM to about 75 μM.
249. The method of claim 214-220, wherein the concentration of oxygen in the second media is from about 25 μM to about 50 μM.
250. The method of claim 175-249, wherein the external stimulus is based on a small molecule therapeutics.
251. The method of claim 175-250, wherein the external stimulus is based on a large molecule therapeutics.
252. The method of claim 175-251, wherein the culturing matrix further comprises exogenous entactin.
253. The method of claim 175-252, wherein the culturing matrix comprises Matrigel™.
254. The method of claim 175-253, wherein the culturing matrix induces more cell proliferation of the construct of cells within a given period of time.
255. The method of claim 175-253, wherein the culturing matrix induces more mass of the construct of cells within a given period of time.
256. The method of claim 175-253, wherein the culturing matrix induces more volume of the construct of cells within a given period of time.
257. The method of claim 175-256, wherein the construct of cells includes a three-dimensional (3D) structure.
258. The method of claim 175-257, wherein the construct of cells includes a three-dimensional (3D) patient-derived tumoroid (PDTs).
259. The method of claim 175-258, wherein the plurality of cells is derived from a tumor of the living object.
260. The method of claim 175-259, wherein the plurality of cells includes cancer cells.
261. The method of claim 175-260, wherein the plurality of cells includes sarcoma cells, gallbladder cancer,
262. The method of claim 175-261, wherein the plurality of cells includes Human Uterine Adenosarcoma cells.
263. The method or construct of cells of any preceding claim wherein the PDT is functionally immunocompetent and has physiological oxygenation.

What is claimed is:

1. A method of evaluating a response of a tumoroid of cells derived from a human patient against an external stimulus, the method comprising, consisting of, or consisting essentially of:
culturing a construct of cells comprising, consisting of, or consisting essentially of a plurality of cells derived from the human patient and at least one immune cell, in a culturing matrix comprising, consisting of, or consisting essentially of exogenous laminin and wherein the construct of cells is supplied with oxygen from a media including oxygen; and
subjecting the plurality of cultured cells to an external stimulus.

2. The method of claim 1, wherein the construct of cells is functionally immunocompetent and has physiological oxygenation.

3. The method of claim 1, further comprising forming a liquid-liquid interface below, above or bisecting the construct of cells by a first media and a second media wherein the first media and second media are different, and the first media has a lower density and the second media comprises oxygen from about 1 μM to about 300 μM.

4. The method of claim 1, wherein the at least one immune cell constitutes about 1% to about 90% of the construct of cells.

5. The method of claim 1, wherein the at least one immune cell includes a mononuclear cell, a lymphocyte, a monocyte, a peripheral blood mononuclear cell (PBMC), T Cells (CD3+), B cells (CD20), Macrophages (CD68), NK cells (CD16), or a combination thereof.

6. The method of claim 1, wherein the at least one immune cell in the construct of cells is distributed or located in the construct to indicate information related to a tumoroid infiltration of an immune component, an immunophenotype, an immunosuppressive mechanism, an immunomodulatory mechanisms, or a combination thereof.

7. The method of claim 6, wherein the information contains spatial information, temporal information, or a combination thereof.

8. The method of claim 6, wherein the information is based on at least two different indications from two different immune cells among the at least one immune cells.

9. The method of claim 6, wherein the at least one immune cell is tagged with a tagging molecule to indicate the information, wherein the tagging molecule includes a fluorescent molecule, radioactive isotope molecule, coloring molecule, immunostaining molecule, or a combination thereof.

10. The method of claim 1, wherein the PDT is functionally immunocompetent or has physiological oxygenation.

11. The method of claim 10, wherein gas forming the gas-liquid interface has lower oxygen concentration than ambient air or the gas-liquid interface has higher oxygen concentration than ambient air.

12. The method of claim 10, wherein gas forming the gas-liquid interface includes from about 0% to about 90% of oxygen.

13. The method of claim 1, wherein at least a portion of the media is infused/added into a system containing the construct of cells for a first predetermined period of time.

14. The method of claim 13, wherein at least a portion of the media is withdrawn from the system for a second predetermined period of time wherein the first period of time and second period of time are different.

15. The method of claim 1, wherein the media includes a blood substitute.

16. The method of claim 15, wherein the media comprises a first media and a second media different from the first media.

17. The method of claim 16, wherein the density of the first media is lower than the second media.

18. The method of claim 16, wherein the second media forms a liquid-liquid interface with the first media.

19. The method of claim 16, wherein the concentration of oxygen in the second media is from about 0 μM to about 300 μM.

20. The method of claim 1, wherein the external stimulus is based on a small molecule therapeutics or large molecule therapeutics.
